(19) United States
(12) Patent Application Publication
(10) Pub. No.: US 2025/0064871 A1
(43) Pub. Date: Feb. 27, 2025

(54) BACTERIAL COMPOSITIONS AND METHODS FOR TREATING CANCER AND IMMUNE DISEASES

(71) Applicant: The Regents of The University of California, Oakland, CA (US)

(72) Inventor: Anahid Jewett, Los Angeles, CA (US)

(21) Appl. No.: 18/728,163

(22) PCT Filed: Jan. 18, 2023
(86) PCT No.: PCT/US23/10989

§ 371 Date: Jul. 11, 2024

Related U.S. Application Data
(60) Provisional application No. 63/433,118, filed on Dec. 16, 2022.
(60) Provisional application No. 63/300,368, filed on Jan. 18, 2022.

(51) Int. Cl. A61K 35/745; A61K 31/198; A61K 31/337; A61K 31/4985; A61K 31/519; A61K 33/243; A61K 35/00; A61K 35/744; A61K 35/747; A61K 39/00; A61K 39/395; A61P 35/00

(52) U.S. Cl. CPC A61K 35/745; A61K 31/198; A61K 31/337; A61K 31/4985; A61K 31/519; A61K 33/243; A61K 35/744; A61K 35/747; A61K 39/3955; A61K 39/4613; A61P 35/00; A61K 2035/115

(57) ABSTRACT

The present application relates to probiotic compositions comprising two or more bacterial strains selected from: Bifidobacterium longum, Bifidobacterium breve, Bifidobacterium infantis, Streptococcus thermophiles, Lactobacillus acidophilus, Lactobacillus plantarum, and Lactobacillus paracasei.

### CROSS-REFERENCE TO RELATED APPLICATIONS

This application claims the benefit of U.S. Provisional Application No. 63/300,368, filed Jan. 18, 2022, and U.S. Provisional Application No. 63/433,118, filed Dec. 16, 2022. The entire contents of each of said applications are incorporated herein in their entirety by this reference.

### BACKGROUND

Natural killer (NK) cells are immune cells that develop in the bone marrow and constitute about 5-10% of lymphocytes in the peripheral blood and secondary lymphoid organs. NK cell effector functions include direct cytotoxicity, antibody-dependent cellular cytotoxicity (ADCC), and inflammatory cytokines and chemokines secretion. These secreted factors regulate other immune cells' functions.

While cytokines and chemokines produced by NK cells are important in modulating the immune response in diseased (e.g., those afflicted with an inflammatory/autoimmune disease or cancer) and healthy individuals, a therapy that modulates these cytokines and chemokines has been challenging due to a lack of suitable agents and methods. Accordingly, there is a great need in the art for compositions and methods for modulating the level of cytokines and chemokines that can be used to prevent or treat diseases (e.g., inflammatory/autoimmune diseases or cancer) as well as those for maintaining optimal immune response in healthy individuals.

### SUMMARY OF THE INVENTION

The present invention is based, at least in part, on the discovery that certain combinations of probiotic bacteria differentially regulate production or secretion of cytokines and chemokines. Notably, bacteria of Bifidobacterium species preferentially induce NK cells to secrete anti-inflammatory cytokines (e.g., G-CSF, Gro-alpha, IL-10) that are useful in preventing or treating inflammatory diseases or autoimmune diseases. By contrast, bacteria of Lactobacillus species preferentially induce NK cells to secrete proinflammatory cytokines (e.g., IFN-γ) that are useful in preventing or treating cancer. In addition, a combination of both bacteria species is useful in balancing the level of proinflammatory and anti-inflammatory cytokines and chemokines in healthy and diseased individuals.

### BRIEF DESCRIPTION OF THE DRAWINGS

FIG. 1 shows higher secretion of IFN-g by AJ4 treatment in comparison to AJ3 treatment in NK cells, when NK cells at (1×10⁶ cells/ml) were treated with IL-2 (1000 u/ml) and each of AJ3 and AJ4 at (20:1 NK to bacteria) treatment.

FIG. 2 shows higher secretion of IFN-g by AJ4 treatment in comparison to AJ3 treatment in NK cells, when NK cells at (1×10⁶ cells/ml) were treated with IL-2 (1000 u/ml) and each of AJ3 and AJ4 at (20:1 NK to bacteria) treatment.

FIG. 3A-FIG. 3G show increased IFN-γ and decreased IL-10 secretions by sAJ4 treated PBMCs in comparison to sAJ3 and sAJ2 treated PBMCs. PBMCs were left untreated or treated with IL-2 (1000 U/ml) or with a combination of IL-2 (1000 U/ml) and anti-CD16 mAbs (3 μg/ml) or with sAJ3 (PBMC: sAJ3, 1:20) or with sAJ4 (PBMC: sAJ4, 1:20) or with a combination of IL-2 (1000 U/ml) and sAJ3 (PBMC: sAJ3, 1:20) or with a combination of IL-2 (1000 U/ml) and sAJ4 (PBMC: sAJ4, 1:20) for 18 hours before the supernatants were harvested from PBMCs to determine IFN-γ (FIG. 3A), TNF-α (FIG. 3B), IL-6 (FIG. 3C), and IL-10 (FIG. 3D) secretion using multiplex assay. PBMCs were left untreated or treated with IL-2 (1000 U/ml) or with a combination of IL-2 (1000 U/ml) and anti-CD16 mAbs (3 μg/ml) or with a combination of IL-2 (1000 U/ml) and anti-CD3/28 antibody (25 μl/ml) or with a combination of IL-2 (1000 U/ml) and sAJ2 (PBMC: sAJ2, 1:20) or with a combination of IL-2 (1000 U/ml) and sAJ3 (PBMC: sAJ3, 1:20) or with a combination of IL-2 (1000 U/ml) and sAJ4 (PBMC: sAJ4, 1:20) or with sAJ2 (PBMC: sAJ2, 1:20) or with sAJ3 (PBMC: sAJ3, 1:20) or with sAJ4 (PBMC: sAJ4, 1:20) for 18 hours before the supernatants were harvested from PBMCs to determine IFN-γ secretion using single ELISA. One of the representative experiments is shown (FIG. 3E), and cumulative samples are shown (n=5) (FIG. 3F). PBMCs were treated as described in FIG. 3E for 18 hours before the supernatants were harvested from PBMCs to determine IFN-γ and IL-10 secretion using specific single ELISAs, and ratio of IFN-γ to IL-10 was determined (FIG. 3G). **(p value 0.001-0.01), *(p value 0.01-0.05).

FIG. 4A-FIG. 4I show increased IFN-γ and decreased IL-10 secretions by sAJ4 treated NK cells in comparison to sAJ3 and sAJ2 treated NK cells. NK cells were left untreated or treated with IL-2 (1000 U/ml) or with a combination of IL-2 (1000 U/ml) and anti-CD16 mAbs (3 μg/ml) or with sAJ3 (NK: sAJ3, 1:20) or with sAJ4 (NK: sAJ4, 1:20) or with a combination of IL-2 (1000 U/ml) and sAJ3 (NK: sAJ3, 1:20) or with a combination of IL-2 (1000 U/ml) and sAJ4 (NK: sAJ4, 1:20) for 18 hours before the supernatants were harvested from NK cells to determine IFN-γ (FIG. 4A) and IL-10 (FIG. 4B) secretion using multiplex assay. NK cells were treated as described in FIG. 4A for 18 hours, and the number of cells secreting IFN-γ in the NK cells were determined as spot counts using ELISpot assay (FIG. 4C), and the supernatants were harvested to determine IFN-γ using single ELISA (FIG. 4D). NK cells were left untreated or treated with IL-2 (1000 U/ml) or with a combination of IL-2 (1000 U/ml) and anti-CD16 mAbs (3 μg/ml) or with a combination of IL-2 (1000 U/ml) and sAJ2 (NK: sAJ2, 1:20) or with a combination of IL-2 (1000 U/ml) and sAJ3 (NK: sAJ3, 1:20) or with a combination of IL-2 (1000 U/ml) and SAJ4 (NK: sAJ4, 1:20) or with sAJ2 (NK: sAJ2, 1:20) or with sAJ3 (NK: sAJ3, 1:20) or with SAJ4 (NK: sAJ4, 1:20) for 18 hours before the supernatants were harvested from NK cells to determine IFN-γ secretion using single ELISA. One of the representative experiments (FIG. 4E), and the combination of samples are shown (n=5) (FIG. 4F). NK cells were treated as described in FIG. 4E for 18 hours before the supernatant was harvested to determine IFN-γ secretion using multiplex assay (n=5) (FIG. 4G). NK were treated as described in FIG. 4E for 18 hours before the supernatants were harvested from PBMCs to determine IFN-γ and IL-10 secretion using specific single ELISAs, and ratio of IFN-γ to IL-10 was determined (FIG. 4H). NK were treated as described in FIG. 4E for 18 hours before the supernatants were harvested from PBMCs to determine IFN-γ and IL-10 secretion using multiplex assay, and ratio of IFN-γ to IL-10 was determined (n=4) (FIG. 4I). ***(p value <0.001), *(p value 0.01-0.05).

FIG. 5A-FIG. 5B show increased IFN-γ secretion levels in sAJ4 treated NK cells and monocytes in comparison to sAJ3 treated cells. NK cells and monocytes of healthy individuals were isolated from PBMCs as described in Example 4. NK cells or monocytes or co-culture of NK and monocytes (NK: monocytes; 1:1) treated with sAJ3 (NK: sAJ3, 1:20) or sAJ4 (NK: sAJ4, 1:20) or a combination of IL-2 (1000 U/ml) and sAJ3 (NK: sAJ3, 1:20) or a combination of IL-2 (1000 U/ml) and sAJ4 (NK: sAJ4, 1:20) or a combination of IL-2 (1000 U/ml) and anti-CD16 mAbs (3 μg/ml) and sAJ3 (NK: sAJ3, 1:20) or or a combination of IL-2 (1000 U/ml) and anti-CD16 mAbs (3 μg/ml) and sAJ4 (NK: sAJ4, 1:20) for 18 hours before the supernatants were harvested to determine IFN-γ using specific ELISA (FIG. 5A). NK cells and monocytes were treated as described in FIG. 5A for 18 hours before the supernatant was harvested to determine IFN-γ and IL-10 secretion using specific single ELISAs, and ratio of IFN-γ to IL-10 was determined (FIG. 5B).

FIG. 6 shows increased IFN-γ by sAJ4 treated CD8+ T cells in comparison to sAJ3 treated CD8+ T cells. CD8+ T cells were left untreated or treated with IL-2 (100 U/ml) or IL-2 (100 U/ml) and anti-CD3/28 mAbs (25 μl/ml), or IL-2 (100 U/ml) and sAJ2 (CD8+T: sAJ2, 1:20) or IL-2 (100 U/ml) and sAJ3 (CD8+ T: sAJ3, 1:20) or IL-2 (100 U/ml) and SAJ4 (CD8+ T: sAJ4, 1:20) for 18 hours before the supernatants were harvested from NK cells to determine IFN-γ using specific ELISAs.

FIG. 7 shows schematic representation of AJ3 function in ALS. AJ3 will be effective in alleviating auto-immunity, in particular in ALS since it will greatly regulate the levels and function of IFN-γ, decreasing over activation and death of motor neurons. Gene deletion or mutation may decrease MHC-class I expression on some motor neurons activating NK cells. Both cytokine and receptor mediated cross-linking will greatly sway the activation of the NK cells towards greater IFN-γ secretion in the presence of no or decreased IL-10. Induction of IFN-γ secretion by the NK cells will not only expand CD8+ T cells but also will differentiate and increase MHC-class I expression on motor neurons, allowing the mutated motor neurons to become susceptible to CD8+ T cell activation and effector function, in which case further IFN-γ secretion could exacerbate the death of not only mutated motor neurons, but also the non-mutated bystander cells, through over-activation and induction of cell death. Treatment with AJ3 will regulate the increase in the secretion of IFN-γ by increased induction of IL-10, decreasing the activation of NK cells and CD8+ T cells, and minimizing or even halting the death of motor neurons and slowing the progression of the disease. In the absence of disease, the default function of AJ3 formulation is towards increase in anti-inflammatory IL-10 induction.

### DETAILED DESCRIPTION OF THE INVENTION

The compositions and methods of the present disclosure are unique in their ability to differentially regulate production or secretion of various proinflammatory and anti-inflammatory cytokines by the NK cells. Accordingly, the compositions and methods of the present disclosure provide effective and safe therapies for patients afflicted with an inflammatory/autoimmune disease or cancer.

NK and T cells make up significant percentages of lymphocytes in peripheral blood mononuclear cells (PBMCs). NK cells are mainly known as the effectors of innate immunity due to their lack of antigen-specificity. CD4+ and CD8+ T cells mediate the adaptive cellular immunity, which closely collaborate with the innate immune system. NK cells and CD8+ T cells play significant role in cancer control, and NK and CD8+ T cell-based immunotherapies are among the leading standards in cancer therapeutics. Decreased function of these two lymphocytes result in poor prognosis in cancer patients. It has been shown that NK cells could activate and induce the proliferation of T cells, and also could kill chronically activated leukocytes. Increased levels of tumor-infiltrating CD8+ T cells were found to be associated with complete responses to standard chemotherapeutic regimens, and the presence of CD8+ memory T cells is associated with patient survival. It was found that NK cells can accelerate CD8+ T cells responses against viral infections, such as those caused by cytomegaloviruses.

AJ2 is a combination of different strains of gram-positive probiotic bacteria selected based on their superior ability to induce optimal secretion of both pro-inflammatory and anti-inflammatory cytokines in NK cells. Here, provided herein are evaluations of AI-Pro (AJ3), CA/I-Pro (AJ4), in addition to and incomparison to NK-CLK (AJ2). Sonicated bacteria (e.g., AJ2) was used for in-vitro studies and live bacteria (e.g., AJ2) for in-vivo studies, and similar effect on immune cells was seen. Contacting the NK cells with osteoclasts (OCs) and optional inclusion of sonicated AJ2 (sAJ2) gave rise to generate super-charged NK (sNK) cells with great potential to kill and differentiate tumors. The in-vivo studies have demonstrated that super-charged NK cells regulate the balance of T cell subsets, cytokine secretions, and cytotoxic activity of immune cells in various tissue compartments of mice. In addition, it was demonstrated that super-charged NK cells lyse activated CD4+ T and not CD8+ T cells, thus selecting and preferentially expanding CD8+ T cells.

Toll-like receptors (TLRs) are known to recognize common microbial patterns and, plays crucial role in innate immune response and initiation of adaptive immune response. NK cells were found to express TLR mRNA for TLR1-10. NK cells were found to produce higher levels of IFN-γ and also increased cytotoxic activity after TLR2, TLR3, TLR4 and TLR5 stimulation. TLRs 2, 4, 5 and 11 on the cell surface recognize bacterial lipoproteins, lipopolysaccharide (LPS), flagellin and profilin, respectively. TLRs 3, 7, 8 and 9 are expressed in endosomal compartments and recognize viral and bacterial nucleic acids. In addition, heat shock proteins (HSPs) and extracellular matrix components, such as fibronectin and hyaluronan can also activate the innate immune system through TLR2 and TLR4. Moreover, DNA complexes may activate TLR3, TLR7 and TLR9. TLR expression has been clearly shown on the surface of innate immune cells such as monocyte-macrophages and dendritic cells and therefore, TLRs have traditionally been considered to play an important role in indirectly controlling T cell responses through the activation of innate immune cells through TLRs. However, recent studies indicated that T cells can also express TLRs. At the moment it is not clear how TLRs contribute to the activation of T cells, however, the present disclosure indicates that functional modulation of T cells by probiotic bacteria, AJ3 and AJ4 is much less than that seen by the NK cells, even though there is still an increase in activation and regulation by the AJ4 and AJ3, respectively. Therefore, there are direct and indirect activation of T cells by AJ3 and AJ4 probiotic bacteria of T cells. Accordingly, in certain aspects, further provided herein are compositions comprising probiotic bacteria and methods of using them. In particular, the activities and functions of different formulations of probiotic bacteria (e.g., AJ2, AJ3, AJ4) have been explored. To compare the activation by the probiotic bacteria to either cytokine or receptor cross linking; PBMC, NK and CD8+ T cells were left untreated or treated with IL-2 or IL-2+anti-CD16mAbs or IL-2+anti-CD3/CD28mAbs in the presence and absence of SAJ2, sAJ3 and sAJ4. IL-2+anti-CD16mAbs activation of PBMCs and NK cells had the highest IFN-γ/IL-10 ratio whereas IL-2 combination with sAJ4 had the next highest followed by IL-2+sAJ2 and the lowest was seen with IL-2+sAJ3. Interestingly, IL-2+anti-CD3/CD28mAbs had lower IFN-γ/IL-10 in PBMCs when compared to either IL-2 alone or IL2+anti-CD16mAbs. Accordingly, the highest secretion of IFN-γ was seen when the PBMCs and NK cells were treated with IL-2+sAJ4, intermediate for IL-2+sAJ2 and the lowest IL-2+sAJ3. Indeed, IFN-γ secretion by IL-2+sAJ4 exceeded much higher than that of the levels of IFN-γ secretion by IL-2 or IL-2+anti-CD16mAbs or IL-2+anti-CD3/CD28mAbs in PBMCs and NK cells. The levels of IFN-γ secretion without IL-2 remained much lower and the highest was seen when sAJ4 was used to activate immune cells, and the ratio of IFN-γ to IL-10 remained much lower in all treatments when compared to those treated in the presence of IL-2. Of note is the difference between NK cells and CD8+ T cells in which synergistic induction of IFN-γ by IL-2+sAJ4 was significantly higher than those seen by CD8+ T cells indicating either lack of TLR receptors for response or augmented secretion of IL-10 which regulate the secretion of IFN-γ in CD8+ T cells. sAJ3 probiotic bacteria had the lowest IFN-γ/IL10 ratios and triggered much lower induction of IFN-γ, Thus, sAJ3 probiotic bacteria was formulated to augment anti-inflammatory cytokine IL-10 to counter the aggressive nature of pro-inflammatory cytokine IFN-γ induced by NK and CD8+ T cells in ALS patients.

Therefore, sAJ3 will alleviate auto-immunity seen in ALS by regulating the levels and function of IFN-γ, thereby decreasing overactivation and death of motor neurons.

By contrast, AJ4, which is particularly effective in activating NK cells and inducing IFN-g production, will treat cancer and cancer-related diseases.

### Definitions

The articles “a” and “an” are used herein to refer to one or to more than one (i.e. to at least one) of the grammatical object of the article. By way of example, “an element” means one element or more than one element.

The term “administering” is intended to include routes of administration which allow an agent to perform its intended function. Examples of routes of administration for treatment of a body which can be used include injection (subcutaneous, intravenous, parenterally, intraperitoneally, intrathecal, etc.), oral, inhalation, and transdermal routes. The injection can be bolus injections or can be continuous infusion. Depending on the route of administration, the agent can be coated with or disposed in a selected material to protect it from natural conditions which may detrimentally affect its ability to perform its intended function. The agent may be administered alone, or in conjunction with a pharmaceutically acceptable carrier. The agent also may be administered as a prodrug, which is converted to its active form in vivo.

The amount or level of cytokines and/or chemokines is “significantly” higher or lower than the normal amount of the cytokines and/or chemokines, if the amount is greater or less, respectively, than the normal level by an amount greater than the standard error of the assay employed to assess amount, and preferably at least about 10%, 20%, 30%, 40%, 50%, 60%, 70%, 80%, 90%, 100%, 150%, 200%, 300%, 350%, 400%, 500%, 600%, 700%, 800%, 900%, 1000% or more than that amount. Alternately, the amount of the cytokines and/or chemokines can be considered “significantly” higher or lower than the normal amount if the amount is at least about two, and preferably at least about three, four, or five times, higher or lower, respectively, than the normal amount of the cytokines and/or chemokines. Such “significance” can also be applied to any other measured parameter described herein, such as for expression, inhibition, cytotoxicity, cell growth, and the like.

The terms “conjoint therapy” and “combination therapy,” as used herein, refer to the administration of two or more therapeutic substances. The different agents comprising the combination therapy may be administered concomitant with, prior to, or following the administration of one or more therapeutic agents.

The term “control” refers to any reference standard suitable to provide a comparison to the expression products in the test sample. Such a control may comprise any suitable sample, including but not limited to a sample from a control diseased patient (e.g., those afflicted with an inflammatory/autoimmune disease or cancer) (can be stored sample or previous sample measurement) with a known outcome; normal tissue or cells isolated from a subject, such as a normal patient or the diseased patient (e.g., those afflicted with an inflammatory/autoimmune disease or cancer), cultured primary cells/tissues isolated from a subject such as a normal subject or the diseased patient (e.g., those afflicted with an inflammatory/autoimmune disease or cancer), adjacent normal cells/tissues obtained from the same organ or body location of the diseased patient, a tissue or cell sample isolated from a normal subject, or a primary cells/tissues obtained from a depository. In other embodiments, the control may comprise a reference standard cytokine/chemokine level from any suitable source, including but not limited to a previously determined cytokine/chemokine level range within a test sample from a group of patients, or a set of patients with a certain outcome (for example, survival for one, two, three, four years, etc.) or receiving a certain treatment (for example, standard of care cancer therapy). It will be understood by those of skill in the art that such control samples and reference standard cytokine/chemokine levels can be used in combination as controls in the methods of the present invention. In some embodiments, the control may comprise normal or non-diseased cell/tissue sample. In other embodiments, the control may comprise a level for a set of patients, such as a set of diseased patients (e.g., those afflicted with an inflammatory/autoimmune disease or cancer), or for a set of patients receiving a certain treatment, or for a set of patients with one outcome versus another outcome. In other preferred embodiments, the control may comprise normal cells, cells from patients treated with combination chemotherapy, and cells from patients having benign cancer. In still other embodiments, the control may also comprise a measured value for example, healthy or diseased individuals who were not treated with the compositions of the present disclosure, or healthy or diseased individuals who were administered with other probiotic compositions. In other embodiments, the control comprises a ratio of cytokine/chemokine levels, including but not limited to the level of one cytokine against the level of another cytokine, e.g., the ratio of the level of IFN-g and the level of IL-10. In particularly preferred embodiments, the control comprises a control sample which is of the same lineage and/or type as the test sample.

“Homologous” as used herein, refers to nucleotide sequence similarity between two regions of the same nucleic acid strand or between regions of two different nucleic acid strands. When a nucleotide residue position in both regions is occupied by the same nucleotide residue, then the regions are homologous at that position. A first region is homologous to a second region if at least one nucleotide residue position of each region is occupied by the same residue. Homology between two regions is expressed in terms of the proportion of nucleotide residue positions of the two regions that are occupied by the same nucleotide residue. By way of example, a region having the nucleotide sequence 5′-ATTGCC-3′ and a region having the nucleotide sequence 5′-TATGGC-3′ share 50% homology. Preferably, the first region comprises a first portion and the second region comprises a second portion, whereby, at least about 50%, and preferably at least about 75%, at least about 90%, or at least about 95% of the nucleotide residue positions of each of the portions are occupied by the same nucleotide residue. More preferably, all nucleotide residue positions of each of the portions are occupied by the same nucleotide residue.

The term “immune cell” refers to cells that play a role in the immune response. Immune cells are of hematopoietic origin, and include lymphocytes, such as B cells and T cells; natural killer cells; myeloid cells, such as monocytes, macrophages, eosinophils, mast cells, basophils, and granulocytes.

The term “immune response” refers to a response mediated by any or all immune cells. The “immune response” includes T cell mediated and/or B cell mediated immune responses. Exemplary immune responses include T cell responses, e.g., cytokine production and cellular cytotoxicity. In addition, the term immune response includes immune responses that are indirectly affected by T cell activation, e.g., antibody production (humoral responses) and activation of cytokine responsive cells, e.g., macrophages.

The term “immunotherapeutic agent” can include any molecule, peptide, antibody or other agent which can stimulate a host immune system to generate an immune response to a tumor or cancer in the subject. Various immunotherapeutic agents are useful in the compositions and methods described herein.

The term “inhibit” includes the decrease, limitation, or blockage, of, for example a particular action, function, or interaction. In some embodiments, cancer is “inhibited” if at least one symptom of the cancer is alleviated, terminated, slowed, or prevented. As used herein, cancer is also “inhibited” if recurrence or metastasis of the cancer is reduced, slowed, delayed, or prevented. Similarly, a biological function, such as the function of a protein, is inhibited if it is decreased as compared to a reference state, such as a control like a wild-type state.

A “kit” is any manufacture (e.g. a package or container) comprising at least one reagent, e.g. a composition (e.g., a pharmaceutical or nutraceutical composition) comprising at least one bacterial strain described herein. The kit may be promoted, distributed, or sold as a unit for performing the methods of the present invention. The kit may comprise one or more reagents necessary to produce a composition (e.g., cytokine/chemokine) useful in the methods of the present invention. In certain embodiments, the kit may also include instructional materials disclosing or describing the use of the kit. A kit may also include additional components to facilitate the particular application for which the kit is designed. A packaged pharmaceutical or nutraceutical composition may also be referred to as a kit.

The term “neoadjuvant therapy” refers to a treatment given before the primary treatment. Examples of neoadjuvant therapy can include chemotherapy, radiation therapy, and hormone therapy. For example, in treating breast cancer, neoadjuvant therapy can allows patients with large breast cancer to undergo breast-conserving surgery.

The term “preventing” is art-recognized, and when used in relation to a disease such as cancer or an inflammatory/autoimmune disease, is well understood in the art, and includes administration of a composition which reduces the frequency of, or delays the onset of, symptoms of a medical condition in a subject relative to a subject which does not receive the composition. Thus, prevention of cancer includes, for example, reducing the number of detectable cancerous growths in a population of patients receiving a prophylactic treatment relative to an untreated control population, and/or delaying the appearance of detectable cancerous growths in a treated population versus an untreated control population, e.g., by a statistically and/or clinically significant amount.

A “therapeutically effective amount” of a substance or cells is an amount capable of producing a medically desirable result in a treated patient, e.g., decrease tumor burden, decrease the growth of tumor cells, or alleviate any symptom associated with cancer or an inflammatory/autoimmune disease, with an acceptable benefit: risk ratio, preferably in a human or non-human mammal.

The term “treating” includes prophylactic and/or therapeutic treatments. The term “prophylactic or therapeutic” treatment is art-recognized and includes administration to the host of one or more of the subject compositions. If it is administered prior to clinical manifestation of the unwanted condition (e.g., disease or other unwanted state of the host animal), then the treatment is prophylactic (i.e., it protects the host against developing the unwanted condition); whereas, if it is administered after manifestation of the unwanted condition, the treatment is therapeutic (i.e., it is intended to diminish, ameliorate, or stabilize the existing unwanted condition or side effects thereof).

All numerical ranges provided herein are understood to be shorthand for all of the decimal and fractional values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50, as well as all intervening decimal values between the aforementioned integers such as, for example, 1.1, 1.2, 1.3, 1.4, 1.5, 1.6, 1.7, 1.8, and 1.9 and all intervening fractional values between the aforementioned integers such as, for example, ½, ⅓, ¼, ⅕, ⅙, ⅛, and 1/9, and all multiples of the aforementioned values. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

### Probiotic Bacteria

In some embodiments, the instant invention is drawn to a composition comprising at least one probiotic bacterial strain, capable of regulating NK cell function. Such probiotic bacteria induce significant production or secretion of various cytokines/chemokines, e.g., IFN-γ, Gro-alpha, IL-10, and TNF-α. In addition, such probiotic bacteria induce significant activation and/or expansion of NK cells.

Preferred probiotic bacteria species of the present disclosure include Streptococcus (e.g., S. thermophiles), Bifidobacterium (e.g., B. longum, B. breve, B. infantis, B. breve, B. infantis), and/or Lactobacillus genera (e.g., L. acidophilus, L. helveticus, L. bulgaricus, L. rhamnosus, L. plantarum, and L. casei). The compositions and methods of the present disclosure comprise at least one probiotic bacterial strain, preferably a combination of two or more different bacterial strains, to a subject, preferably a mammal (e.g., a human). Such administration may be systemically or locally (e.g., directly to intestines, e.g., orally or rectally) performed. The preferable administration route is oral administration. Other routes (e.g., rectal) may be also used. For administration, either the bacteria (e.g., in a wet, sonicated, ground, or dried form or formula), the bacterial culture medium comprising the bacteria, or the bacterial culture medium supernatant (not containing the bacteria), may be administered. The bacteria may be alive, partially alive, or dead. The bacteria may be sonicated, ground, wet, or dry (e.g., freeze-dried).

In some embodiments, the composition (bacterial, pharmaceutical, and/or nutraceutical) of the present disclosure comprises at least about 1×10⁴, 1×10⁵, 1×10⁶, 2×10⁶, 5×10⁶, 1×10⁷, 5×10⁷, 1×10⁸, 5×10⁸, 10×10⁸, 100×10⁸, 1×10⁹, 5×10⁹, 10×10⁹, 100×10⁹, 110×10⁹, 120×10⁹, 130×10⁹, 140×10⁹, 150×10⁹, 160×10⁹, 170×10⁹, 180×10⁹, 190×10⁹, 200×10⁹, 210×10⁹, 220×10⁹, 230×10⁹, 240×10⁹, 250×10⁹, 260×10⁹, 270×10⁹, 280×10⁹, 290×10⁹, 300×10⁹, 310×10⁹, 320×10⁹, 330×10⁹, 340×10⁹, 350×10⁹, 360×10⁹, 370×10⁹, 380×10⁹, 390×10⁹, 400×10⁹, 410×10⁹, 420×10⁹, 430×10⁹, 440×10⁹, 450×10⁹, 460×10⁹, 470×10⁹, 480×10⁹, 490×10⁹, or 500×10⁹, but no more than 510×10⁹, 520×10⁹, 530×10⁹, 540×10⁹, 550×10⁹, 600×10⁹, 650×10⁹, 700×10⁹, 750×10⁹, 800×10⁹, 850×10⁹, 900×10⁹, 950×10⁹, or 1000×10⁹ total CFU of bacteria per gram of the composition.

In some embodiments, the composition comprises at least about 180×10⁹ but no more than about 270×10⁹ total CFU of bacteria per gram of the composition. In preferred embodiments, the composition comprises about 250×10⁹ total CFU of bacteria per gram of the composition.

### AJ3 Composition

In some embodiments, the composition (bacterial, pharmaceutical, and/or nutraceutical) of the present disclosure comprises at least two bacterial strains selected from: Bifidobacterium longum, Bifidobacterium breve, and Bifidobacterium infantis. In some embodiments, one or more bacterial strains are intact. In some embodiments, one or more bacterial strains are sonicated. In preferred embodiments, the composition is an AJ3 composition comprising Bifidobacterium longum, Bifidobacterium breve, and Bifidobacterium infantis.

In some embodiments, at least about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, or 70%, but no more than about 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% of the bacteria in the composition are Bifidobacterium Longum.

In some embodiments, at least about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, or 50%, but no more than about 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% of the bacteria in the composition are Bifidobacterium breve.

In some embodiments, at least about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, or 70%, but no more than about 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% of the bacteria in the composition are Bifidobacterium infantis.

In some embodiments, the percent bacteria refers to the percentage of the colony forming unit (CFU) of said bacteria relative to the total CFU of bacteria in the composition.

In some embodiments, the bacteria in the composition comprise about 50% (or about 40% to about 60%) Bifidobacterium Longum, about 10% (or about 1% to about 20%) Bifidobacterium breve, and about 40% (or about 30% to about 50%) Bifidobacterium infantis, wherein the percent bacteria refers to the percentage of the CFU of said bacteria relative to the total CFU of bacteria in the composition.

### AJ4 Composition

In some embodiments, the composition (bacterial, pharmaceutical, and/or nutraceutical) of the present disclosure comprises at least two bacterial strains selected from: Streptococcus thermophiles, Lactobacillus acidophilus, Lactobacillus plantarum, and Lactobacillus paracasei. In some embodiments, one or more bacterial strains are intact. In some embodiments, one or more bacterial strains are sonicated. In preferred embodiments, the composition is an AJ4 composition comprising Streptococcus thermophiles, Lactobacillus acidophilus, Lactobacillus plantarum, and Lactobacillus paracasei.

In some embodiments, at least about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, or 60%, but no more than about 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% of the bacteria in the composition are Streptococcus thermophiles.

In some embodiments, at least about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, or 50%, but no more than about 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% of the bacteria in the composition are Lactobacillus acidophilus.

In some embodiments, at least about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, or 70%, but no more than about 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% of the bacteria in the composition are Lactobacillus plantarum.

In some embodiments, at least about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, or 40%, but no more than about 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% of the bacteria in the composition are Lactobacillus paracasei.

In some embodiments, the percent bacteria refers to the percentage of the colony forming unit (CFU) of said bacteria relative to the total CFU of bacteria in the composition.

In some embodiments, the bacteria in the composition comprise about 30% (or about 20% to about 40%) Streptococcus thermophiles, about 20% (or about 10% to about 30%) Lactobacillus acidophilus, about 40% (or about 30% to about 50%) Lactobacillus plantarum, and about 10% (or about 1% to about 20%) Lactobacillus paracasei, wherein the percent bacteria refers to the percentage of the CFU of said bacteria relative to the total CFU of bacteria in the composition.

### AJ2 Composition

AJ2 is a combination of 8 different strains of gram positive probiotic bacteria which have been cloned to withstand high temperature and also low pH (Streptococcus thermophiles, Bifidobacterium longum, Bifidobacterium breve, Bifidobacterium infantis, Lactobacillus acidophilus, Lactobacillus plantarum, KE99, and Lactobacillus paracasei). The AJ2 composition lacking KE99 was shown to be equivalent to AJ2. AJ2 has the ability to induce synergistic production of IFN-γ when added to IL-2-treated or IL-2+anti-CD16 monoclonal antibody-treated NK cells (anti-CD16mAb). The combination of strains was used to provide bacterial diversity in addition to synergistic induction of a balanced pro and anti-inflammatory cytokine and growth factor release in NK cells. Moreover, the quantity of each bacteria within the combination of strains was adjusted to yield a closer ratio of IFN-γ to IL-10 to that obtained when NK cells are activated with IL-2+anti-CD16mAb in the absence of bacteria. The rationale behind such selection was to obtain a ratio similar to that obtained with NK cells activated with IL-2+anti-CD16mAb in the absence of bacteria since such treatment provided significant differentiation of the cells.

### Cytokines

Cytokines include a broad and loose category of small proteins (˜5-20 kDa) that are important in cell signaling. Their release has an effect on the behaviour of cells around them. cytokines are involved in autocrine signalling, paracrine signaling and endocrine signalling as immunomodulating agents. Cytokines include chemokines, interferons, interleukins, lymphokines, and tumour necrosis factors, and may additionally include hormones or growth factors in the instant disclosure. Cytokines are produced by a broad range of cells, including immune cells like macrophages, B lymphocytes, T lymphocytes and mast cells, as well as endothelial cells, fibroblasts, and various stromal cells. Preferred cytokines are exemplified in the specification and the Tables of the instant disclosure.

The term “cytokine/chemokine activity,” includes the ability of a cytokine or a chemokine to modulate at least on of cellular functions. Generally, cytokines or chemokines modulate the balance between humoral and cell-based immune responses, and they regulate the maturation, growth, and responsiveness of particular cell populations. Thus, the term “cytokine/chemokine activity” includes the ability of a cytokine or chemokine to bind its natural cellular receptor(s), the ability to modulate cellular signals, and the ability to modulate the immune response.

Cytokines that play a major role in the innate immune system include: TNF-α, IL-1, IL-10, IL-12, IFN-α, IFN-β, IFN-γ, and chemokines. TNF-α is an important mediator of acute inflammation. It mediates the recruitment of macrophages and neutrophils to sites of infection by stimulating endothelial cells to produce adhesion molecules and by producing chemotactic cytokines-chemokines. TNF-α also acts on the hypothalamus to produce fever and it promotes the production of acute phase proteins. Interleukin 1 is another inflammatory cytokine produced by activated macrophages. It promotes activation, costimulation, and secretion of cytokines and other acute-phase proteins. Interleukin 10 is produced by activated macrophages, B cells, and T helper cells. It is predominantly an inhibitory cytokine. Interleukin 10 decreases antigen presentation and the expression of class II MHC and co-stimulatory molecules on macrophages, resulting in a dampening of immune responses. Interleukin 12 is produced by activated macrophages, B cells and dendritic cells. It induces the differentiation of Th cells to become Th1 cells and enhances the cytolytic functions of T cytotoxic cells and NK cells. Interferon-alpha and interferon-beta are cytokines produced by macrophages, dendritic cells, and many other cell types. They promote resistance to viral pathogens and promote increased expression of MHC class I. Interferon gamma is an important cytokine produced by Th1 cells and NK cells. It promotes activation of APCs and cell-mediated immunity, and increase expression of MHC class II molecules. IFN gamma has numerous functions in both innate immune and adaptive immune systems. Chemokines are chemotactic cytokines produced by many kinds of leukocytes and other cell types. They represent a large family of molecules that function to recruit leukocytes to sites of infection and play a role in lymphocyte trafficking.

Another group of cytokines that play a critical role in adaptive immune system include IL-2, IL-4, IL-5, TGF-β, IL-10 and IFN-γ. Interleukin 2 is a type I cytokine produced primarily by T helper cells. It is the major growth factor for T cells. It also promotes the growth of B cells and can activate NK cells and monocytes. Interleukin 4 is produced by T cells and mast cells. It stimulates proliferation and differentiation of Th2 cells, while inhibit Th17 development. Interleukin 4 also stimulates Ig class switching to the IgE isotype. Interleukin 5 is a cytokine produced by Th2 cells. It functions to promote the growth and differentiation of B cells and eosinophiles. Transforming growth factor beta is a cytokine and also is a growth factor produced by T cell, macrophages, and many other cell types. It is primarily an inhibitory cytokine, which inhibits the proliferation of T cells and blocks the effects of pro-inflammatory cytokines.

### Exemplary Cytokines in Inflammatory Diseases

An inflammatory cytokine or proinflammatory cytokine is a type of signaling molecule (a cytokine) that is secreted from immune cells like helper T cells (Th) and macrophages, and certain other cell types that promote inflammation. They include interleukin-1 (IL-1), IL-12, and IL-18, tumor necrosis factor alpha (TNF-α), interferon gamma (IFNγ), and granulocyte-macrophage colony stimulating factor (GM-CSF) and play an important role in mediating the innate immune response. Inflammatory cytokines are predominantly produced by and involved in the upregulation of inflammatory reactions.

Excessive chronic production of inflammatory cytokines contribute to inflammatory diseases, that have been linked to different diseases, such as atherosclerosis and arthritis. Dysregulation has also been linked to depression and other neurological diseases. A balance between proinflammatory and anti-inflammatory cytokines is necessary to maintain health.

IL-10 is a homodimeric cytokine of 17 kDa that was discovered as a potent inhibitor of macrophage effector functions. It is produced by activated monocytes, NK cells, B cells and T cells. In human, IL-10 can be produced by both the Th1 and Th2 subsets. IL-10 is able to ameliorate potential pathological autoimmune inflammation through the inhibition at various facets of the immune response. IL-10 inhibits the production of pro-inflammatory cytokines by macrophages, such as IL-1B, IL-6, IL-8, IL-12 and THF-α, and upregulates the production if IL-1Ra and soluble p55 and p75 TNF-receptors.

In T cells, IL-10 inhibits production of IL-2 and IFN-γ and also block T cell proliferation. Thus, IL-10 has potent anti-inflammatory functions and has, consequently, been used in the treatment of experimental autoimmune disease with great success. IL-4 and IL-10 synergistically reduce joint inflammation in acute and chronic arthritis models. Thus, IL-10 might be an effective means for downregulating human chronic autoimmune inflammation by counteracting IFN-γ mediated pro-inflammatory activities.

The chemokine (C-X-C motif) ligand 1 (CXCL1) is a small peptide belonging to the CXC chemokine family that acts as a chemoattractant for several immune cells, especially neutrophils or other non-hematopoietic cells to the site of injury or infection and plays an important role in regulation of immune and inflammatory responses. It is also called GROα or Gro-alpha. It was recently demonstrated that Gro-alpha prevents or treats inflammatory diseases. For example, administration of Gro-alpha abrogated autoimmune inflammatory heart disease (see e.g., Bachmaier et al (2014) PLOS One 9 (6): e100608).

Granulocyte colony-stimulating factor (G-CSF), an endogenous hematopoietic growth factor for neutrophils produced at the site of infection, is an integral part of this natural host defense. However, neutrophilic granulocytes also play a major role in the inflammatory response, resulting in tissue damage. Therefore, the indications for colony-stimulating factors in combating infectious diseases seemed to be limited for fear of their proinflammatory activity. In contrast to these concerns, G-CSF has proven itself to be an anti-inflammatory immunomodulator. Animal, volunteer, and patient studies have all shown that G-CSF reduces inflammatory activity by inhibiting the production or activity of the main inflammatory mediators interleukin-1, tumor necrosis factor-alpha, and interferon gamma. In conclusion, the body's G-CSF-regulated emergency recruitment of neutrophils is combined with a simultaneous limitation of the harmful inflammatory reaction (see Hartung (1998) Curr Opin Hematol, 5 (3): 221-225).

### Interfereon-Gamma (IFN-γ) and its Antitumor Effects

### IFN-γ Induces Apoptosis of Cancer Cells

It has been demonstrated that high doses of IFN-γ could induce apoptosis in NSCLC cell-lines, namely A549 and H460, by activating JAK-STAT1-caspase signaling. Western blot analyses showed that STAT1 forced transcription and synthesis of caspase 3 and caspase 7, which further initiated apoptotic processes in cancer cells (Song et al. (2019) Cancer Res, 81771781:1-29. Additionally, it was shown that IFN-γ can increase the motility of antigen-specific CD8+ T-cells to the antigen-expressing (target) cells and enhance the killing capacity of target cells. When IFN-γ+/+ and IFN-γ−/− CD8 T-cells were incubated with the target cells, significantly higher effectiveness of IFN-γ competent cells was observed. Addition of anti-IFN-γ-antibody to the co-culture system markedly reduced target cell killing. Interestingly, IFN-γ can selectively induce apoptosis in stem-like colon cancer cells through JAK-STAT1-IRF1 signaling in a dose-dependent manner. Kundu et al. reported that precise neutralization of cytokine from IL-12 family, namely p40 monomer, induces IL-12-IFN-γ signaling cascade in prostate cancer both in vitro and in vivo, which subsequently leads to cancer cells death and tumor regression. They found that anti-p40 antibody treatment significantly elevated the expression of apoptosis-related genes such as caspase 3, caspase 7, caspase 8, caspase 9, BAD, BID, cytochrome C, BAK, and p53 (Kundu et al. (2017) PNAS 114 (43): 11482-7). Consistently, in NSCLC cells lines, namely H1975, HCC827, and H1437, IFN-γ induced programmed cell death through the activation of caspases downstream of JAK-STAT1 signaling. Similar results have been reported in melanoma cells wherein the activation of caspase 3 was IFN-γ/IRF3/ISG54 dependent.

### Other IFN-γ-Dependent Tumor-Suppressive Mechanisms

Although, IFN-γ can directly affect the viability of tumor cells, increasing evidence points to interactions with surrounding stromal cells for effective rejection of solid tumors. For instance, immunohistology analyses of large tumor sections revealed that IFN-γ could reduce the number of endothelial cells and induce blood vessel destruction and later promote tumor tissue necrosis. In fact, Kammertoens et al. showed responsiveness of cancer endothelial cells by highlighting the necessary role that IFN-γ plays in the regression of solid tumors. By using electron microscopy they observed that IFN-γ-exposed endothelial cells became round, condensed, and more occluded, which reduced blood flow in tumor tissues and subsequently, prompted tumor ischemia (Kammertoen et al. (2017) Nature 2017; 545:98-102). Similarly, by interacting with stromal fibroblasts IFN-γ downregulated the expression of vascular endothelial growth factor A, a growth factor critical for tumor neovascularization. Therefore, it is equally important to investigate IFN-γ-mediated effects on tumor stromal cells, especially in solid, well-established tumors.

Interplay between IFN-γ and macrophages in an inflamed setting has previously been described and has raised questions regarding their interaction in the tumor microenvironment (TME). Unsurprisingly, crosstalk between IFN-γ and M1-like immunostimulatory tumor-associated macrophages (TAMs) was sufficient to inhibit tumor growth in Lewis lung carcinoma and colon adenocarcinoma. Generated M1-like TAMs secreted CXCL9, CXCL10, and CD86, which stimulated the recruitment of cytotoxic T lymphocytes (CTLs) to the TME as well as their activation. Recruited CTLs produced IFN-γ that was proven to be critical for sustaining M1 TAM activity and tumor inhibition. Reprograming of IL33−/− Tregs was also linked to higher IFN-γ production and thus, improved the immune response in tumor tissue.

IFN-γ interacts with distinct cytokines from the TME to induce cancer growth arrest. Synergistically with TNF, IFN-γ stimulates the senescence of tumor cell growth through stabilization of p16INK4a—Rb pathway. This effect is mediated by activation of STAT1 and TNF receptor 1 and is maintained permanently in vitro and in vivo. Together with inducing apoptosis or senescence, IFN-γ can shift tumors to a dormant state. As recently shown IFN-γ—mediated upregulation of IDO1 increased the intracellular concentration of kynurenine (kyn, IDO1—catalyzed tryptophan metabolite), which activated aryl hydrocarbon receptor (AhR). AhR moved to the nucleus and directly upregulated transcription of cell cycle-regulatory molecule, p27. Thus, IDO1-Kyn-AhR-p27 pathway was proposed as a mechanism which explains how high concentration of IFN-γ induces tumor dormancy. The existence of IL-12-IFN-γ relationship has also been described. As the IL-12 producers, dendritic cells (DCs) stimulate NK cells to secrete IFN-γ, therefore, survival of tumor-bearing mice was improved and number of metastasis was reduced. Moreover, IFN-γ produced by NK cells altered tumor structure and limited the number of metastasis by increasing the expression of the extracellular matrix protein, fibronectin 1 (Glasner et al. (2018) Immunity 48:107-19).

### IFN-γ Contributes to the Efficiency of Cancer Immunotherapy

The discovery of antibodies targeting immune checkpoint molecules, such as programmed cell death protein 1 (PD-1), its ligand PD-L1, and cytotoxic T-lymphocyte-associated protein 4 (CTLA-4), provided hope for patients with chemo-resistant and late-stage tumors. However, their efficiency has only been proven in a small portion of treated patients. IFN-γ is believed to be one of the critical factors determining the success of immunotherapy. By analyzing gene expression profiles from tumor tissue samples, Ayers et al. reported that metastatic melanoma, head and neck squamous cell carcinoma, and gastric cancer patients who responded to anti-PD-1 therapy had higher expression scores for IFN-γ-related genes when compared to non-responders. They proposed that the detected IFN-γ signature (IDO1, CXCL10, CXCL9, HLA-DRA, STAT1, and IFNG) can be a prediction marker for the clinical response to immune checkpoint inhibitors (Ayers et al. (2017). J Clin Invest. 127 (8): 2930-40). Similarly, a four-gene IFN-γ signature (IFNG, CD274, LAG3, and CXCL9) has been suggested as identifying pattern for urothelial and NSCLC patients who can benefit from the anti-PD-L1 antibody durvalumab. Moreover, successful anti-PD-1 treatment depends on intratumoral crosstalk between IL-12 and IFN-γ. After binding to PD-1, this antibody stimulates CD8+ T-cells to secrete IFN-γ, which activates its receptor on DCs, thus increasing the production of IL-12 in the TME. The newly generated interleukin acts back on CD8+ T cells to further stimulate IFN-γ production and enhance cytotoxic tumor cell function. Therefore, a combination of anti-PD-1 antibody and induction of INF-γ via the compositions of the present disclosure would be particularly useful in preventing and/or treating cancer. Alternative mechanism by which IFN-γ contributes to efficiency of cancer immunotherapy was described by Wang et al. In that model, tumor-infiltrating CD8+ T-cells secreted IFN-γ in response to nivolumab, an anti-PD-L1 antibody. The released IFN-γ mediated lipid peroxidation and ferroptosis in tumor cells by reducing the uptake of cystine and excretion of glutamate, resulting in tumor cells death both in vitro and in vivo. Mechanistically, type II interferon activated the JAK1-STAT1 signaling pathway, which further downregulated the transcription of SLC7A11 and SLC3A2 proteins of the glutamate-cystine antiporter system. Likewise, the clinical benefits of cancer immunotherapy were reduced in nivolumab-treated mice bearing INFGR−/− tumors.

Thibaut et al. recently suggested a model in which tumor-reactive T-cells secrete IFN-γ, which diffuses extensively to alter the TME in distant areas. The prolonged activity of IFN-γ has been shown to be crucial for antitumor immune response as shown by induction of PD-L1 expression and inhibition of tumor growth (Hoekstra et al. (2020) Nat Cancer 1 (3): 291-301). Furthermore, Zhang et al. proposed that IFN-γ may be a good therapeutic option for improving the efficacy of PD-1 blockade therapy for pancreatic cancer by preventing the trafficking of CXCR2+CD68+ immunosuppressive macrophages to the TME by blocking the CXCL8-CXCR2 axis (Zhang et al. (2020) J Immunother Cancer 8 (1): 1-15).

The efficiency of anti-CTLA-4 therapy was also IFN-γ dependent. Whole exome sequencing data showed that melanoma tumors resistant to immunotherapy had defects in IFN-γ signaling, namely loss of IFNGR1, IRF-1, JAK2 and IFNGR2 genes, as well as amplification of SOCS1 and PIAS4 inhibitory genes. Therefore, the combination of immune checkpoint inhibitors and IFN-γ can be a good strategy to increase the overall efficiency of cancer immunotherapy. Indeed, two such clinical trials have already been initiated testing the combination of nivolumab or pembrolizumab with IFN-γ (NCT02614456 and NCT03063632, respectively). Other studies suggest that disruption of IFN-γ signaling in tumor cells could boost tumor growth and impact the efficiency of given immune checkpoint inhibitor therapy. Amplification of IFN-γ-pathway inhibitory molecules or downregulation and loss of its receptor and downstream signaling mediators are common mechanisms for tumor cells to avoid generated immune response. It was recently shown that aging can also consistently attenuate IFN-γ signaling in triple-negative breast cancer patients and limit the efficiency of immune checkpoint blockade (ICB) therapy. Another hypothesis is that enhanced intratumoral production of IFN-γ can improve the potency of ICB therapy in patients with cancer. For example, pharmacological blockade or partial genetic deletion of CBM complex (CARMA1-BCL10-MALT1) in Tregs re-program them to secrete IFN-γ which results in tumor regression. In addition, combination of CBM inhibition and anti-PD-1 antibodies enabled tumor control in MC38 colon carcinoma-bearing mice who were resistant to anti-PD-1 monotherapy. Similarly, tumor regression has been observed only in melanoma-bearing mice treated with PD-1 targeted therapy together with antibodies against neuropilin-1. Neuropilin-1 is a protein found on most of the tumor-infiltrating Tregs, important for their suppressive function. Notably, Neuropilin-1 deletion in Tregs led to increased expression of Th1 cell markers such as T-bet and IFN-γ. Treg-secreted IFN-γ drove intratumoral fragility of the remaining immune-suppressive Tregs via hypoxia-inducible factor 1-alpha (HIF1α) which stimulated host immunity to eliminate cancer cells. Similarly, it was suggested that metastatic potential of tumor cells after receiving immunotherapy was due to reduction of IFN-γ in the TME and augmented activity of integrin αvβ3 signaling axis. Collectively, the presence of IFN-γ in the TME is required for optimal antitumor responses in cancer patients receiving mono- or combined immune checkpoint inhibitors (see e.g., Jorgovanovic et al. (2020) Biomarker Research 9:49 and references therein).

### Osteoclasts

Osteoclasts are a type of bone cell, derived from hematopoietic stem cells. Their function, resorbing bone tissue, is critical for the maintenance, repair, and remodeling of bones. Bone homeostasis is achieved when there is a balance between osteoblast bone formation and osteoclast bone resorption. Osteoclasts mature through stimulation from osteoblasts expressing RANKL, and their interaction, mediated by firm adhesion via ICAM-1. Osteoclasts also express many ligands for receptors present on activated NK cells. They reported that osteoclasts express ULBP-1, ULBP-2/5/6 and ULBP-3, but little or no MIC-A, MIC-B, or MHC class I-like ligands for NKG2D, the activating receptor of NK cells.

Osteoclasts (OCs), in comparison to dendritic cells (DCs) and monocytes, are significant activators of NK cell expansion and function (Tseng et al. (2015) Oncotarget 6 (24): 20002-25). Additionally, osteoclasts secrete significant amounts of IL-12, IL-15, IFN-γ and IL-18, which are known to activate NK cells; osteoclasts also express important NK-activating ligands. Accordingly, osteoclasts expand and activate NK cells to levels that are higher than those established by other methodologies.

### Methods for Detecting Cytokines or Chemokines

The activity or level of a cytokine or chemokine can be detected and/or quantified by detecting or quantifying the expressed polypeptide. The polypeptide can be detected and quantified by any of a number of means well-known to those of skill in the art. Accordingly, any method known in the art for detecting polypeptides can be used. Such methods include, but are not limited to, immunodiffusion, immunoelectrophoresis, radioimmunoassay (RIA), enzyme-linked immunosorbent assays (ELISAs), immunofluorescent assays, Western blotting, binder-ligand assays, immunohistochemical techniques, agglutination, complement assays, high performance liquid chromatography (HPLC), thin layer chromatography (TLC), hyperdiffusion chromatography, and the like (e.g., Basic and Clinical Immunology, Sites and Terr, eds., Appleton and Lange, Norwalk, Conn. pp 217-262, 1991 which is incorporated by reference). Preferred are binder-ligand immunoassay methods including reacting antibodies with an epitope or epitopes and competitively displacing a labeled polypeptide or derivative thereof.

For example, ELISA and RIA procedures may be conducted such that a desired cytokine/chemokine standard is labeled (with a radioisotope such as ¹²⁵I or ³⁵S, or an assayable enzyme, such as horseradish peroxidase or alkaline phosphatase), and, together with the unlabeled sample, brought into contact with the corresponding antibody, whereon a second antibody is used to bind the first, and radioactivity or the immobilized enzyme assayed (competitive assay). Alternatively, the biomarker protein in the sample is allowed to react with the corresponding immobilized antibody, radioisotope- or enzyme-labeled anti-biomarker protein antibody is allowed to react with the system, and radioactivity or the enzyme assayed (ELISA-sandwich assay). Other conventional methods may also be employed as suitable.

The above techniques may be conducted essentially as a “one-step” or “two-step” assay. A “one-step” assay involves contacting antigen with immobilized antibody and, without washing, contacting the mixture with labeled antibody. A “two-step” assay involves washing before contacting, the mixture with labeled antibody. Other conventional methods may also be employed as suitable.

In certain embodiments, a method for measuring the cytokine/chemokine levels comprises the steps of: contacting a biological specimen with an antibody or variant (e.g., fragment) thereof which selectively binds the biomarker protein, and detecting whether said antibody or variant thereof is bound to said sample and thereby measuring the levels of the biomarker protein.

Enzymatic and radiolabeling of biomarker protein and/or the antibodies may be effected by conventional means. Such means will generally include covalent linking of the enzyme to the antigen or the antibody in question, such as by glutaraldehyde, specifically so as not to adversely affect the activity of the enzyme, by which is meant that the enzyme must still be capable of interacting with its substrate, although it is not necessary for all of the enzyme to be active, provided that enough remains active to permit the assay to be effected. Indeed, some techniques for binding enzyme are non-specific (such as using formaldehyde), and will only yield a proportion of active enzyme.

It is usually desirable to immobilize one component of the assay system on a support, thereby allowing other components of the system to be brought into contact with the component and readily removed without laborious and time-consuming labor. It is possible for a second phase to be immobilized away from the first, but one phase is usually sufficient.

It is possible to immobilize the enzyme itself on a support, but if solid-phase enzyme is required, then this is generally best achieved by binding to antibody and affixing the antibody to a support, models and systems for which are well-known in the art. Simple polyethylene may provide a suitable support.

Enzymes employable for labeling are not particularly limited, but may be selected from the members of the oxidase group, for example. These catalyze production of hydrogen peroxide by reaction with their substrates, and glucose oxidase is often used for its good stability, ease of availability and cheapness, as well as the ready availability of its substrate (glucose). Activity of the oxidase may be assayed by measuring the concentration of hydrogen peroxide formed after reaction of the enzyme-labeled antibody with the substrate under controlled conditions well-known in the art.

Other techniques may be used to detect biomarker protein according to a practitioner's preference based upon the present disclosure. One such technique is Western blotting (Towbin et at., Proc. Nat. Acad. Sci. 76:4350 (1979)), wherein a suitably treated sample is run on an SDS-PAGE gel before being transferred to a solid support, such as a nitrocellulose filter. Anti-biomarker protein antibodies (unlabeled) are then brought into contact with the support and assayed by a secondary immunological reagent, such as labeled protein A or anti-immunoglobulin (suitable labels including ¹²⁵I, horseradish peroxidase and alkaline phosphatase). Chromatographic detection may also be used.

Immunohistochemistry may be used to detect expression of biomarker protein, e.g., in a biopsy sample. A suitable antibody is brought into contact with, for example, a thin layer of cells, washed, and then contacted with a second, labeled antibody. Labeling may be by fluorescent markers, enzymes, such as peroxidase, avidin, or radiolabeling. The assay is scored visually, using microscopy.

Antibodies that may be used to detect biomarker protein include any antibody, whether natural or synthetic, full length or a fragment thereof, monoclonal or polyclonal, that binds sufficiently strongly and specifically to the biomarker protein to be detected. An antibody may have a K_{d} of at most about 10⁻⁶M, 10⁻⁷M, 10⁻⁸M, 10⁻⁹M, 10⁻¹⁰M, 10⁻¹¹M, 10⁻¹²M. The phrase “specifically binds” refers to binding of, for example, an antibody to an epitope or antigen or antigenic determinant in such a manner that binding can be displaced or competed with a second preparation of identical or similar epitope, antigen or antigenic determinant. An antibody may bind preferentially to the biomarker protein relative to other proteins, such as related proteins.

Antibodies may be commercially available or may be prepared according to methods known in the art.

Antibodies and derivatives thereof that may be used encompass polyclonal or monoclonal antibodies, chimeric, human, humanized, primatized (CDR-grafted), veneered or single-chain antibodies as well as functional fragments, i.e., biomarker protein binding fragments, of antibodies. For example, antibody fragments capable of binding to a biomarker protein or portions thereof, including, but not limited to, Fv, Fab, Fab′ and F(ab′)₂ fragments can be used. Such fragments can be produced by enzymatic cleavage or by recombinant techniques. For example, papain or pepsin cleavage can generate Fab or F(ab′)₂ fragments, respectively. Other proteases with the requisite substrate specificity can also be used to generate Fab or F(ab′)₂ fragments. Antibodies can also be produced in a variety of truncated forms using antibody genes in which one or more stop codons have been introduced upstream of the natural stop site. For example, a chimeric gene encoding a F(ab′)₂ heavy chain portion can be designed to include DNA sequences encoding the CH, domain and hinge region of the heavy chain.

In some embodiments, agents that specifically bind to a cytokine/chemokine other than antibodies are used, such as peptides. Peptides that specifically bind to a cytokine/chemokine is well known in the art (e.g., receptor fragment for the cytokine/chemokine), and can also be identified by any means known in the art. For example, specific peptide binders of a biomarker protein can be screened for using peptide phage display libraries.

### Sampling Methods

In some embodiments, cytokine/chemokine amount and/or activity measurement(s) in a sample from a subject is compared to a predetermined control (standard) sample. The sample from the subject is typically from blood or tissue. The control sample can be from the same subject or from a different subject. The control sample is typically a normal, non-diseased sample. However, in some embodiments, such as for staging of disease or for evaluating the efficacy of treatment, the control sample can be from a diseased subject. The control sample can be a combination of samples from several different subjects. In some embodiments, the cytokine/chemokine amount and/or activity measurement(s) from a subject is compared to a pre-determined level. This pre-determined level is typically obtained from normal samples. As described herein, a “pre-determined” cytokine/chemokine amount and/or activity measurement(s) may be a cytokine/chemokine amount and/or activity measurement(s) used to, by way of example only, evaluate a subject that may be selected for treatment, evaluate a response to a composition as disclosed herein, alone or in combination with other immunotherapies and with one or more additional anti-cancer therapies or anti-inflammation therapies. A pre-determined cytokine/chemokine amount and/or activity measurement(s) may be determined in populations of patients with or without a disease (e.g., cancer or inflammatory/autoimmune disease). The pre-determined cytokine/chemokine amount and/or activity measurement(s) can be a single number, equally applicable to every patient, or the pre-determined cytokine/chemokine amount and/or activity measurement(s) can vary according to specific subpopulations of patients. Age, weight, height, and other factors of a subject may affect the pre-determined cytokine/chemokine amount and/or activity measurement(s) of the individual. Furthermore, the pre-determined cytokine/chemokine amount and/or activity can be determined for each subject individually. In some embodiments, the amounts determined and/or compared in a method described herein are based on absolute measurements.

In other embodiments, the amounts determined and/or compared in a method described herein are based on relative measurements, such as ratios (e.g., cytokine/chemokine level, and/or activity before a treatment vs. after a treatment, and the like). For example, the relative analysis can be based on the ratio of pre-treatment cytokine/chemokine measurement as compared to post-treatment cytokine/chemokine measurement. Pre-treatment cytokine/chemokine measurement can be made at any time prior to initiation of anti-cancer therapy or an anti-inflammation therpay. Post-treatment cytokine/chemokine measurement can be made at any time after initiation of anti-cancer therapy or an anti-inflammation therapy. In some embodiments, post-treatment cytokine/chemokine measurements are made 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20 weeks or more after initiation of the administration of the compositions of the present disclosure.

In some embodiments of the present invention the change of cytokine/chemokine amount and/or activity measurement(s) from the pre-determined level is about 0.1, 0.2, 0.3, 0.4, 0.5, 0.6, 0.7, 0.8, 0.9, 1.0, 1.5, 2.0, 2.5, 3.0, 3.5, 4.0, 4.5, or 5.0 fold or greater, or any range in between, inclusive. Such cutoff values apply equally when the measurement is based on relative changes, such as based on the ratio of pre-treatment cytokine/chemokine measurement as compared to post-treatment cytokine/chemokine measurement.

Biological samples can be collected from a variety of sources from a patient including a body fluid sample, cell sample, or a tissue sample. “Body fluids” refer to fluids that are excreted or secreted from the body as well as fluids that are normally not (e.g., amniotic fluid, aqueous humor, bile, blood and blood plasma, cerebrospinal fluid, cerumen and earwax, cowper's fluid or pre-ejaculatory fluid, chyle, chyme, stool, female ejaculate, interstitial fluid, intracellular fluid, lymph, menses, breast milk, mucus, pleural fluid, pus, saliva, sebum, semen, serum, sweat, synovial fluid, tears, urine, vaginal lubrication, vitreous humor, vomit). In some embodiments, the subject and/or control sample is selected from the group consisting of cells, cell lines, histological slides, paraffin embedded tissues, biopsies, whole blood, nipple aspirate, serum, plasma, buccal scrape, saliva, cerebrospinal fluid, urine, stool, and bone marrow. In some embodiments, the sample is serum, plasma, or urine. In other embodiments, the sample is serum.

The samples can be collected from individuals repeatedly over a longitudinal period of time (e.g., once or more on the order of days, weeks, months, annually, biannually, etc.). Obtaining numerous samples from an individual over a period of time can be used to verify results from earlier detections and/or to identify an alteration in biological pattern as a result of, for example, disease progression, drug treatment, etc. For example, subject samples can be taken and monitored every month, every two months, or combinations of one, two, or three month intervals according to the present invention. In addition, the cytokine/chemokine amount and/or activity measurements of the subject obtained over time can be conveniently compared with each other, as well as with those of normal controls during the monitoring period, thereby providing the subject's own values, as an internal, or personal, control for long-term monitoring.

Sample preparation and separation can involve any of the procedures, depending on the type of sample collected and/or analysis of biomarker measurement(s). Such procedures include, by way of example only, concentration, dilution, adjustment of pH, removal of high abundance polypeptides (e.g., albumin, gamma globulin, and transferrin, etc.), addition of preservatives and calibrants, addition of protease inhibitors, addition of denaturants, desalting of samples, concentration of sample proteins, extraction and purification of lipids.

### Pharmaceutical Compositions

The present invention provides pharmaceutically acceptable compositions of the compositions disclosed herein. As described in detail below, the pharmaceutical compositions of the present invention may be specially formulated for administration in solid or liquid form, including those adapted for the following: (1) oral administration, for example, drenches (aqueous or non-aqueous solutions or suspensions), tablets, boluses, powders, granules, pastes; (2) parenteral administration, for example, by subcutaneous, intramuscular or intravenous injection as, for example, a sterile solution or suspension; or (3) intrarectally, for example, as a pessary, cream or foam.

Compositions described herein may be used for oral administration to the gastrointestinal tract, directed at the objective of introducing the probiotic bacteria to tissues of the gastrointestinal tract. The formulation for a therapeutic composition of the present invention may also include other probiotic agents or nutrients which promote spore germination and/or bacterial growth. An exemplary material is a bifidogenic oligosaccharide, which promotes the growth of beneficial probiotic bacteria. In certain embodiment, the probiotic bacterial strain is combined with a therapeutically-effective dose of an (preferably, broad spectrum) antibiotic, or an anti-fungal agent. In some embodiments, the compositions described herein are encapsulated into an enterically-coated, time-released capsule or tablet. The enteric coating allows the capsule/tablet to remain intact (i.e., undisolved) as it passes through the gastrointestinal tract, until after a certain time and/or until it reaches a certain part of the GI tract (e.g., the small intestine). The time-released component prevents the “release” of the probiotic bacterial strain in the compositions described herein for a pre-determined time period.

The therapeutic compositions of the present invention may also include known antioxidants, buffering agents, and other agents such as coloring agents, flavorings, vitamins or minerals.

In some embodiments, the therapeutic compositions of the present invention are combined with a carrier which is physiologically compatible with the gastrointestinal tissue of the species to which it is administered. Carriers can be comprised of solid-based, dry materials for formulation into tablet, capsule or powdered form; or the carrier can be comprised of liquid or gel-based materials for formulations into liquid or gel forms. The specific type of carrier, as well as the final formulation depends, in part, upon the selected route(s) of administration. The therapeutic composition of the present invention may also include a variety of carriers and/or binders. A preferred carrier is micro-crystalline cellulose (MCC) added in an amount sufficient to complete the one gram dosage total weight. Carriers can be solid-based dry materials for formulations in tablet, capsule or powdered form, and can be liquid or gel-based materials for formulations in liquid or gel forms, which forms depend, in part, upon the routes of administration. Typical carriers for dry formulations include, but are not limited to: trehalose, malto-dextrin, rice flour, microcrystalline cellulose (MCC) magnesium stearate, inositol, FOS, GOS, dextrose, sucrose, and like carriers. Suitable liquid or gel-based carriers include but are not limited to: water and physiological salt solutions; urea; alcohols and derivatives (e.g., methanol, ethanol, propanol, butanol); glycols (e.g., ethylene glycol, propylene glycol, and the like). Preferably, water-based carriers possess a neutral pH value (i.e., pH 7.0). Other carriers or agents for administering the compositions described herein are known in the art, e.g., in U.S. Pat. No. 6,461,607.

The phrase “pharmaceutically acceptable” is employed herein to refer to those agents, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable carrier” as used herein means a pharmaceutically-acceptable material, composition or vehicle, such as a liquid or solid filler, diluent, excipient, solvent or encapsulating material, involved in carrying or transporting the subject chemical from one organ, or portion of the body, to another organ, or portion of the body. Each carrier must be “acceptable” in the sense of being compatible with the other ingredients of the formulation and not injurious to the subject. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

Formulations suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of one or more bacterial strains as disclosed herein.

In some embodiments, the composition (e.g., bacterial composition, pharmaceutical composition, nutraceutical composition) comprises at least one carbohydrate. A “carbohydrate” refers to a sugar or polymer of sugars. The terms “saccharide,” “polysaccharide,” “carbohydrate,” and “oligosaccharide” may be used interchangeably.

Most carbohydrates are aldehydes or ketones with many hydroxyl groups, usually one on each carbon atom of the molecule. Carbohydrates generally have the molecular formula CₙH₂ₙOₙ. A carbohydrate may be a monosaccharide, a disaccharide, trisaccharide, oligosaccharide, or polysaccharide. The most basic carbohydrate is a monosaccharide, such as glucose, sucrose, galactose, mannose, ribose, arabinose, xylose, and fructose. Disaccharides are two joined monosaccharides. Exemplary disaccharides include sucrose, maltose, cellobiose, and lactose. Typically, an oligosaccharide includes between three and six monosaccharide units (e.g., raffinose, stachyose), and polysaccharides include six or more monosaccharide units. Exemplary polysaccharides include starch, glycogen, and cellulose. Carbohydrates may contain modified saccharide units such as 2′-deoxyribose wherein a hydroxyl group is removed, 2′-fluororibose wherein a hydroxyl group is replaced with a fluorine, or N-acetylglucosamine, a nitrogen-containing form of glucose (e.g., 2′-fluororibose, deoxyribose, and hexose). Carbohydrates may exist in many different forms, for example, conformers, cyclic forms, acyclic forms, stereoisomers, tautomers, anomers, and isomers.

In some embodiments, the composition comprises at least one lipid. As used herein a “lipid” includes fats, oils, triglycerides, cholesterol, phospholipids, fatty acids in any form including free fatty acids. Fats, oils and fatty acids can be saturated, unsaturated (cis or trans) or partially unsaturated (cis or trans). In some embodiments the lipid comprises at least one fatty acid selected from lauric acid (12:0), myristic acid (14:0), palmitic acid (16:0), palmitoleic acid (16:1), margaric acid (17:0), heptadecenoic acid (17:1), stearic acid (18:0), oleic acid (18:1), linoleic acid (18:2), linolenic acid (18:3), octadecatetraenoic acid (18:4), arachidic acid (20:0), eicosenoic acid (20:1), eicosadienoic acid (20:2), eicosatetraenoic acid (20:4), eicosapentaenoic acid (20:5) (EPA), docosanoic acid (22:0), docosenoic acid (22:1), docosapentaenoic acid (22:5), docosahexaenoic acid (22:6) (DHA), and tetracosanoic acid (24:0). In some embodiments the composition comprises at least one modified lipid, for example a lipid that has been modified by cooking.

In some embodiments, the composition comprises at least one supplemental mineral or mineral source. Examples of minerals include, without limitation: chloride, sodium, calcium, iron, chromium, copper, iodine, zinc, magnesium, manganese, molybdenum, phosphorus, potassium, and selenium. Suitable forms of any of the foregoing minerals include soluble mineral salts, slightly soluble mineral salts, insoluble mineral salts, chelated minerals, mineral complexes, non-reactive minerals such as carbonyl minerals, and reduced minerals, and combinations thereof.

In some embodiments, the composition comprises at least one supplemental vitamin. The at least one vitamin can be fat-soluble or water soluble vitamins. Suitable vitamins include but are not limited to vitamin C, vitamin A, vitamin E, vitamin B12, vitamin K, riboflavin, niacin, vitamin D, vitamin B6, folic acid, pyridoxine, thiamine, pantothenic acid, and biotin. Suitable forms of any of the foregoing are salts of the vitamin, derivatives of the vitamin, compounds having the same or similar activity of the vitamin, and metabolites of the vitamin.

In some embodiments, the composition comprises an excipient. Non-limiting examples of suitable excipients include a buffering agent, a preservative, a stabilizer, a binder, a compaction agent, a lubricant, a dispersion enhancer, a disintegration agent, a flavoring agent, a sweetener, and a coloring agent.

In some embodiments, the excipient comprises a buffering agent. Non-limiting examples of suitable buffering agents include sodium citrate, magnesium carbonate, magnesium bicarbonate, calcium carbonate, and calcium bicarbonate.

In some embodiments, the excipient comprises a preservative. Non-limiting examples of suitable preservatives include antioxidants, such as alpha-tocopherol and ascorbate, and antimicrobials, such as parabens, chlorobutanol, and phenol.

In some embodiments, the composition comprises a binder as an excipient. Non-limiting examples of suitable binders include starches, pregelatinized starches, gelatin, polyvinylpyrolidone, cellulose, methylcellulose, sodium carboxymethylcellulose, ethylcellulose, polyacrylamides, polyvinyloxoazolidone, polyvinylalcohols, C₁₂-C₁₈ fatty acid alcohol, polyethylene glycol, polyols, saccharides, oligosaccharides, and combinations thereof.

In some embodiments, the composition comprises a lubricant as an excipient. Non-limiting examples of suitable lubricants include magnesium stearate, calcium stearate, zinc stearate, hydrogenated vegetable oils, sterotex, polyoxyethylene monostearate, talc, polyethyleneglycol, sodium benzoate, sodium lauryl sulfate, magnesium lauryl sulfate, and light mineral oil.

In some embodiments, the composition comprises a dispersion enhancer as an excipient. Non-limiting examples of suitable dispersants include starch, alginic acid, polyvinylpyrrolidones, guar gum, kaolin, bentonite, purified wood cellulose, sodium starch glycolate, isoamorphous silicate, and microcrystalline cellulose as high HLB emulsifier surfactants.

In some embodiments, the composition comprises a disintegrant as an excipient. In some embodiments the disintegrant is a non-effervescent disintegrant. Non-limiting examples of suitable non-effervescent disintegrants include starches such as corn starch, potato starch, pregelatinized and modified starches thereof, sweeteners, clays, such as bentonite, micro-crystalline cellulose, alginates, sodium starch glycolate, gums such as agar, guar, locust bean, karaya, pectin, and tragacanth. In some embodiments the disintegrant is an effervescent disintegrant. Non-limiting examples of suitable effervescent disintegrants include sodium bicarbonate in combination with citric acid, and sodium bicarbonate in combination with tartaric acid.

In some embodiments, the composition comprises maltodextrin (e.g., maltrin 100). In some embodiments, the composition comprises at least about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, or 35% maltodextrin. In some embodiments, the composition comprises no more than about 20%, 21%, 22%, 23%, 24%, 25%, 26%, 27%, 28%, 29%, 30%, 31%, 32%, 33%, 34%, 35%, 36%, 37%, 38%, 39%, or 40% maltodextrin. In some embodiments, the composition comprises at least about 15% but no more than about 35% maltodextrin. In some embodiments, the composition comprises at least about 15% but no more than about 25% maltodextrin. In some embodiments, the composition comprises about 20% maltodextrin. In some embodiments, the composition comprises at least about 50 mg, 55 mg, 60 mg, 65 mg, 70 mg, 75 mg, 80 mg, 85 mg, 90 mg, 95 mg, or 100 mg maltodextrin. In some embodiments, the composition comprises no more than about 120 mg, 125 mg, 130 mg, 135 mg, 140 mg, 145 mg, 150 mg, 155 mg, 160 mg, 165 mg, 170 mg, 175 mg, 180 mg, 185 mg, 190 mg, 195 mg, 200 mg, 205 mg, 210 mg, 215 mg, 220 mg, 225 mg, 230 mg, 235 mg, 240 mg, 245 mg, 250 mg, 255 mg, 260 mg, 265 mg, 270 mg, 275 mg, 280 mg, 285 mg, 290 mg, 295 mg, or 300 mg maltodextrin. In some embodiments, the composition comprises at least about 50 mg but no more than about 150 mg maltodextrin. In some embodiments, the composition comprises about 100 mg maltodextrin.

In some embodiments, the composition comprises magnesium stearate. In some embodiments, the composition comprises at least about 0.1%, 0.2%, 0.3%, 0.4%, 0.5%, 0.6%, 0.7%, 0.8%, 0.9%, or 1% magnesium stearate. In some embodiments, the composition comprises no more than about 1.5%, 1.6%, 1.7%, 1.8%, 1.9%, 2%, 2.1%, 2.2%, 2.3%, 2.4%, 2.5%, 2.6%, 2.7%, 2.8%, 2.9%, or 3% magnesium stearate. In some embodiments, the composition comprises at least about 0.5% but no more than about 1.5% magnesium stearate. In some embodiments, the composition comprises about 1% magnesium stearate.

In some embodiments, the composition comprises an anti-caking agent (e.g., silica, silicate). In some embodiments, the composition comprises Pirosil anti-caking agent, optionally selected from Pirosil PS-120, Pirosil PS-200, Pirosil PS-300, and Pirosil PS-2000. In some embodiments, the composition comprises Pirosil PS-200. In some embodiments, the composition comprises at least about 0.1%, 0.2%, 0.3%, 0.4%, 0.5%, 0.6%, 0.7%, 0.8%, 0.9%, or 1% anti-caking agent, optionally Pirosil PS-200. In some embodiments, the composition comprises no more than about 1.5%, 1.6%, 1.7%, 1.8%, 1.9%, 2%, 2.1%, 2.2%, 2.3%, 2.4%, 2.5%, 2.6%, 2.7%, 2.8%, 2.9%, or 3% anti-caking agent, optionally Pirosil PS-200. In some embodiments, the composition comprises at least about 0.5% but no more than about 1.5% anti-caking agent, optionally Pirosil PS-200. In some embodiments, the composition comprises about 1% anti-caking agent, optionally Pirosil PS-200.

In some embodiments, the composition comprises EMBO CAPS (World Wide Web at embocaps.com/). In some embodiments, the composition comprises EMBO CAPS VG #0.

In some embodiments, the composition comprises maltodextrin (e.g., maltrin 100), magnesium stearate, an anti-caking agent (e.g., Pirosil PS-200), EMBO CAPS VG #0, or any combination of two or more thereof.

In some embodiments, the composition is a food product (e.g., a food or beverage) such as a health food or beverage, a food or beverage for infants, a food or beverage for pregnant women, athletes, senior citizens or other specified group, a functional food, a beverage, a food or beverage for specified health use, a dietary supplement, a food or beverage for patients, or an animal feed. Specific examples of the foods and beverages include various beverages such as juices, refreshing beverages, tea beverages, drink preparations, jelly beverages, and functional beverages; alcoholic beverages such as beers; carbohydrate-containing foods such as rice food products, noodles, breads, and pastas; paste products such as fish hams, sausages, paste products of seafood; retort pouch products such as curries, food dressed with a thick starchy sauces, and Chinese soups; soups; dairy products such as milk, dairy beverages, ice creams, cheeses, and yogurts; fermented products such as fermented soybean pastes, yogurts, fermented beverages, and pickles; bean products; various confectionery products, including biscuits, cookies, and the like, candies, chewing gums, gummies, cold desserts including jellies, cream caramels, and frozen desserts; instant foods such as instant soups and instant soy-bean soups; microwavable foods; and the like. Further, the examples also include health foods and beverages prepared in the forms of powders, granules, tablets, capsules, liquids, pastes, and jellies.

### Nutraceutical Composition

A nuutraceutical composition is a pharmaceutical alternative which may have physiological benefits. In some embodiments, a nutraceutical composition is a food (or part of a food) that provides medical or health benefits, including the prevention and/or treatment of a disease. See, e.g., Brower (1998) Nat. Biotechnol. 16:728-731; Kalra (2003) AAPS Pharm Sci. 5 (3): 25. In other embodiments, a nutraceutical composition is a dietary or nutritional supplement.

Accordingly, a nutraceutical composition of the invention can be a food product, foodstuff, functional food, or a supplement composition for a food product or a foodstuff. As used herein, the term food product refers to any food or feed which provides a nutritional source and is suitable for oral consumption by humans or animals. The food product may be a prepared and packaged food (e.g., mayonnaise, salad dressing, bread, or cheese food) or an animal feed (e.g., extruded and pelleted animal feed, coarse mixed feed or pet food composition). As used herein, the term foodstuff refers to a nutritional source for human or animal oral consumption. Functional foods refer to foods being consumed as part of a usual diet but are demonstrated to have physiological benefits and/or reduce the risk of chronic disease beyond basic nutritional functions.

Food products, foodstuffs, functional foods, or dietary supplements may be beverages such as non-alcoholic and alcoholic drinks as well as liquid preparations to be added to drinking water and liquid food. Non-alcoholic drinks are for instance soft drinks; sport drinks; fruit juices, such as orange juice, apple juice and grapefruit juice; lemonades; teas; near-water drinks; and milk and other dairy drinks such as yogurt drinks, and diet drinks. In other embodiments, food products, foodstuffs, functional foods, or dietary supplements refer to solid or semi-solid foods. These forms can include, but are not limited to, baked goods such as cakes and cookies; puddings; dairy products; confections; snack foods (e.g., chips); or frozen confections or novelties (e.g., ice cream, milk shakes); prepared frozen meals; candy; liquid food such as soups; spreads; sauces; salad dressings; prepared meat products; cheese; yogurt and any other fat or oil containing foods; and food ingredients (e.g., wheat flour). In some embodiments, the food products, foodstuffs, functional foods, or dietary supplements may be in the form of tablets, boluses, powders, granules, pastes, pills or capsules for the ease of ingestion.

It is understood by those of skill in the art that in additional to isolated, and optionally purified and/or sonicated compositions of the present disclosure and other ingredients can be added to food products, foodstuffs, or functional foods described herein, for example, fillers, emulsifiers, preservatives, etc. for the processing or manufacture of the same. Additionally, flavors, coloring agents, spices, nuts and the like may be incorporated into the nutraceutical composition. Flavorings can be in the form of flavored extracts, volatile oils, chocolate flavorings, peanut butter flavoring, cookie crumbs, crisp rice, vanilla or any commercially available flavoring.

Emulsifiers can also be added for stability of the nutraceutical compositions. Examples of suitable emulsifiers include, but are not limited to, lecithin (e.g., from egg or soy), and/or mono- and di-glycerides. Other emulsifiers are readily apparent to the skilled artisan and selection of suitable emulsifier(s) will depend, in part, upon the formulation and final product. Preservatives can also be added to the nutritional supplement to extend product shelf life. Preferably, preservatives such as potassium sorbate, sodium sorbate, potassium benzoate, sodium benzoate or calcium disodium EDTA are used.

In addition, the nutraceutical composition can contain natural or artificial (preferably low calorie) sweeteners, e.g., saccharides, cyclamates, aspartamine, aspartame, acesulfame K, and/or sorbitol. Such artificial sweeteners can be desirable if the nutraceutical composition is intended to be consumed by an overweight or obese individual, or an individual with type II diabetes who is prone to hyperglycemia.

Moreover, a multi-vitamin and mineral supplement can be added to the nutraceutical compositions of the present invention to obtain an adequate amount of an essential nutrient, which is missing in some diets. The multi-vitamin and mineral supplement can also be useful for disease prevention and protection against nutritional losses and deficiencies due to lifestyle patterns.

As described herein, modulation of commensal bacterial populations can provide additional benefit against the development and progression of inflammatory diseases, autoimmune diseases, and cancer. Accordingly, particular embodiments of the invention provide for the nutritional source of the nutraceutical to modulate endogenous commensal bacterial populations. Such modulation can be achieved by modification of gut pH, consumption of beneficial bacteria (e.g., as in yogurt), by providing nutritional sources (e.g., prebiotics) that select for particular populations of bacteria, or by providing antibacterial compounds. Such modulation can mean an increase or decrease in the gut microbiota populations or ratios. In particular embodiments, the absolute or relative numbers of desirable gut microorganisms is increased and/or the absolute or relative numbers of undesirable gut microorganisms is decreased. For example, it is contemplated that there are a variety of nutritional sources exhibiting antibacterial activity that can be used to modulate gut microbiota populations. For example, garlic has been shown to produce the compound allicin (allyl 2-propenethiosulfinate), which exhibits antibacterial activity toward E. coli (Fujisawa, et al. (2009) Biosci. Biotechnol. Biochem. 73 (9): 1948-55; Fujisawa, et al. (2008) J. Agric. Food Chem. 56 (11): 4229-35). Similarly, rosemary extracts and other essential oils have been shown to contain antibacterial activity (Klancnik, et al. (2009) J. Food Prot. 72 (8): 1744-52; Si, et al. (2006) J. Appl. Microbiol. 100 (2): 296-305). Extracts of the edible basidiomycete, Lentinus edodes (Shiitake), have also been shown to possess antibiotic activity (Soboleva, et al. (2006) Antibiot. Khimioter. 51 (7): 3-8; Hirasawa, et al. (1999) Int. J. Antimicrob. Agents 11 (2): 151-7). Moreover, purple and red vegetable and fruit juices exhibit antibacterial activities (Lee, et al. (2003) Nutrition 19:994-996). Furthermore, it is contemplated herein that the food products, foodstuffs, functional foods, or dietary supplements may be combined with antibiotics to control the gut microbiota populations.

The nutraceutical composition of the present invention can be provided in a commercial package, alone, or with additional components, e.g., other food products, food stuffs, functional foods, dietary supplement. Desirably, the commercial package has instructions for consumption of the instant nutraceutical, including preparation and frequency of consumption, and use in the prevention or treatment of inflammatory diseases, autoimmune diseases and cancer. Moreover, in particular embodiments, the commercial package further includes a natural product (e.g., the food, extracts, antibiotics, and oils) that modulates endogenous commensal bacterial populations. A package containing both a nutraceutical of the present disclosure in combination with said natural product can contain instructions for consuming the natural product, e.g., in advance (e.g., 2, 4, 6 or 8 or more hours) of consuming the nutraceutical in order to enhance the activity of the nutraceutical composition.

### Subjects

In certain embodiments, the subject suitable for the compositions and methods disclosed herein is a mammal (e.g., mouse, rat, primate, non-human mammal, domestic animal, such as a dog, cat, cow, horse, and the like), and is preferably a human. In some embodiments, the subject is healthy. In some embodiments, the subject is afflicted with a disease (e.g., a cancer or an inflammatory/autoimmune disease).

In other embodiments, the subject is an animal model of a cancer. For example, the animal model can be an orthotopic xenograft animal model of human oral squamous carcinoma, or comprising cancer stem cells (CSCs)/undifferentiated tumors. In some embodiments, the subject is an animal model of an inflammatory disease or an autoimmune disease.

In some embodiments, the subject has not undergone treatment, such as chemotherapy, radiation therapy, targeted therapy, and/or anti-immune therapy (such as NK cell-related immunotherapies). In still other embodiments, the subject has undergone treatment, such as chemotherapy, radiation therapy, targeted therapy, and/or anti-immune therapy (such as NK cell-related immunotherapies).

In certain embodiments, the subject has had surgery to remove cancerous or pre-cancerous tissue. In other embodiments, the cancerous tissue has not been removed, e.g., the cancerous tissue may be located in an inoperable region of the body, such as in a tissue that is essential for life, or in a region where a surgical procedure would cause considerable risk of harm to the patient.

In certain embodiments, the subject is in need of an NK cell activation. In certain embodiments, the subject is in need of an increased level of certain cytokines (e.g., cytokines or chemokines described herein). In certain embodiments, the subject would benefit from the compositions or methods of the present disclosure, irrespective of whether they would need e.g., an NK cell activation.

The methods of the present invention can be used to treat and/or determine the responsiveness to a composition comprising at least one of probiotic bacteria, alone or in combination with other NK immunotherapies, of many different cancers in subjects such as those described herein.

### Amyotrophic Lateral Sclerosis (ALS) Therapies

Two drugs, riluzole and edaravone, are currently available to delay the progression of the disease. Riluzole prolongs ALS survival; it increases survival rates at 12 months by 10% and prolongs survival by 6 months. Similarly Edaravone is effective in treating ALS.

Masitinib is a tyrosine kinase inhibitor used to treat cancer in dogs. It was proven that mastinib inhibited glial cell activation in the appropriate rat model and increased survival.

Retigabine is an approved drug for epilepsy, and acts by binding to the voltage-gated potassium channels and increasing the M-current, thus leading to membrane hyperpolarization. Retigabine is able to prolong motor neuron survival and decrease excitability, which is advantageous in the treatment of ALS, since it is believed that, in this disease, neurons are hyper-excitable, firing more than normal and ultimately leading to cell death. This drug is still under clinical trial for the treatment of ALS.

Tamoxifen is an antioestrogen drug, approved for the chemotherapy and chemoprevention of breast cancer. The repurposing of this drug for the treatment of ALS arose serendipitously, after the observation of a neurological improvement in patients and disease stabilization in ALS patients with breast cancer treated with tamoxifen. Its neuroprotective properties appear to be related to inhibition of protein kinase C, which is overexpressed in the spinal cord of ALS patients. Moreover, tamoxifen was found to be able to modulate a proteinopathy present in ALS, through its capacity to be an autophagy modulator.

### Cancer Therapies

The therapeutic agents of the present invention can be used alone or can be administered in combination therapy with, e.g., chemotherapeutic agents, hormones, antiangiogens, radiolabelled, compounds, or with surgery, cryotherapy, and/or radiotherapy. The preceding treatment methods can be administered in conjunction with other forms of conventional therapy (e.g., standard-of-care treatments for cancer well-known to the skilled artisan), either consecutively with, pre- or post-conventional therapy. For example, agents of the present invention can be administered with a therapeutically effective dose of chemotherapeutic agent. In other embodiments, agents of the present invention are administered in conjunction with chemotherapy to enhance the activity and efficacy of the chemotherapeutic agent. The Physicians' Desk Reference (PDR) discloses dosages of chemotherapeutic agents that have been used in the treatment of various cancers. The dosing regimen and dosages of these aforementioned chemotherapeutic drugs that are therapeutically effective will depend on the particular cancer being treated, the extent of the disease and other factors familiar to the physician of skill in the art, and can be determined by the physician.

Immunotherapy is a targeted therapy that may comprise, for example, the use of cancer vaccines and/or sensitized antigen presenting cells. For example, an oncolytic virus is a virus that is able to infect and lyse cancer cells, while leaving normal cells unharmed, making them potentially useful in cancer therapy. Replication of oncolytic viruses both facilitates tumor cell destruction and also produces dose amplification at the tumor site. They may also act as vectors for anticancer genes, allowing them to be specifically delivered to the tumor site. The immunotherapy can involve passive immunity for short-term protection of a host, achieved by the administration of pre-formed antibody directed against a cancer antigen or disease antigen (e.g., administration of a monoclonal antibody, optionally linked to a chemotherapeutic agent or toxin, to a tumor antigen). For example, anti-VEGF is known to be effective in treating renal cell carcinoma. Immunotherapy can also focus on using the cytotoxic lymphocyte-recognized epitopes of cancer cell lines. Alternatively, antisense polynucleotides, ribozymes, RNA interference molecules, triple helix polynucleotides and the like, can be used to selectively modulate biomolecules that are linked to the initiation, progression, and/or pathology of a tumor or cancer.

Immunotherapy also encompasses immune checkpoint modulators. Immune checkpoints are a group of molecules on the cell surface of CD4+ and/or CD8+ T cells that fine-tune immune responses by down-modulating or inhibiting an anti-tumor immune response. Immune checkpoint proteins are well-known in the art and include, without limitation, CTLA-4, PD-1, VISTA, B7-H2, B7-H3, PD-L1, B7-H4, B7-H6, 2B4, ICOS, HVEM, PD-L2, CD160, gp49B, PIR-B, KIR family receptors, TIM-1, TIM-3, TIM-4, LAG-3, BTLA, SIRPalpha (CD47), CD48, 2B4 (CD244), B7.1, B7.2, ILT-2, ILT-4, TIGIT, HHLA2, TMIDG2, KIR3DL3, and A2aR (see, for example, WO 2012/177624). Inhibition of one or more immune checkpoint inhibitors can block or otherwise neutralize inhibitory signaling to thereby upregulate an immune response in order to more efficaciously treat cancer. In some embodiments, the composition of the present disclosure is administered in combination with one or more inhibitors of immune checkpoints, such as PD1, PD-L1, and/or CD47 inhibitors.

Adoptive cell-based immunotherapies can be combined with the therapies of the present invention. Well-known adoptive cell-based immunotherapeutic modalities, including, without limitation, irradiated autologous or allogeneic tumor cells, tumor lysates or apoptotic tumor cells, antigen-presenting cell-based immunotherapy, dendritic cell-based immunotherapy, adoptive T cell transfer, adoptive CAR T cell therapy, autologous immune enhancement therapy (AIET), cancer vaccines, and/or antigen presenting cells. Such cell-based immunotherapies can be further modified to express one or more gene products to further modulate immune responses, such as expressing cytokines like GM-CSF, and/or to express tumor-associated antigen (TAA) antigens, such as Mage-1, gp-100, and the like.

In other embodiments, immunotherapy comprises non-cell-based immunotherapies. In some embodiments, compositions comprising antigens with or without vaccine-enhancing adjuvants are used. Such compositions exist in many well-known forms, such as peptide compositions, oncolytic viruses, recombinant antigen comprising fusion proteins, and the like. In some embodiments, immunomodulatory cytokines, such as interferons, G-CSF, imiquimod, TNFalpha, and the like, as well as modulators thereof (e.g., blocking antibodies or more potent or longer lasting forms) are used. In some embodiments, immunomodulatory interleukins, such as IL-2, IL-6, IL-7, IL-12, IL-17, IL-23, and the like, as well as modulators thereof (e.g., blocking antibodies or more potent or longer lasting forms) are used. In some embodiments, immunomodulatory chemokines, such as CCL3, CCL26, and CXCL7, and the like, as well as modulators thereof (e.g., blocking antibodies or more potent or longer lasting forms) are used. In some embodiments, immunomodulatory molecules targeting immunosuppression, such as STAT3 signaling modulators, NFkappaB signaling modulators, and immune checkpoint modulators, are used. The terms “immune checkpoint” and “anti-immune checkpoint therapy” are described above.

In still other embodiments, immunomodulatory drugs, such as immunocytostatic drugs, glucocorticoids, cytostatics, immunophilins and modulators thereof (e.g., rapamycin, a calcineurin inhibitor, tacrolimus, ciclosporin (cyclosporin), pimecrolimus, abetimus, gusperimus, ridaforolimus, everolimus, temsirolimus, zotarolimus, etc.), hydrocortisone (cortisol), cortisone acetate, prednisone, prednisolone, methylprednisolone, dexamethasone, betamethasone, triamcinolone, beclometasone, fludrocortisone acetate, deoxycorticosterone acetate (doca) aldosterone, a non-glucocorticoid steroid, a pyrimidine synthesis inhibitor, leflunomide, teriflunomide, a folic acid analog, methotrexate, anti-thymocyte globulin, anti-lymphocyte globulin, thalidomide, lenalidomide, pentoxifylline, bupropion, curcumin, catechin, an opioid, an IMPDH inhibitor, mycophenolic acid, myriocin, fingolimod, an NF-xB inhibitor, raloxifene, drotrecogin alfa, denosumab, an NF-xB signaling cascade inhibitor, disulfiram, olmesartan, dithiocarbamate, a proteasome inhibitor, bortezomib, MG132, Prol, NPI-0052, curcumin, genistein, resveratrol, parthenolide, thalidomide, lenalidomide, flavopiridol, non-steroidal anti-inflammatory drugs (NSAIDs), arsenic trioxide, dehydroxymethylepoxyquinomycin (DHMEQ), 13C (indole-3-carbinol)/DIM (di-indolmethane) (13C/DIM), Bay 11-7082, luteolin, cell permeable peptide SN-50, IKBa.-super repressor overexpression, NFKB decoy oligodeoxynucleotide (ODN), or a derivative or analog of any thereo, are used. In yet other embodiments, immunomodulatory antibodies or protein are used. For example, antibodies that bind to CD40, Toll-like receptor (TLR), OX40, GITR, CD27, or to 4-1BB, T-cell bispecific antibodies, an anti-IL-2 receptor antibody, an anti-CD3 antibody, OKT3 (muromonab), otelixizumab, teplizumab, visilizumab, an anti-CD4 antibody, clenoliximab, keliximab, zanolimumab, an anti-CD11 a antibody, efalizumab, an anti-CD18 antibody, erlizumab, rovelizumab, an anti-CD20 antibody, afutuzumab, ocrelizumab, ofatumumab, pascolizumab, rituximab, an anti-CD23 antibody, lumiliximab, an anti-CD40 antibody, teneliximab, toralizumab, an anti-CD40L antibody, ruplizumab, an anti-CD62L antibody, aselizumab, an anti-CD80 antibody, galiximab, an anti-CD147 antibody, gavilimomab, a B-Lymphocyte stimulator (BLyS) inhibiting antibody, belimumab, an CTLA4-Ig fusion protein, abatacept, belatacept, an anti-CTLA4 antibody, ipilimumab, tremelimumab, an anti-eotaxin 1 antibody, bertilimumab, an anti-a4-integrin antibody, natalizumab, an anti-IL-6R antibody, tocilizumab, an anti-LFA-1 antibody, odulimomab, an anti-CD25 antibody, basiliximab, daclizumab, inolimomab, an anti-CD5 antibody, zolimomab, an anti-CD2 antibody, siplizumab, nerelimomab, faralimomab, atlizumab, atorolimumab, cedelizumab, dorlimomab aritox, dorlixizumab, fontolizumab, gantenerumab, gomiliximab, lebrilizumab, maslimomab, morolimumab, pexelizumab, reslizumab, rovelizumab, talizumab, telimomab aritox, vapaliximab, vepalimomab, aflibercept, alefacept, rilonacept, an IL-1 receptor antagonist, anakinra, an anti-IL-5 antibody, mepolizumab, an IgE inhibitor, omalizumab, talizumab, an IL12 inhibitor, an IL23 inhibitor, ustekinumab, and the like.

Nutritional supplements that enhance immune responses, such as vitamin A, vitamin E, vitamin C, and the like, are well-known in the art (see, for example, U.S. Pat. Nos. 4,981,844 and 5,230,902 and PCT Publ. No. WO 2004/004483) can be used in the methods described herein.

Similarly, agents and therapies other than immunotherapy or in combination thereof can be used with in combination with an anti-KHK antibodies to treat a condition that would benefit therefrom. For example, chemotherapy, radiation, epigenetic modifiers (e.g., histone deacetylase (HDAC) modifiers, methylation modifiers, phosphorylation modifiers, and the like), targeted therapy, and the like are well-known in the art.

In some embodiments, chemotherapy is used. Chemotherapy includes the administration of a chemotherapeutic agent. Such a chemotherapeutic agent may be, but is not limited to, those selected from among the following groups of compounds: platinum compounds, cytotoxic antibiotics, antimetabolites, anti-mitotic agents, alkylating agents, arsenic compounds, DNA topoisomerase inhibitors, taxanes, nucleoside analogues, plant alkaloids, and toxins; and synthetic derivatives thereof. Exemplary compounds include, but are not limited to, alkylating agents: cisplatin, treosulfan, and trofosfamide; plant alkaloids: vinblastine, paclitaxel, docetaxol; DNA topoisomerase inhibitors: teniposide, crisnatol, and mitomycin; anti-folates: methotrexate, mycophenolic acid, and hydroxyurea; pyrimidine analogs: 5-fluorouracil, doxifluridine, and cytosine arabinoside; purine analogs: mercaptopurine and thioguanine; DNA antimetabolites: 2′-deoxy-5-fluorouridine, aphidicolin glycinate, and pyrazoloimidazole; and antimitotic agents: halichondrin, colchicine, and rhizoxin. Compositions comprising one or more chemotherapeutic agents (e.g., FLAG, CHOP) may also be used. FLAG comprises fludarabine, cytosine arabinoside (Ara-C) and G-CSF. CHOP comprises cyclophosphamide, vincristine, doxorubicin, and prednisone. In another embodiments, PARP (e.g., PARP-1 and/or PARP-2) inhibitors are used and such inhibitors are well-known in the art (e.g., Olaparib, ABT-888, BSI-201, BGP-15 (N-Gene Research Laboratories, Inc.); INO-1001 (Inotek Pharmaceuticals Inc.); PJ34 (Soriano et al., 2001; Pacher et al., 2002b); 3-aminobenzamide (Trevigen); 4-amino-1,8-naphthalimide; (Trevigen); 6 (5H)-phenanthridinone (Trevigen); benzamide (U.S. Pat. Re. 36,397); and NU1025 (Bowman et al.). The mechanism of action is generally related to the ability of PARP inhibitors to bind PARP and decrease its activity. PARP catalyzes the conversion of .beta.-nicotinamide adenine dinucleotide (NAD+) into nicotinamide and poly-ADP-ribose (PAR). Both poly (ADP-ribose) and PARP have been linked to regulation of transcription, cell proliferation, genomic stability, and carcinogenesis (Bouchard V. J. et.al. Experimental Hematology, Volume 31, Number 6, June 2003, pp. 446-454 (9); Herceg Z.; Wang Z.-Q. Mutation Research/Fundamental and Molecular Mechanisms of Mutagenesis, Volume 477, Number 1, 2 Jun. 2001, pp. 97-110 (14)). Poly (ADP-ribose) polymerase 1 (PARP1) is a key molecule in the repair of DNA single-strand breaks (SSBs) (de Murcia J. et al. 1997. Proc Natl Acad Sci USA 94:7303-7307; Schreiber V, Dantzer F, Ame J C, de Murcia G (2006) Nat Rev Mol Cell Biol 7:517-528; Wang Z Q, et al. (1997) Genes Dev 11:2347-2358). Knockout of SSB repair by inhibition of PARP1 function induces DNA double-strand breaks (DSBs) that can trigger synthetic lethality in cancer cells with defective homology-directed DSB repair (Bryant H E, et al. (2005) Nature 434:913-917; Farmer H, et al. (2005) Nature 434:917-921). The foregoing examples of chemotherapeutic agents are illustrative, and are not intended to be limiting.

In other embodiments, radiation therapy is used. The radiation used in radiation therapy can be ionizing radiation. Radiation therapy can also be gamma rays, X-rays, or proton beams. Examples of radiation therapy include, but are not limited to, external-beam radiation therapy, interstitial implantation of radioisotopes (I-125, palladium, iridium), radioisotopes such as strontium-89, thoracic radiation therapy, intraperitoneal P-32 radiation therapy, and/or total abdominal and pelvic radiation therapy. For a general overview of radiation therapy, see Hellman, Chapter 16: Principles of Cancer Management: Radiation Therapy, 6th edition, 2001, DeVita et al., eds., J. B. Lippencott Company, Philadelphia. The radiation therapy can be administered as external beam radiation or teletherapy wherein the radiation is directed from a remote source. The radiation treatment can also be administered as internal therapy or brachytherapy wherein a radioactive source is placed inside the body close to cancer cells or a tumor mass. Also encompassed is the use of photodynamic therapy comprising the administration of photosensitizers, such as hematoporphyrin and its derivatives, Vertoporfin (BPD-MA), phthalocyanine, photosensitizer Pc4, demethoxy-hypocrellin A; and 2BA-2-DMHA.

In other embodiments, hormone therapy is used. Hormonal therapeutic treatments can comprise, for example, hormonal agonists, hormonal antagonists (e.g., flutamide, bicalutamide, tamoxifen, raloxifene, leuprolide acetate (LUPRON), LH-RH antagonists), inhibitors of hormone biosynthesis and processing, and steroids (e.g., dexamethasone, retinoids, deltoids, betamethasone, cortisol, cortisone, prednisone, dehydrotestosterone, glucocorticoids, mineralocorticoids, estrogen, testosterone, progestins), vitamin A derivatives (e.g., all-trans retinoic acid (ATRA)); vitamin D3 analogs; antigestagens (e.g., mifepristone, onapristone), or antiandrogens (e.g., cyproterone acetate).

In other embodiments, photodynamic therapy (also called PDT, photoradiation therapy, phototherapy, or photochemotherapy) is used for the treatment of some types of cancer. It is based on the discovery that certain chemicals known as photosensitizing agents can kill one-celled organisms when the organisms are exposed to a particular type of light.

In yet other embodiments, laser therapy is used to harness high-intensity light to destroy cancer cells. This technique is often used to relieve symptoms of cancer such as bleeding or obstruction, especially when the cancer cannot be cured by other treatments. It may also be used to treat cancer by shrinking or destroying tumors.

The immunotherapy and/or cancer therapy may be administered before, after, or concurrently with the compositions described herein. The duration and/or dose of treatment with the compositions may vary according to the particular composition, or the particular combinatory therapy. An appropriate treatment time for a particular cancer therapeutic agent will be appreciated by the skilled artisan. The invention contemplates the continued assessment of optimal treatment schedules for each cancer therapeutic agent, where the phenotype of the cancer of the subject as determined by the methods of the invention is a factor in determining optimal treatment doses and schedules.

### Clinical Efficacy

Clinical efficacy can be measured by any method known in the art. For example, the response to a therapy (e.g., compositions of the present disclosure), relates to e.g., any response of the cancer, e.g., a tumor, to the therapy, preferably to a change in tumor mass and/or volume after initiation of neoadjuvant or adjuvant chemotherapy. Tumor response may be assessed in a neoadjuvant or adjuvant situation where the size of a tumor after systemic intervention can be compared to the initial size and dimensions as measured by CT, PET, mammogram, ultrasound or palpation and the cellularity of a tumor can be estimated histologically and compared to the cellularity of a tumor biopsy taken before initiation of treatment. Response may also be assessed by caliper measurement or pathological examination of the tumor after biopsy or surgical resection. Response may be recorded in a quantitative fashion like percentage change in tumor volume or cellularity or using a semi-quantitative scoring system such as residual cancer burden (Symmans et al. (2007) J. Clin. Oncol. 25:4414-4422) or Miller-Payne score (Ogston et al. (2003) Breast (Edinburgh, Scotland) 12:320-327) in a qualitative fashion like “pathological complete response” (pCR), “clinical complete remission” (cCR), “clinical partial remission” (cPR), “clinical stable disease” (cSD), “clinical progressive disease” (cPD) or other qualitative criteria. Assessment of tumor response may be performed early after the onset of neoadjuvant or adjuvant therapy, e.g., after a few hours, days, weeks or preferably after a few months. A typical endpoint for response assessment is upon termination of neoadjuvant chemotherapy or upon surgical removal of residual tumor cells and/or the tumor bed.

In some embodiments, clinical efficacy of the therapeutic treatments described herein may be determined by measuring the clinical benefit rate (CBR). The clinical benefit rate is measured by determining the sum of the percentage of patients who are in complete remission (CR), the number of patients who are in partial remission (PR) and the number of patients having stable disease (SD) at a time point at least 6 months out from the end of therapy. The shorthand for this formula is CBR=CR+PR+SD over 6 months. In some embodiments, the CBR for a particular regimen is at least 25%, 30%, 35%, 40%, 45%, 50%, 55%, 60%, 65%, 70%, 75%, 80%, 85%, or more.

Additional criteria for evaluating the response to a therapy (e.g., a composition of the present disclosure) are related to “survival,” which includes all of the following: survival until mortality, also known as overall survival (wherein said mortality may be either irrespective of cause or tumor related); “recurrence-free survival” (wherein the term recurrence shall include both localized and distant recurrence); metastasis free survival; disease free survival (wherein the term disease shall include cancer and diseases associated therewith). The length of said survival may be calculated by reference to a defined start point (e.g., time of diagnosis or start of treatment) and end point (e.g., death, recurrence or metastasis). In addition, criteria for efficacy of treatment can be expanded to include response to chemotherapy, probability of survival, probability of metastasis within a given time period, and probability of tumor recurrence.

For example, in order to determine appropriate threshold values, a particular agent encompassed by the present invention can be administered to a population of subjects and the outcome can be correlated to biomarker measurements that were determined prior to administration of a therapy (e.g., a composition of the present disclosure). The outcome measurement may be pathologic response to therapy given in the neoadjuvant setting. Alternatively, outcome measures, such as overall survival and disease-free survival can be monitored over a period of time for subjects following a therapy (e.g., a composition of the present disclosure). In certain embodiments, the same doses of the agent are administered to each subject. In related embodiments, the doses administered are standard doses known in the art for the agent. The period of time for which subjects are monitored can vary. For example, subjects may be monitored for at least 2, 4, 6, 8, 10, 12, 14, 16, 18, 20, 25, 30, 35, 40, 45, 50, 55, or 60 months.

### Exemplary Diseases

### Inflammatory Disorders

The compositions and methods described herein can be used, for example, for preventing or treating (reducing, partially or completely, the adverse effects of) an inflammatory disease or an autoimmune disease, such as chronic inflammatory bowel disease, systemic lupus erythematosus, psoriasis, muckle-wells syndrome, rheumatoid arthritis, multiple sclerosis, or Hashimoto's disease; an allergic disease, such as a food allergy, pollenosis, or asthma; an infectious disease, e.g., infection with Clostridium difficile; an inflammatory disease such as a TNF-mediated inflammatory disease (e.g., an inflammatory disease of the gastrointestinal tract, such as pouchitis, a cardiovascular inflammatory condition, such as atherosclerosis, or an inflammatory lung disease, such as chronic obstructive pulmonary disease); a pharmaceutical composition for suppressing rejection in organ transplantation or other situations in which tissue rejection might occur; a pharmaceutical composition for improving immune functions; or a pharmaceutical composition for suppressing the proliferation or function of immune cells.

In some embodiments, the methods and compositions provided herein are useful for the treatment or prevention of inflammation. In certain embodiments, the inflammation of any tissue and organs of the body, including musculoskeletal inflammation, vascular inflammation, neural inflammation, digestive system inflammation, ocular inflammation, inflammation of the reproductive system, and other inflammation, as discussed below.

Inflammatory or autoimmune diseases of the musculoskeletal system include, but are not limited, to those conditions affecting skeletal joints, including joints of the hand, wrist, elbow, shoulder, jaw, spine, neck, hip, knew, ankle, and foot, and conditions affecting tissues connecting muscles to bones such as tendons. Examples of such inflammatory or autoimmune diseases, which may be treated with the methods and compositions described herein include, but are not limited to, arthritis (including, for example, osteoarthritis, rheumatoid arthritis, psoriatic arthritis, ankylosing spondylitis, acute and chronic infectious arthritis, arthritis associated with gout and pseudogout, and juvenile idiopathic arthritis), tendonitis, synovitis, tenosynovitis, bursitis, fibrositis (fibromyalgia), epicondylitis, myositis, and osteitis (including, for example, Paget's disease, osteitis pubis, and osteitis fibrosa cystic).

Ocular inflammatory or autoimmune diseases refers to an inflammatory or autoimmune disease that affects any structure of the eye, including the eye lids. Examples of ocular inflammatory or autoimmune diseases which may be treated with the methods and compositions described herein include, but are not limited to, blepharitis, blepharochalasis, conjunctivitis, dacryoadenitis, keratitis, keratoconjunctivitis sicca (dry eye), scleritis, trichiasis, and uveitis

Examples of nervous system inflammatory or autoimmune diseases which may be treated with the methods and compositions described herein include, but are not limited to, encephalitis, Guillain-Barre syndrome, meningitis, neuromyotonia, narcolepsy, multiple sclerosis, myelitis and schizophrenia. Examples of inflammation of the vasculature or lymphatic system which may be treated with the methods and compositions described herein include, but are not limited to, arthrosclerosis, arthritis, phlebitis, vasculitis, and lymphangitis.

Examples of digestive system inflammatory or autoimmune diseases which may be treated with the methods and pharmaceutical compositions described herein include, but are not limited to, cholangitis, cholecystitis, enteritis, enterocolitis, gastritis, gastroenteritis, inflammatory bowel disease, ileitis, and proctitis. Inflammatory bowel diseases include, for example, certain art-recognized forms of a group of related conditions. Several major forms of inflammatory bowel diseases are known, with Crohn's disease (regional bowel disease, e.g., inactive and active forms) and ulcerative colitis (e.g., inactive and active forms) the most common of these diseases. In addition, the inflammatory bowel disease encompasses irritable bowel syndrome, microscopic colitis, lymphocytic-plasmocytic enteritis, coeliac disease, collagenous colitis, lymphocytic colitis and eosinophilic enterocolitis. Other less common forms of IBD include indeterminate colitis, pseudomembranous colitis (necrotizing colitis), ischemic inflammatory bowel disease, Behcet's disease, sarcoidosis, scleroderma, IBD-associated dysplasia, dysplasia associated masses or lesions, and primary sclerosing cholangitis.

Examples of reproductive system inflammatory or autoimmune diseases which may be treated with the methods and pharmaceutical compositions described herein include, but are not limited to, cervicitis, chorioamnionitis, endometritis, epididymitis, omphalitis, oophoritis, orchitis, salpingitis, tubo-ovarian abscess, urethritis, vaginitis, vulvitis, and vulvodynia.

The compositions and methods described herein may be used to prevent or treat autoimmune conditions having an inflammatory component. Such conditions include, but are not limited to, acute disseminated alopecia universalise, Behcet's disease, Chagas' disease, chronic fatigue syndrome, dysautonomia, encephalomyelitis, ankylosing spondylitis, aplastic anemia, hidradenitis suppurativa, autoimmune hepatitis, autoimmune oophoritis, celiac disease, Crohn's disease, diabetes mellitus type 1, type 2 diabetes, giant cell arteritis, goodpasture's syndrome, Grave's disease, Guillain-Barre syndrome, Hashimoto's disease, Henoch-Schonlein purpura, Kawasaki's disease, lupus erythematosus, microscopic colitis, microscopic polyarteritis, mixed connective tissue disease, Muckle-Wells syndrome, multiple sclerosis, myasthenia gravis, opsoclonus myoclonus syndrome, optic neuritis, ord's thyroiditis, pemphigus, polyarteritis nodosa, polymyalgia, rheumatoid arthritis, Reiter's syndrome, Sjogren's syndrome, temporal arteritis, Wegener's granulomatosis, warm autoimmune haemolytic anemia, interstitial cystitis, Lyme disease, morphea, psoriasis, sarcoidosis, scleroderma, ulcerative colitis, and vitiligo.

The compositions and methods described herein may be used to prevent or treat T-cell mediated hypersensitivity diseases having an inflammatory component. Such conditions include, but are not limited to, contact hypersensitivity, contact dermatitis (including that due to poison ivy), uticaria, skin allergies, respiratory allergies (hay fever, allergic rhinitis, house dustmite allergy) and gluten-sensitive enteropathy (Celiac disease).

Other inflammatory or autoimmune diseases which may be treated with the methods and pharmaceutical compositions include, for example, appendicitis, dermatitis, dermatomyositis, endocarditis, fibrositis, gingivitis, glossitis, hepatitis, hidradenitis suppurativa, iritis, laryngitis, mastitis, myocarditis, nephritis, otitis, pancreatitis, parotitis, percarditis, peritonoitis, pharyngitis, pleuritis, pneumonitis, prostatistis, pyelonephritis, and stomatisi, transplant rejection (involving organs such as kidney, liver, heart, lung, pancreas (e.g., islet cells), bone marrow, cornea, small bowel, skin allografts, skin homografts, and heart valve xengrafts, sewrum sickness, and graft vs host disease), acute pancreatitis, chronic pancreatitis, acute respiratory distress syndrome, Sexary's syndrome, congenital adrenal hyperplasis, nonsuppurative thyroiditis, hypercalcemia associated with cancer, pemphigus, bullous dermatitis herpetiformis, severe erythema multiforme, exfoliative dermatitis, seborrheic dermatitis, seasonal or perennial allergic rhinitis, bronchial asthma, contact dermatitis, atopic dermatitis, drug hypersensistivity reactions, allergic conjunctivitis, keratitis, herpes zoster ophthalmicus, iritis and oiridocyclitis, chorioretinitis, optic neuritis, symptomatic sarcoidosis, fulminating or disseminated pulmonary tuberculosis chemotherapy, idiopathic thrombocytopenia purpura in adults, secondary thrombocytopenia in adults, acquired (autoimmune) haemolytic anemia, regional enteritis, autoimmune vasculitis, multiple sclerosis, chronic obstructive pulmonary disease, solid organ transplant rejection, sepsis. Preferred treatments include treatment of transplant rejection, rheumatoid arthritis, psoriatic arthritis, multiple sclerosis, Type 1 diabetes, asthma, inflammatory bowel disease, systemic lupus erythematosus, psoriasis, chronic obstructive pulmonary disease, and inflammation accompanying infectious conditions (e.g., sepsis).

### Amyotrophic Lateral Sclerosis (ALS)

Amyotrophic lateral sclerosis (Lou Gehrig's Disease; ALS), also known as motor neurone disease, is considered the most common form of a motoneuron disease with an onset in adult age of, in average, about 50-60 years and an incidence of 1:50,000 per year.

ALS results in the death of neurons controlling voluntary muscles. ALS is characterized by stiff muscles, muscle twitching, and gradually worsening weakness due to muscles decreasing in size. It may begin with weakness in the arms or legs, or with difficulty speaking or swallowing. About half of the people affected develop at least mild difficulties with thinking and behavior and most people experience pain. Most eventually lose the ability to walk, use their hands, speak, swallow, and breathe. ALS is a progressive disease with a fatal outcome due to gradual paralysis of all voluntary muscles throughout the body, whereby the breathing and swallowing muscles become affected early on already.

The cause is not known in 90% to 95% of cases, but is believed to involve both genetic and environmental factors. The remaining 5-10% of cases are inherited from a person's parents. The most common familial forms of ALS in adults are caused by mutations of the superoxide dismutase gene, or SOD1, located on chromosome 21. The underlying mechanism involves damage to both upper and lower motor neurons.

No cure for ALS is known. The goal of current treatment is to improve symptoms. A medication called riluzole may extend life by about two to three months. Non-invasive ventilation may result in both improved quality and length of life. Mechanical ventilation can prolong survival but does not stop disease progression. A feeding tube may help. The disease can affect people of any age, but usually starts around the age of 60 and in inherited cases around the age of 50. The average survival from onset to death is two to four years, though this can vary. About 10% survive longer than 10 years. Most die from respiratory failure.

### Neuroinflammatory Diseases

The compositions and methods described herein may be used to prevent or treat inflammatory diseases including neuroinflammatory diseases. In certain embodiments, the neuroinflammatory diseases is Parkinson's disease, Alzheimer's disease, prion disease, Huntington's disease, motor neuron diseases (MND), spinocerebellar ataxia, spinal muscular atrophy, dystonia, idiopathicintracranial hypertension, epilepsy, nervous system disease, central nervous system disease, movement diseases, multiple sclerosis, encephalopathy, peripheral neuropathy, post-operative cognitive dysfunction, frontotemporal dementia, stroke, transient ischemic attack, vascular dementia, Creutzfeldt-Jakob disease, multiple sclerosis, prion disease, Pick's disease, corticobasal degeneration, Parkinson's disease, Lewy body dementia, progressive supranuclear palsy, dementia pugilistica (chronic traumatic encephalopathy), frontotemporal dementia, parkinsonism linked to chromosome 17, Lytico-Bodig disease, Tangle-predominant dementia, ganglioglioma, gangliocytoma, meningioangiomatosis, subacute sclerosing panencephalitis, lead encephalopathy, tuberous sclerosis, Hallervorden-Spatz disease, lipofuscinosis, argyrophilic grain disease, and frontotemporal lobar degeneration.

Furthermore, neuroinflammatory diseases include, but not limited to, an autoimmune disease, an inflammatory disease, a neurogenerative disease, a neuromuscular disease, or a psychiatric disease. In some embodiments, the methods and compositions provided herein are useful for treatment or prevention of the inflammation of central nervous system, including brain inflammation, peripheral nerves inflammation, neural inflammation, spinal cord inflammation, ocular inflammation, and/or other inflammation.

Examples of diseases associated with neuroinflammation or neuroinflammatory diseases which may be treated with the methods and compositions described herein include, but are not limited to, encephalitis (inflammation of the brain), encephalomyelitis (inflammation of the brain and spinal cord), meningitis (inflammation of the membranes that surround the brain and spinal cord), Guillain-Barre syndrome, neuromyotonia, narcolepsy, multiple sclerosis, myelitis, schizophrenia, acute disseminated encephalomyelitis (ADEM), accute optic neuritis (AON), transverse myelitis, neuromyelitis optica (NMO), Alzheimer's disease, Parkinson's disease, amyotrophic lateral sclerosis, frontotemporal lobar dementia, optic neuritis, neuromyelitis optica spectrum disorder (NMOSD), auto-immune encephalitis, anti-NMDA receptor encephalitis, Rasmussen's encephalitis, acute necrotizing encephalopathy of childhood (ANEC), opsoclonus-myoclonus ataxia syndrome, traumatic brain injury, Huntington's disease, depression, anxiety, migraine, myasthenia gravis, acute ischemic stroke, epilepsy, synucleinopathies, frontotemporal dementia, progressive nonfluent aphasia, semantic dementia, Nodding syndrome, cerebral ischemia, neuropathic pain, autism spectrum disorder, fibromyalgia syndrome, progressive supranuclear palsy, corticobasal degeneration, systemic lupus erythematosus, prion disease, motor neurone diseases (MND), spinocerebellar ataxia, spinal muscular atrophy, dystonia, idiopathicintracranial hypertension, nervous system disease, central nervous system disease, movement diseases, encephalopathy, peripheral neuropathy, or post-operative cognitive dysfunction.

### Cancer

As described herein, the methods and compositions provided herein can be used for preventing or treating cancer.

Cancer, tumor, or hyperproliferative disease refer to the presence of cells possessing characteristics typical of cancer-causing cells, such as uncontrolled proliferation, immortality, metastatic potential, rapid growth and proliferation rate, and certain characteristic morphological features. Cancer cells are often in the form of a tumor, but such cells may exist alone within an animal, or may be a non-tumorigenic cancer cell, such as a leukemia cell. Cancers include, but are not limited to, B cell cancer, (e.g., multiple myeloma, Diffuse large B-cell lymphoma (DLBCL), Follicular lymphoma, Chronic lymphocytic leukemia (CLL), small lymphocytic lymphoma (SLL), Mantle cell lymphoma (MCL), Marginal zone lymphomas, Burkitt lymphoma, Waldenström's macroglobulinemia, Hairy cell leukemia, Primary central nervous system (CNS) lymphoma, Primary intraocular lymphoma, the heavy chain diseases, such as, for example, alpha chain disease, gamma chain disease, and mu chain disease, benign monoclonal gammopathy, and immunocytic amyloidosis), T cell cancer (e.g., T-lymphoblastic lymphoma/leukemia, non-Hodgkin lymphomas, Peripheral T-cell lymphomas, Cutaneous T-cell lymphomas (e.g., mycosis fungoides, Sezary syndrome), Adult T-cell leukemia/lymphoma, Angioimmunoblastic T-cell lymphoma, Extranodal natural killer/T-cell lymphoma, Enteropathy-associated intestinal T-cell lymphoma (EATL), Anaplastic large cell lymphoma (ALCL), Hodgkin lymphoma), melanomas, breast cancer, lung cancer, bronchus cancer, colorectal cancer, prostate cancer, pancreatic cancer, stomach cancer, ovarian cancer, urinary bladder cancer, brain or central nervous system cancer, peripheral nervous system cancer, esophageal cancer, cervical cancer, uterine or endometrial cancer, cancer of the oral cavity or pharynx, liver cancer, kidney cancer, testicular cancer, biliary tract cancer, small bowel or appendix cancer, salivary gland cancer, thyroid gland cancer, adrenal gland cancer, osteosarcoma, chondrosarcoma, cancer of hematologic tissues, and the like. Other non-limiting examples of types of cancers applicable to the methods encompassed by the present invention include human sarcomas and carcinomas, e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, colorectal cancer, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, liver cancer, choriocarcinoma, seminoma, embryonal carcinoma, Wilms' tumor, cervical cancer, bone cancer, brain tumor, testicular cancer, lung carcinoma, small cell lung carcinoma (SCLC), bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma; leukemias, e.g., acute lymphocytic leukemia and acute myelocytic leukemia (myeloblastic, promyelocytic, myelomonocytic, monocytic and erythroleukemia); chronic leukemia (chronic myelocytic (granulocytic) leukemia and chronic lymphocytic leukemia); and polycythemia vera, lymphoma (Hodgkin's disease and non-Hodgkin's disease), multiple myeloma, Waldenstrom's macroglobulinemia, and heavy chain disease. In some embodiments, cancers are epithelial in nature and include but are not limited to, bladder cancer, breast cancer, cervical cancer, colon cancer, gynecologic cancers, renal cancer, laryngeal cancer, lung cancer, oral cancer, head and neck cancer, ovarian cancer, pancreatic cancer, prostate cancer, or skin cancer. In other embodiments, the cancer is breast cancer, prostate cancer, lung cancer, or colon cancer. In still other embodiments, the epithelial cancer is non-small-cell lung cancer, nonpapillary renal cell carcinoma, cervical carcinoma, ovarian carcinoma (e.g., serous ovarian carcinoma), or breast carcinoma. The epithelial cancers may be characterized in various other ways including, but not limited to, serous, endometrioid, mucinous, clear cell, Brenner, or undifferentiated.

### Exemplary Embodiments

1. A composition comprising at least two bacterial strains selected from:
Bifidobacterium longum, Bifidobacterium breve, and Bifidobacterium infantis, optionally further comprising at least one excipient.
   2. The composition of 1, wherein the composition comprises Bifidobacterium longum, Bifidobacterium breve, and Bifidobacterium infantis.
   3. The composition of 1 or 2, wherein
(a) at least about 10% but no more than about 70% of the bacteria in the composition are Bifidobacterium Longum;
(b) at least about 1% but no more than about 50% of the bacteria in the composition are Bifidobacterium breve; and/or
(c) at least about 10% but no more than about 70% of the bacteria in the composition are Bifidobacterium infantis,
wherein the percent bacteria refers to the percentage of the colony forming units (CFU) of said bacteria relative to the total CFU of bacteria in the composition.
   4. The composition of any one of 1-3, wherein the bacteria in the composition comprise about 50% Bifidobacterium Longum, about 10% Bifidobacterium breve, and about 40% Bifidobacterium infantis, wherein the percent bacteria refers to the percentage of the CFU of said bacteria relative to the total CFU of bacteria in the composition.
   5. The composition of any one of 1-4, wherein the composition comprises at least about 1×10⁴ but no more than about 600×10⁹ total CFU of bacteria per gram of the composition.
   6. The composition of any one of 1-5, wherein the composition comprises at least about 180×10⁹ but no more than about 270×10⁹ total CFU of bacteria per gram of the composition, preferably wherein the composition comprises about 250×10⁹ total CFU of bacteria per gram of the composition.
   7. The composition of any one of 1-6, further comprising at least one immunomodulatory agent.
   8. The composition of 7, wherein the at least one immunomodulatory agent is selected from: a steroid (e.g., prednisone, methylprednisolone, dexamethasone), colchicine, hydroxychloroquine, sulfasalazine, dapsone, methotrexate, mycophenolate mofetil, azathioprine, an IL-1 inhibitor (e.g., anakinra, canakinumab, rilonacept), a TNF inhibitor (e.g., infliximab, adalimumab, golimumab, etanercept, certolizumab), an IL-6 inhibitor (e.g., tocilizumab, sarilumab), eculizumab, a CD20 inhibitor (e.g., rituximab), belimumab, cyclosporine, abatacept, an IL-17 inhibitor (e.g., secukinumab, ixekizumab, brodalumab), an IL-23 inhibitor (e.g., guselkumab), an IL-12/IL-23 inhibitor (e.g., ustekinumab), an IL-5 inhibitor (e.g., mepolizumab, reslizumab, benralizumab), an IL-4/IL-13 inhibitor (e.g., dupilumab), omalizumab, vedolizumab, N-acetylcysteine (NAC), and a JAK inhibitor (e.g., tofacitinib, upadacitinib, baricitinib).
   9. The composition of any one of 1-8, wherein the composition increases the production or secretion of IL-10, G-CSF and/or GRO-alpha by NK cells.
   10. A composition comprising at least two bacterial strains selected from: Streptococcus thermophiles, Lactobacillus acidophilus, Lactobacillus plantarum, and Lactobacillus paracasei, optionally further comprising at least one excipient.
   11. The composition of 10, wherein the composition comprises Streptococcus thermophiles, Lactobacillus acidophilus, Lactobacillus plantarum, and Lactobacillus paracasei.
   12. The composition of 10 or 11, wherein
(a) at least about 10% but no more than about 60% of the bacteria in the composition are Streptococcus thermophiles;
(b) at least about 1% but no more than about 50% of the bacteria in the composition are Lactobacillus acidophilus;
(c) at least about 10% but no more than about 70% of the bacteria in the composition are Lactobacillus plantarum; and/or
(d) at least about 1% but no more than 40% of the bacteria in the composition are Lactobacillus paracasei,
wherein the percent bacteria refers to the percentage of the CFU of said bacteria relative to the total CFU of bacteria in the composition.
   13. The composition of any one of 10-12, wherein the bacteria in the composition comprise about 30% Streptococcus thermophiles, about 20% Lactobacillus acidophilus, about 40% Lactobacillus plantarum, and about 10% Lactobacillus paracasei, wherein the percent bacteria refers to the percentage of the CFU of said bacteria relative to the total CFU of bacteria in the composition.
   14. The composition of any one of 10-13, wherein the composition comprises at least about 1×10⁴ but no more than about 600×10⁹ total CFU of bacteria per gram of the composition.
   15. The composition of any one of 10-14, wherein the composition comprises about 250×10⁹ total CFU of bacteria per gram of the composition.
   16. The composition of any one of 10-15, further comprising at least one cancer therapy.
   17. The composition of 16, wherein the at least one cancer therapy is chemotherapy, radiotherapy, or immunotherapy.
   18. The composition of 17, wherein the chemotherapy is CDDP, Paclitaxel (PTX), and/or N-acetylcysteine (NAC).
   19. The composition of 17, wherein the immunotherapy inhibits an immune checkpoint.
   20. The composition of 19, wherein the immune checkpoint is selected from CTLA-4, PD-1, VISTA, B7-H2, B7-H3, PD-L1, B7-H4, B7-H6, ICOS, HVEM, PD-L2, CD160, gp49B, PIR-B, KIR family receptors, TIM-1, TIM-3, TIM-4, LAG-3, GITR, 4-IBB, OX-40, BTLA, SIRPalpha (CD47), CD48, 2B4 (CD244), B7.1, B7.2, ILT-2, ILT-4, TIGIT, HHLA2, butyrophilins, and A2aR.
   21. The composition of any one of 17, 19, and 20, wherein the immunotherapy is selected from: atezolizumab, avelumab, durvalumab, ipilimumab, nivolumab, pembrolizumab, pidilizumab, AMP-224, AMP-514, BGB-A317, STI-A1110, TSR-042, RG-7446, BMS-936559, MEDI-4736, MSB-0010718C, AUR-012, and STI-A1010.
   22. The composition of any one of 10-21, wherein the composition increases the production or secretion by NK cells:
(a) IFN-γ, optionally wherein the composition increases the production or secretion of IFN-γ relative to the production or secretion of IL-10; and/or
(b) MCP-1.
   23. The composition of any one of 1-22, further comprising an agent capable of activating and/or expanding NK cells, optionally wherein the agent is a cytokine or an osteoclast cell.
   24. The composition of any one of 1-23, further comprising an NK cell or a super-charged NK cell.
   25. The composition of 24, wherein the NK cell or the super-charged NK cell is autologous or allogeneic to the subject.
   26. The composition of any one of 1-25, wherein the composition is in a pharmaceutical or nutraceutical composition (e.g., a dietary supplement, a tablet, a capsule).
   27. The composition of any one of 1-26, wherein the composition increases the production or secretion of at least one cytokine by NK cells.
   28. The composition of 27, wherein the at least one cytokine is selected from: IL-10, IFN-γ, TGF-α, IL-6, IL-1ra, IL-1B, IL-12p70, G-CSF, IL-8, GM-CSF, RANTES, Eotaxin, IP-10, IL-13, MCP-1, GRO-alpha, and IL-17.
   29. The composition of any one of 1-28, wherein the composition is administered by intravenous, intratumoral, oral, or rectal administration.
   30. A kit comprising the composition of any one of 1-29.
   31. A method of preventing or treating an inflammatory disease or an autoimmune disease in a subject, comprising administering to the subject a composition of any one of 1-9 and 23-29, optionally further comprising administering to the subject at least one immunomodulatory agent.
   32. The method of 31, wherein the the inflammatory disease or an autoimmune disease is selected from: type 1 diabetes mellitus, arthritis (e.g., rheumatoid arthritis, idiopathic arthritis, active enthesitis-related arthritis), psoriasis, psoriatic arthristis, hidradenitis suppurativa, multiple sclerosis, systemic lupus erythematosus, inflammatory bowel disease (e.g., crohn's disease, ulcerative colitis), Addison's disease, Graves' disease, Sjogren's syndrome, Hashimoto's thyroiditis, myasthenia gravis, allergy, Amyotrophic lateral sclerosis (ALS), and autoimmune vasculitis.
   33. The method of 31 or 32, wherein the inflammatory disease is ALS.
   34 The method of 33, further comprising administering to the subject an additional therapy that treats ALS, optionally selected from riluzole, edaravone, masitinib, retigabine, and tamoxifen.
   35. The method of any one of 31-34, wherein the method increases the production or secretion of at least one cytokine in the subject.
   36. The method of 35, wherein the at least one cytokine is selected from: IL-10, IFN-γ, TGF-α, IL-6, IL-1ra, IL-1B, IL-12p70, G-CSF, IL-8, GM-CSF, RANTES, Eotaxin, IP-10, IL-13, MCP-1, GRO-alpha, and IL-17.
   37. The method of 35 or 36, wherein the at least one cytokine is selected from IL-10, G-CSF, and GRO-alpha, optionally IL-10.
   38. A method of increasing the production or secretion of IL-10, G-CSF, and/or GRO-alpha in a subject, the method comprising administering to the subject a composition of any one of 1-9 and 23-29.
   39. A method of increasing the production or secretion of IL-10, G-CSF, and/or GRO-alpha by NK cells, the method comprising contacting the NK cells with a composition of any one of 1-9 and 23-29.
   40. The method of 39, wherein the method is performed in vitro, ex vivo, or in vivo.
   41. A method of preventing or treating a cancer in a subject, the method comprising administering to the subject a composition of any one of 10-29.
   42. The method of 41, further comprising administering conjointly to the subject at least one cancer therapy.
   43. The method of 42, wherein the at least one cancer therapy is chemotherapy, radiotherapy, or immunotherapy.
   44. The method of 43, wherein the chemotherapy is CDDP, Paclitaxel (PTX), and/or N-acetylcysteine (NAC).
   45. The method of 43, wherein the immunotherapy inhibits an immune checkpoint.
   46. The method of 45, wherein the immune checkpoint is selected from CTLA-4, PD-1, VISTA, B7-H2, B7-H3, PD-L1, B7-H4, B7-H6, ICOS, HVEM, PD-L2, CD160, gp49B, PIR-B, KIR family receptors, TIM-1, TIM-3, TIM-4, LAG-3, GITR, 4-IBB, OX-40, BTLA, SIRPalpha (CD47), CD48, 2B4 (CD244), B7.1, B7.2, ILT-2, ILT-4, TIGIT, HHLA2, butyrophilins, and A2aR.
   47. The method of any one of 43, 45, and 46, wherein the immunotherapy is selected from: atezolizumab, avelumab, durvalumab, ipilimumab, nivolumab, pembrolizumab, pidilizumab, AMP-224, AMP-514, BGB-A317, STI-A1110, TSR-042, RG-7446, BMS-936559, MEDI-4736, MSB-0010718C, AUR-012, and STI-A1010.
   48. The method of any one of 41-47, further comprising administering conjointly to the subject an agent capable of activating and/or expanding NK cells, optionally wherein the agent is a cytokine or an osteoclast cell.
   49. The method of any one of 41-48, further comprising administering to the subject an NK cell or a super-charged NK cell.
   50. The method of 49, wherein the NK cell or the super-charged NK cell is autologous or allogeneic to the subject.
   51. The method of any one of 41-50, wherein the cancer is selected from multiple myeloma, prostate cancer, stomach cancer, bladder cancer, esophageal cancer, cervical cancer, liver cancer, kidney cancer, bone cancer, brain cancer, leukemia, head and neck cancer, oral cancer, pancreatic cancer, lung cancer, colon cancer, melanoma, breast cancer, ovarian cancer, and glioblastoma.
   52. The method of any one of 41-51, wherein the cancer is selected from oral cancer, pancreatic cancer, lung cancer, colon cancer, melanoma, breast cancer, ovarian cancer, and glioblastoma.
   53. The method of any one of 41-52, wherein the composition is administered at least twice to the subject.
   54. The method of any one of 41-53, wherein the method inhibits proliferation of a cancer cell and/or increases cancer cell differentiation.
   55. The method of any one of 41-54, wherein the method increases the production or secretion of at least one cytokine in the subject.
   56. The method of 55, wherein the at least one cytokine is selected from: IL-10, IFN-γ, TGF-α, IL-6, IL-1ra, IL-1B, IL-12p70, G-CSF, IL-8, GM-CSF, RANTES, Eotaxin, IP-10, IL-13, MCP-1, GRO-alpha, and IL-17.
   57. The method of 55 or 56, wherein the composition increases the production of IFN-γ, optionally wherein the composition increases the production of IFN-γ relative to the production of IL-10.
   58. The method of any one of 31-57, wherein the subject is healthy or diseased (e.g., afflicted with an inflammatory disease, autoimmune disease, or cancer).
   59. The method of any one of 31-58, wherein the composition is administered by intravenous, intratumoral, oral, or rectal administration.
   60. The method of any one of 31-59, wherein the subject is a mammal, preferably wherein the mammal is a mouse or human.

### EXAMPLES

### Example 1: Materials and Methods for Examples 2 and 3

### Cell Lines, Reagents, and Antibodies

Human immune cells were cultured in RPMI 1640, supplemented with 10% fetal bovine serum (FBS) (Gemini Bio-Products, CA). Oral squamous carcinoma stem cells (OSCSCs) were isolated from oral cancer patient tongue tumors at UCLA School of Medicine and cultured in RPMI 1640, supplemented 10% FBS (Gemini Bio-Products, CA), 1.4% antibiotic antimycotic, 1% sodium pyruvate, 1.4% MEM non-essential amino acids, 1% L-glutamine, 0.2% gentimicin (Gemini Bio-products, CA) and 0.15% sodium bicarbonate (Fisher Scientific, PA).

Antibodies to CD16 were purchased from Biolegend (San Diego, CA, USA). Recombinant IL-2 was obtained from NIH-BRB. Antibodies against isotype control, MHC-I, CD45 (human), CD45 (mouse), CD3, CD16, CD56, CD8, HLADR, and CD11b were purchased from Biolegend (San Diego, CA). Human NK purification kits were obtained from Stem Cell Technologies (Vancouver, BC, Canada).

Human monocytes/osteoclasts were cultured in alpha-MEM medium (Life Technologies, CA), supplemented with 10% FBS, and penicillin-streptomycin (Gemini Bio-Products, CA). Human M-CSF (Biolegend, CA) and soluble RANKL (PeproTech, NJ) were dissolved in alpha-MEM and stored at −80° C.

### Bacteria Selection and Preparation

AJ2 is a combination of 8 different strains of gram positive probiotic bacteria (Streptococcus thermophiles, Bifidobacterium longum, Bifidobacterium breve, Bifidobacterium infantis, Lactobacillus acidophilus, Lactobacillus plantarum, Lactobacillus paracasei, KE99, and Lactobacillus bulgaricus) used to induce differentiation of stem cells.

Different strains of probiotic bacteria were tested for their ability to induce IFN-g as well as a number of important cytokines, chemokines and growth factors. To assess the levels of each strain used in the combination we used an activation index established in the lab for the NK cells which consisted of the use of a specific ratio of several important cytokines and chemokines that prevented auto-immunity while enhancing significant activation of NK cells under pathologic conditions. Therefore, the strains were selected to: 1) provide regulated activation of NK cells when no activation of NK cells is desired; 2) when activated by cytokines and/or cross-linking of the important receptors which occurs during functional activation of NK cells in cancer is to induce maximal activation of NK cells; and 3) provide diversity for gut microflora. These criteria allow the use bacteria to regulate the gut mucosal immunity in such a way that we will increase NK activation when needed during infections or malignancies, and/or provide sufficient induction of necessary cytokines when the bacterial composition is provided as a nutraceutical composition to a healthy individual. In other words when there is no need for function of NK cells bacteria will regulate NK function to not induce inflammation; however, during need which occurs under the pathological conditions NK cells are triggered to function at the maximal levels. Such coordinated regulation of NK cell function should be effective in halting unwanted inflammation while providing effective and maximal immunity during disease.

AJ2, an AJ3 composition comprising e.g., Bifidobacterium, or an AJ4 composition comprising e.g., Lactobacillus was weighed and resuspended in RPMI Medium 1640 containing 10% FBS at a concentration of 10 mg per 1 mL. The bacteria were thoroughly vortexed, then sonicated on ice for 15 seconds, at 6 to 8 amplitude. Sonicated samples were then incubated for 30 seconds on ice. After every five pulses, a sample was taken to observe under the microscope until at least 80 percent of cell walls were lysed. It was determined that approximately 20 rounds of sonication/incubation on ice, were conducted to achieve complete sonication. Finally, the sonicated samples were aliquoted and stored in a −80 degrees Celsius freezer. Sonication of bacteria is not required or necessary to render its activities presented herein.

Purification of NK Cells from the Peripheral Blood

Written informed consents, approved by UCLA Institutional Review Board (IRB), were obtained from healthy blood donors, and all procedures were approved by the UCLA-IRB. Peripheral blood was separated using Ficoll-Hypaque centrifugation, after which the white, cloudy layer, containing peripheral blood mononuclear cells (PBMC), was harvested, washed and re-suspended in RPMI 1640 (Invitrogen by Life Technologies, CA) supplemented with 10% FBS and plated on plastic tissue culture dishes. After 1-2 hours of incubation, non-adherent, human peripheral blood lymphocytes (PBL) were collected. NK cells were negatively selected and isolated from PBLs using the Easy SepR Human NK cell enrichment kit purchased from Stem Cell Technologies (Vancouver, BC, Canada). Isolated NK cells were stained with anti-CD16 antibody, to measure NK cell purity using flow cytometric analysis. The isolated NK cell population was greater than 90% purity. Purified NK cells were cultured in RPMI Medium 1640 supplemented with 10% FBS (Gemini Bio-Products, CA), 1% antibiotic antimycotic, 1% sodium pyruvate, and 1% MEM non-essential amino acids (Invitrogen, Life Technologies, CA).

### NK Cell Supernatants Used for Stem Cell Differentiation

As described above, human NK cells were purified from PBMCs of healthy donors. NK cells were left untreated, treated with sAJ2 at 1:3 (NK: sAJ2), and/or a combination of anti-CD16 mAb (3 μg/mL) and IL-2 (1,000 U/mL) for 18 hours before supernatants were removed and used for differentiation experiments. The amounts of IFN-γ produced by activated NK cells were assess with IFN-γ ELISA (Biolegend, CA, USA). OSCSCs were differentiated with gradual daily addition of increasing amounts of NK cell supernatants (of corresponding treatments). On average, to induce differentiation, a total of 4,500 pg of IFN-γ containing supernatants, obtained from IL-2+anti-CD16 mAb+sAJ2-treated NK cells, was added for 4 days to induce differentiation and resistance of OSCSCSs to NK cell-mediated cytotoxicity. Afterwards, target cells were washed with 1×PBS, detached and used for experiments.

Purification of Monocytes from the Peripheral Blood

Written informed consents, approved by UCLA Institutional Review Board (IRB) were obtained from healthy blood donors, and all procedures were approved by the UCLA-IRB. Peripheral blood was separated using Ficoll-Hypaque centrifugation, after which the white, cloudy layer, containing peripheral blood mononuclear cells (PBMC), was harvested, washed and re-suspended in RPMI 1640 (Invitrogen by Life Technologies, CA) supplemented with 10% FBS and plated on plastic tissue culture dishes. After 1-2 hours of incubation, the adherent subpopulation of PBMCs was detached from the tissue culture plates. Monocytes were purified using the EasySep® Human monocyte cell enrichment kit obtained from Stem Cell Technologies (Vancouver, BC, Canada). Based on flow cytometric analysis of CD14 the antibody-stained, enriched monocyte cells, the monocyte population was found to have greater than a 95% purify.

### Generation of Osteoclasts (HOCS)

Osteoclasts were generated from PBMC-purified monocytes and cultured in alpha-MEM medium, containing M-CSF (25 ng/ml) and RANK Ligand (RANKL) (25 ng/ml), for 21 days. Medium was refreshed every 3 days with fresh alpha-MEM, containing M-CSF (25 ng/ml) and RANKL (25 ng/ml).

### Surface Staining

1×10⁵ cells from each condition were stained in 100 μl of cold 1% PBS-BSA with pre-determined optimal concentration of PE conjugated antibodies, as detailed in the experiments, and incubated at 4° C. for 30 minutes. Then, cells were washed and resuspended in 1% PBS-BSA. The Epics C (Coulter) flow cytometer was used for cellular surface analysis.

### ⁵¹CR Release Cytotoxicity Assay

⁵¹Cr was purchased from Perkin Elmer (Santa Clara, CA). Standard ⁵¹Cr release cytotoxicity assays were used to determine NK cell cytotoxic function in the experimental cultures and the sensitivity of target cells to NK cell mediated lysis. The effector cells (1×10⁵ NK cells/well) were aliquoted into 96-well round-bottom microwell plates (Fisher Scientific, Pittsburgh, PA) and titrated at four to six serial dilutions. The target cells (5×10⁵ OSCSCs) were labeled with 50 μCi 51Cr (Perkin Elmer, Santa Clara, CA) and chromated for 1 hour. Following incubation, target cells were washed twice to remove excess unbound ⁵¹Cr. ⁵¹Cr-labeled target cells were aliquoted into the 96-well round bottom microwell plates containing effector cells at a concentration of 1×10⁴ cells/well at a top effector: target (E:T) ratio of 5:1. Plates were centrifuged and incubated for a period of 4 hours. After a 4-hour incubation period, the supernatants were harvested from each sample and counted for released radioactivity using the gamma counter. Total (containing ⁵¹Cr-labeled target cells) and spontaneous (supernatants of target cells alone) release values were measured and used to calculate the percentage specific cytotoxicity. The percentage specific cytotoxicity was calculated using the following formula:

% Cytotoxicity=Experimental cpm−spontaneous cpm/Total cpm−spontaneous cpm

LU 30/10⁶ is calculated by using the inverse of the number of effector cells needed to lyse 30% of target cells×100.

### Enzyme-Linked Immunosorbent Assays (Elisas) and Multiplex Cytokine Assay

Human ELISA kits for IFN-γ and IL-10 were purchased from Biolegend (San Diego, CA). ELISA was performed to detect the level of IFN-γ and IL-10 produced from cell cultures. The assay was conducted as described in the manufacturer's protocol. Briefly, 96-well EIA/RIA plates were coated with diluted capture antibody corresponding to target cytokine and incubated overnight at 4° C. After 16-18 hours of incubation, the plates were washed 4 times with wash buffer (0.05% Tween in 1×PBS) and blocked with assay diluent (1% BSA in 1×PBS). The plates were incubated for 1 hour at room temperature, on a plate shaker at 200 rpm; plates were washed 4 times following incubation. Then, 100 μL of standards and samples collected from each culture were added to the wells and incubated for 2 hours at room temperature, on the plate shaker at 200 rpm. After incubation, plates were washed 4 times, loaded with detection antibody, and incubated for 1 hour at room temperature, on the plate shaker at 200 rpm. After 1 hour of incubation, the plates were washed 4 times; wells were loaded with Avidin-HRP solution and incubated for 30 minutes at room temperature, on the plate shaker at 200 rpm. After washing the plates 5 times with wash buffer; 100 μL of TMB substrate solution was added to the wells and plates were incubated in the dark until they developed a desired blue color (or up to 30 minutes). Then, 100 μL of stop solution (2N H₂SO₄) was added per well to stop the reaction. Finally, plates were read in a microplate reader, at 450 nm to obtain absorbance values (Biolegend, ELISA manual).

The levels of cytokines and chemokines were examined by multiplex assay, which was conducted as described in the manufacturer's protocol for each specified kit. Analysis was performed using a Luminex multiplex instrument (MAGPIX, Millipore, Billerica, MA) and data was analyzed using the proprietary software (xPONENT 4.2, Millipore, Billerica, MA).

### Statistical Analysis

An unpaired, two-tailed student t-test was performed for the statistical analysis of two groups. One-way ANOVA with a Bonferroni post-test was used to compare more than two groups.

### Example 2: Cloned Bacterial Strains Demonstrate Differential Trigger of Cytokines and Chemokines

Natural killer (NK) cells are innate lymphoid cells (ILCs) that contribute to immunity through direct lysis of tumor or virus infected cells and though the secretion of immune-regulatory cytokines. The overall impact of NK cells in a given setting depends on their activation state and which cytokines they produce.

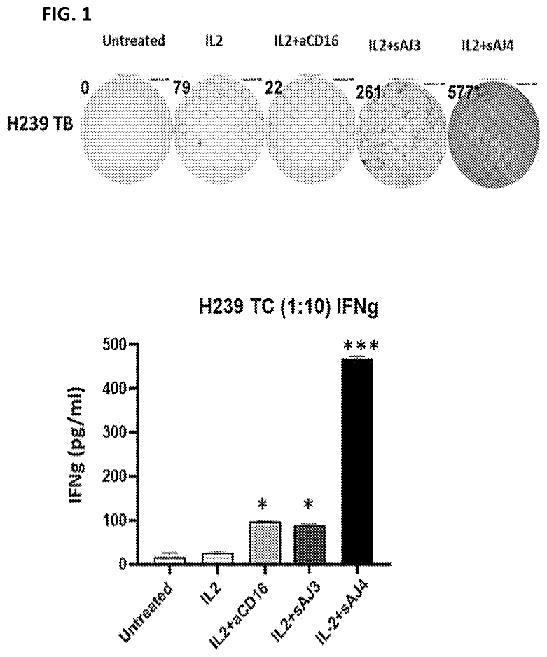

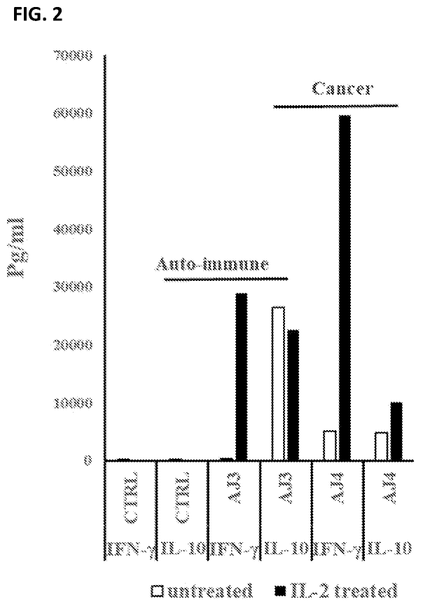

We tested the ability of NK cells to be activated by a number of different bacterial strains (Tables 2-10 and FIGS. 1-2). It was observed herein that the bacterial strains tested herein activated NK cells and resulted in differential trigger of cytokines and chemokines. It was noted that Lactobacillus and Streptococcus species preferentially induced higher IFN-g and MCP-1 (proinflammatory cytokines that increase the immune response to cancer), whereas Bifidobacterium species triggered higher IL-10 and G-CSF (inhibitory cytokines that downregulate inflammation in inflammatory/autoimmune diseases) secretion and regulated IFN-g secretion. In addition, Bifidobacterium species induced higher levels of G-CSF and Gro-alpha suggesting that these species would preferentially stimulate the generation and activation of subsets of myeloid populations that are known to inhibit the functions of NK and T cells (such as PMNs and MDSCs), whereas Lactobacillus will stimulate lymphoid populations. Indeed, when NK cells were cultured with monocytes and treated with either Bifidobacterial or Lactobcillus bacteria, they exhibited the same dichotomy (see e.g., Table 10). Accordingly, these observations indicate that the one or more Bifidobacterial species are particularly useful for use in auto-immune patients where they need higher regulation of inflammation by the increased secretion of IL-10, whereas the one or more Lactobacillus and Streptococcus species are useful for the treatment of cancer patients. Notably, healthy individuals would benefit from a composition (e.g., nutraceutical composition, e.g., dietary supplement) comprising both bacterial strains by improving the immune response e.g., to infections. The data presented herein highlight the utility of two distinct probiotic bacterial formulations: bacterial species of Lactobacillus and Streptococcus for use as adjuvant supplementation for cancer treatment; or Bifidobacterium species for use in auto immune disease patients, respectively.

### Example 3: Exemplary Formulations of Bacterial Compositions

### AJ3

The AJ3 bacterial composition comprising Bifidobacterium Longum, Bifidobacterium breve, and Bifidobacterium infantis was effective in inducing secretion of a high level of IL-10 and G-CSF that are useful for downregulating inflammation in patients afflicted with autoimmune diseases. It is notable that the AJ3 bacterial compositions comprising either intact or sonicated bacteria were equally effective. The effective AJ3 composition comprised: (i) about 40-60% of Bifidobacterium Longum, (ii) about 5-20% of Bifidobacterium breve, and (iii) about 30-50% of Bifidobacterium infantis. An exemplary formulation for AJ3 is shown in Table 1.

TABLE 1
Exemplary formulation of AJ3

| Bacteria | Percent of total | 250 × 10⁹ CFU | g |
|---|---|---|---|
| B. Longum | 50% | 125 × 10⁹ | 0.78 |
| B. breve | 10% | 25 × 10⁹ | 0.09 |
| B. infantis | 40% | 100 × 10⁹ | 0.22 |
| | 100% | 250 × 10⁹ | 1.09 |

### AJ4

The AJ4 bacterial composition comprising Streptococcus thermophiles, Lactobacillus acidophilus, Lactobacillus plantarum, and Lactobacillus paracasei was effective in inducing secretion of IFN-g and MCP-1, proinflammatory cytokines that increase the immune response to cancer. It is notable that the AJ4 bacterial compositions comprising either intact or sonicated bacteria were equally effective. The effective AJ4 composition comprised: (i) about 20-40% of Streptococcus thermophiles, (ii) about 10-30% of Lactobacillus acidophilus, (iii) about 30-50% Lactobacillus plantarum, and (iv) about 5-20% Lactobacillus paracasei. An exemplary formulation for AJ4 is shown in Table 2.

TABLE 2
Exemplary formulation of AJ4

| Bacteria | Percent of total | 250 × 10⁹ CFU | 1 g |
|---|---|---|---|
| S. thermophilus | 30% | 75 × 10⁹ | 0.54 |
| L. acidophilus | 20% | 50 × 10⁹ | 0.2 |
| L. paracasaei | 10% | 25 × 10⁹ | 0.2 |
| L. plantarum | 40% | 100 × 10⁹ | 0.05 |
| | 100% | 250 × 10⁹ | 0.99 |

Table 3. Production of Cytokines, Growth Factors, and Chemokines by NK Cells Treated with Bacterial Strains

Purified NK cells from healthy donors were left untreated or treated with IL-2 (1000 units/ml) or the combination of anti-CD16mAb (3 μg/ml) and IL-2 (1000 units/ml) in the presence or absence of probiotic bacteria sAJ2 at 1:5 ratio (NK: sAJ2) for 18 hours. Afterwards, the levels of cytokines, growth factors and chemokines were determined using Bio-Plex Pro Human Cytokine 27-plex Array Kit.

| | Treat- | IL- | IFN- | IL- | TNF- | IL- | IL- | IL- | G- | IL- | GM- | | Eo- | IP- | IL- | MCP- | IL- |
|---|---|---|---|---|---|---|---|---|---|---|---|---|---|---|---|---|---|
| Bacteria | ments | 6 | γ | 1ra | α | 1B | 10 | 12p70 | CSF | 8 | CSF | RANTES | taxin | 10 | 13 | 1 | 17 |
| Control | NK | 38 | 34 | 41 | 44 | 4 | 6 | 2 | 28 | 281 | 86 | 2564 | 5 | 64 | 2 | 258 | 33 |
| | NK + IL-2 | 55 | 99 | 439 | 137 | 10 | 6 | 5 | 54 | 1148 | 100 | 3151 | 12 | 137 | 5 | 525 | 51 |
| | NK + IL- | 39 | 138 | 466 | 166 | 22 | 8 | 7 | 65 | 1480 | 102 | 2536 | 20 | 114 | 5 | 305 | 58 |
| | 2 + inti- | | | | | | | | | | | | | | | | |
| sAJ2 | NK | 20292 | 222 | 308 | 9634 | 1041 | 200 | 83 | 725 | 12107 | 121 | 1797 | 23 | 40 | 7 | 526 | 54 |
| | NK + IL-2 | 6034 | 894 | 425 | 2166 | 454 | 38 | 70 | 221 | 2430 | 113 | 1440 | 17 | 34 | 7 | 104 | 50 |
| | NK + IL- | 7944 | 1219 | 618 | 3883 | 839 | 51 | 72 | 350 | 6503 | 201 | 2704 | 35 | 51 | 23 | 107 | 72 |
| | 2 + inti- | | | | | | | | | | | | | | | | |
| S. ther- | NK | 10222 | 785 | 229 | 18892 | 1381 | 63 | 515 | 149 | 3326 | 131 | 2580 | 40 | 64 | 9 | 502 | 74 |
| mophilus | NK + IL-2 | 1773 | 1188 | 465 | 2413 | 390 | 17 | 132 | 87 | 1341 | 124 | 2197 | 21 | 45 | 9 | 169 | 65 |
| | NK + IL- | 2475 | 2594 | 434 | 4917 | 642 | 23 | 163 | 101 | 1841 | 177 | 2097 | 29 | 58 | 15 | 332 | 57 |
| | 2 + inti- | | | | | | | | | | | | | | | | |
| B. breve | NK | 9726 | 99 | 196 | 2904 | 200 | 127 | 10 | 363 | 3975 | 101 | 1662 | 15 | 32 | 5 | 265 | 53 |
| | NK + IL-2 | 3402 | 284 | 470 | 1868 | 165 | 36 | 21 | 138 | 1912 | 127 | 1988 | 22 | 35 | 9 | 72 | 57 |
| | NK + IL- | 7261 | 844 | 565 | 1498 | 421 | 68 | 26 | 289 | 4929 | 180 | 3934 | 29 | 55 | 28 | 253 | 72 |
| | 2 + inti- | | | | | | | | | | | | | | | | |
| B. longum | NK | 60895 | 158 | 312 | 5880 | 813 | 298 | 42 | 938 | 9507 | 131 | 2325 | 32 | 38 | 9 | 80 | 74 |
| | NK + IL-2 | 7287 | 671 | 472 | 2896 | 408 | 52 | 47 | 244 | 2195 | 135 | 2189 | 33 | 46 | 11 | 33 | 73 |
| L. aci- | NK + IL- | 2916 | 1282 | 461 | 4240 | 498 | 68 | 33 | 386 | 2906 | 158 | 1088 | 28 | 42 | 19 | 75 | 69 |
| dophilus | 2 + inti- | | | | | | | | | | | | | | | | |
| | NK | 10357 | 366 | 181 | 6858 | 819 | 62 | 299 | 141 | 6879 | 110 | 2553 | 16 | 77 | 5 | 400 | 63 |
| | NK + IL-2 | 2095 | 1329 | 487 | 1151 | 335 | 17 | 136 | 89 | 1116 | 127 | 2353 | 21 | 51 | 8 | 112 | 63 |
| | NK + IL- | 3684 | 2715 | 493 | 5663 | 654 | 24 | 155 | 102 | 2004 | 199 | 3990 | 31 | 69 | 23 | 289 | 73 |
| | 2 + inti- | | | | | | | | | | | | | | | | |
| L. | NK | 2518 | 144 | 112 | 868 | 293 | 22 | 25 | 77 | 4501 | 121 | 3804 | 7 | 88 | 9 | 609 | 49 |
| bulgaricas | NK + IL-2 | 837 | 381 | 449 | 439 | 123 | 12 | 21 | 69 | 1997 | 120 | 4737 | 15 | 78 | 10 | 313 | 55 |
| | NK + IL- | 685 | 306 | 383 | 1064 | 141 | 9 | 5 | 59 | 1819 | 136 | 4062 | 6 | 72 | 8 | 305 | 50 |
| | 2 + inti- | | | | | | | | | | | | | | | | |
| L. | NK | 3029 | 186 | 129 | 3809 | 320 | 70 | 70 | 106 | 3716 | 99 | 1069 | 4 | 53 | 8 | 506 | 40 |
| paracasei | NK + IL-2 | 7285 | 2112 | 551 | 6776 | 523 | 47 | 125 | 138 | 3983 | 157 | 6468 | 27 | 90 | 15 | 337 | 63 |
| | NK + IL- | 6867 | 2145 | 573 | 10128 | 721 | 43 | 123 | 153 | 6385 | 215 | >22609 | 40 | 92 | 27 | 427 | 90 |
| | 2 + inti- | | | | | | | | | | | | | | | | |
| L. plan- | NK | 9500 | 282 | 323 | 13887 | 645 | 85 | 130 | 236 | 17756 | 122 | 22609 | 36 | 140 | 8 | 958 | 78 |
| taram | NK + IL-2 | 4212 | 5484 | 500 | 9223 | 466 | 43 | 184 | 129 | 3415 | 181 | 1923 | 36 | 115 | 14 | 427 | 73 |
| | NK + IL- | 3679 | 3087 | 575 | 7942 | 714 | 37 | 155 | 125 | 3247 | 216 | 4147 | 40 | 92 | 20 | 335 | 78 |
| | 2 + inti- | | | | | | | | | | | | | | | | |
| B. | NK | 6136 | 275 | 179 | 5728 | 249 | 55 | 66 | 145 | 4265 | 123 | 2234 | 18 | 57 | 1 | 451 | 56 |
| Infantis | NK + IL-2 | 6618 | 3073 | 459 | 5103 | 422 | 41 | 115 | 190 | 5090 | 163 | 3225 | 19 | 113 | 19 | 382 | 72 |
| | NK + IL- | 3927 | 1250 | 434 | 5287 | 365 | 34 | 47 | 151 | 3557 | 178 | 2646 | 32 | 88 | 22 | 360 | 65 |
| | 2 + inti- | | | | | | | | | | | | | | | | |

TABLE 4
Production of cytokines, growth factors, and chemokines
by NK cells treated with bacterial strains

| | IFN- | IL- | G- | IP- | MCP- | IFN-g/ | IL-10/ |
|---|---|---|---|---|---|---|---|
| Bacteria | γ | 10 | CSF | 10 | 1 | IL-10 | IFN-g |
| Control | 34 | 6 | 28 | 64 | 258 | 6 | 0.18 |
| | 99 | 6 | 54 | 137 | 525 | 17 | 0.06 |
| | 138 | 8 | 65 | 114 | 305 | 17 | 0.06 |
| sAJ2 | 222 | 200 | 725 | 40 | 526 | 1 | 0.90 |
| | 894 | 38 | 221 | 34 | 104 | 24 | 0.04 |
| | 1219 | 51 | 350 | 51 | 107 | 24 | 0.04 |
| S. | 785 | 63 | 149 | 64 | 502 | 12 | 0.08 |
| thermophilus | 1188 | 17 | 87 | 45 | 169 | 70 | 0.01 |
| | 2594 | 23 | 101 | 58 | 332 | 113 | 0.01 |
| B. breve | 99 | 127 | 363 | 32 | 265 | 1 | 1.28 |
| | 284 | 36 | 138 | 35 | 72 | 8 | 0.13 |
| | 844 | 68 | 289 | 55 | 153 | 12 | 0.08 |
| B. longum | 158 | 298 | 938 | 38 | 80 | 1 | 1.89 |
| | 671 | 52 | 244 | 46 | 33 | 13 | 0.08 |
| | 1282 | 68 | 386 | 42 | 75 | 19 | 0.05 |
| L | 366 | 62 | 141 | 77 | 400 | 6 | 0.17 |
| acidophilus | 1329 | 17 | 89 | 51 | 112 | 78 | 0.01 |
| | 2715 | 24 | 102 | 69 | 289 | 113 | 0.01 |
| L. bulgaricus | 144 | 22 | 77 | 88 | 609 | 7 | 0.15 |
| | 381 | 12 | 69 | 78 | 313 | 32 | 0.03 |
| | 306 | 9 | 59 | 72 | 305 | 34 | 0.03 |
| L. paracasei | 186 | 70 | 106 | 53 | 506 | 3 | 0.38 |
| | 2112 | 47 | 138 | 90 | 337 | 45 | 0.02 |
| | 2145 | 43 | 153 | 92 | 427 | 50 | 0.02 |
| L. plantarums | 282 | 85 | 236 | 140 | 958 | 3 | 0.30 |
| | 5484 | 43 | 129 | 115 | 427 | 128 | 0.01 |
| | 3087 | 37 | 125 | 92 | 335 | 83 | 0.01 |

Purified NK cells from healthy donors were left untreated or treated with IL-2 (1000 units/ml) or the combination of anti-CD16mAb (3 μg/ml) and IL-2 (1000 units/ml) in the presence or absence of probiotic bacteria sAJ2 at 1:5 ratio (NK:sAJ2) for 18 hours. Afterwards, the levels of cytokines, growth factors and chemokines were determined using Bio-Plex Pro Human Cytokine 27-plex Array Kit. The ratio for IFN-g/IL-10 and IL-10/IFN-g were determined for each bacterial species.

TABLE 5
Production of cytokines, growth factors, and chemokines
by NK cells treated with bacterial strains

| | | Ratio of | | Ratio of | | Ratio of | | Ratio of |
|---|---|---|---|---|---|---|---|---|
| | G- | treated vs. | MCP- | treated vs. | IFN- | treated vs. | IL- | treated vs. |
| Bacteria | CSF | untreated | 1 | untreated | g-g | untreated | 10 | untreated |
| Control | 28 | | 258 | | 34 | | 6 | |
| | 54 | | 525 | | 99 | | 6 | |
| | 65 | | 305 | | 138 | | 8 | |
| S. | 149 | 5.3 | 502 | 1.9 | 785 | 23.1 | 63 | 10.5 |
| thermophilus | 87 | 1.6 | 169 | 0.3 | 1188 | 12.0 | 17 | 2.8 |
| | 101 | 1.6 | 332 | 1.1 | 2594 | 18.8 | 23 | 2.9 |
| B. breve | 363 | 13.0 | 265 | 1.0 | 99 | 2.9 | 127 | 21.2 |
| | 138 | 2.6 | 72 | 0.1 | 284 | 2.9 | 36 | 6.0 |
| | 289 | 4.4 | 153 | 0.5 | 844 | 6.1 | 68 | 8.5 |
| B. longum | 938 | 33.5 | 80 | 0.3 | 158 | 4.6 | 298 | 49.7 |
| | 244 | 4.5 | 33 | 0.1 | 671 | 6.8 | 52 | 8.7 |
| | 386 | 5.9 | 75 | 0.2 | 1282 | 9.3 | 68 | 8.5 |
| L. | 141 | 5.0 | 400 | 1.6 | 366 | 10.8 | 62 | 10.3 |
| acidophilus | 89 | 1.6 | 112 | 0.2 | 1329 | 13.4 | 17 | 2.8 |
| | 102 | 1.6 | 289 | 0.9 | 2715 | 19.7 | 24 | 3.0 |
| L. paracasei | 106 | 3.8 | 506 | 2.0 | 186 | 5.5 | 70 | 11.7 |
| | 138 | 2.6 | 337 | 0.6 | 2112 | 21.3 | 47 | 7.8 |
| | 153 | 2.4 | 427 | 1.4 | 2145 | 15.5 | 43 | 5.4 |
| L. plantarum | 236 | 8.4 | 958 | 3.7 | 282 | 8.3 | 85 | 14.2 |
| | 129 | 2.4 | 427 | 0.8 | 5484 | 55.4 | 43 | 7.2 |
| | 125 | 1.9 | 335 | 1.1 | 3087 | 22.4 | 37 | 4.6 |

Purified NK cells from healthy donors were left untreatred or treated with IL-2 (1000 units/ml) or the combination of anti-CD16mAb (3 μg/ml) and IL-2 (1000 units/ml) in the presence or absence of probiotic bacterial species as shown in the Table 1 at 1:5 ratio (NK:sonicated bacterial species) for 18 hours. Afterwards, the levels of cytokines, growth factors and chemokines were determined using Bio-Plex Pro Human Cytokine Array Kit.

TABLE 6
Production of Gro-alpha by NK cells treated with bacterial strains
Purified NK cells from healthy donors were left untreated or treated with
IL-2 (1000 units/ml) or the combination of anti-CD16mAb (3 μg/ml) and
IL-2 (1000 units/ml) in the presence or absence of probiotic bacterial
species at 1:5 ratio (NK:sonicated bacterial species) for 18 hours.
Afterwards, the levels of GRO were determined using Bio-Plex Pro
Human Cytokine Array Kit.

| | | | Ratio untreated |
|---|---|---|---|
| | Analyte | GRO pg/ml | vs. treated |
| Control | untreated | 48.26 | |
| | IL2 | 125.65 | |
| | IL2 + aCD16 | 230.74 | |
| L. plant | untreated | 159.96 | 3.3 |
| | IL2 | 200.15 | 1.6 |
| | IL2 + aCD16 | 208.92 | 0.9 |
| B. long | untreated | 333.91 | 6.9 |
| | IL2 | 280.33 | 2.2 |
| | IL2 + aCD16 | 230.74 | 1.0 |
| L. paracasei | untreated | 110.11 | 2.3 |
| | IL2 | 254.51 | 2.0 |
| | IL2 + aCD16 | 227.5 | 1.0 |
| B. infantis | untreated | 533.23 | 11.0 |
| | IL2 | 292.04 | 2.3 |
| | IL2 + aCD16 | 250.99 | 1.1 |
| L. casei | untreated | 208.92 | 4.3 |
| | IL2 | 272.74 | 2.2 |
| | IL2 + aCD16 | 218.04 | 0.9 |
| L. plant | untreated | 218.04 | 4.5 |
| | IL2 | | |
| | IL2 + aCD16 | 194.48 | 0.8 |
| L. gusseri | untreated | 183.6 | 3.8 |
| | IL2 | 221.15 | 1.8 |
| | IL2 + aCD16 | 178.36 | 0.8 |
| B. long | untreated | 265.32 | 5.5 |
| | IL2 | 250.99 | 2.0 |
| | IL2 + aCD16 | 250.99 | 1.1 |
| KE | untreated | 61.83 | 1.3 |
| | IL2 | 161.13 | 1.3 |
| | IL2 + aCD16 | 224.31 | 1.0 |
| S. thermo | untreated | 147.62 | 3.1 |
| | IL2 | 163.49 | 1.3 |
| | IL2 + aCD16 | 244.08 | 1.1 |

TABLE 7

| | | S. | B. | B. | L. | L. | L. |
|---|---|---|---|---|---|---|---|
| | | thermosphilus | breve | Longum | acidophilus | paracasei | plantarum |
| G-CSF | Untreated | 5.3 | 13.0 | 33.5 | 5.0 | 3.8 | 8.4 |
| | IL-2 | 1.6 | 2.6 | 4.5 | 1.6 | 2.6 | 2.4 |
| | IL-2 + anti- | 1.6 | 4.4 | 5.9 | 1.6 | 2.4 | 1.9 |
| | CD16 | | | | | | |
| IFN-g | Untreated | 23.1 | 2.9 | 4.6 | 10.8 | 5.5 | 8.3 |
| | IL-2 | 12.0 | 2.9 | 6.8 | 13.4 | 21.3 | 55.4 |
| | IL-2 + anti- | 18.8 | 6.1 | 9.3 | 19.7 | 15.5 | 22.4 |
| | CD16 | | | | | | |
| IL-10 | Untreated | 10.5 | 21.2 | 49.7 | 10.3 | 11.7 | 14.2 |
| | IL-2 | 2.8 | 6.0 | 8.7 | 2.8 | 7.8 | 7.2 |
| | IL-2 + anti- | 2.9 | 8.5 | 8.5 | 3.0 | 5.4 | 4.6 |
| | CD16 | | | | | | |
| MCP-1 | Untreated | 1.9 | 1.0 | 0.3 | 1.6 | 2.0 | 3.7 |
| | IL-2 | 0.3 | 0.1 | 0.1 | 0.2 | 0.6 | 0.8 |
| | IL-2 + anti- | 1.1 | 0.5 | 0.2 | 0.9 | 1.4 | 1.1 |
| | CD16 | | | | | | |

Higher secretion of IFN-g and MCP-1 by the combination of lactobacillus and Streptococcus species used in AJ4 formulation, thereas higher secretion of IL-10 and GCSF by bifidobacterial species used in AJ3 formuations were observed with NK cells were left untreated, or treated with IL-2 or IL-2 + anti-CD16mAb. The levels of increase were compared between bacterial strain treated NK samples with those of untreated samples for all three NK treatments (ratio of treated vs. untreated). Values in bold are for GCSF and IL-10, which were induced more by AJ3. Values in italics were induced more by AJ4.

TABLE 8

| | | L. | B. | L. | B. | L. |
|---|---|---|---|---|---|---|
| | | plantarum | Longum | paracasei | infantis | casei |
| GRO | Untreated | 3.3 | 6.9 | 2.3 | 11.0 | 4.3 |
| | IL-2 | 1.6 | 2.2 | 2.0 | 2.3 | 2.2 |
| | IL-2 + anti- | 0.9 | 1.0 | 1.0 | 1.1 | 0.9 |
| | CD16 | | | | | |

| | | L. | L. | B. | | S. |
|---|---|---|---|---|---|---|
| | | plantarum | gusseri | long | KE | thermo |
| GRO | Untreated | 4.5 | 3.8 | 5.5 | 1.3 | 3.1 |
| | IL-2 | | 1.8 | 2.0 | 1.3 | 1.3 |
| | IL-2 + anti- | 0.8 | 0.8 | 1.1 | 1.0 | 1.1 |
| | CD16 | | | | | |

Higher secretion of GRO by the bifidobacterial species used in AJ3 formulation in comparison to lactobacillus or Streptococcus species was observed when NK cells were left untreated, or treated with IL-2 or IL-2 + anti-CD16mAb. The levels of increase were compared between bacterial strain treated NK samples with those of non bacterial strain treated samples for all three NK treatments (ratio of treated vs. untreated). Values in bold for GRO were induced more by AJ3. Values in italics were induced less by AJ4.

TABLE 9
Exp285 Bac testing

| PBMC | | | | NK | | | |
|---|---|---|---|---|---|---|---|
| IFN-γ | CTRL | AJ3 | AJ4 | IFN-γ | CTRL | AJ3 | AJ4 |
| untreated | 5.73 | 351.91 | 4989.00 | untreated | 9.05 | 14.43 | 12.59 |
| IL-2 treated | 95.13 | 28830.00 | 59597.00 | IL-2 treated | 32.85 | 1917.00 | 13347.00 |
| IL-2 + aCD16 | 2311.00 | | | IL-2 + aCD16 | 492.03 | | |
| treated | | | | treated | | | |

| PBMC | | | | NK | | | |
|---|---|---|---|---|---|---|---|
| TNF-α | CTRL | AJ3 | AJ4 | TNF-α | CTRL | AJ3 | AJ4 |
| untreated | 6.55 | 5232.00 | 13653.00 | untreated | 11.89 | 1541.00 | 1084.00 |
| IL-2 treated | 37.17 | 6674.00 | 19285.00 | IL-2 treated | 63.60 | 1496.00 | 1973.00 |
| IL-2 + aCD16 | 533.23 | | | IL-2 + aCD16 | 214.96 | | |
| treated | | | | treated | | | |

| PBMC | | | | NK | | | |
|---|---|---|---|---|---|---|---|
| IL-6 | CTRL | AJ3 | AJ4 | IL-6 | CTRL | AJ3 | AJ4 |
| untreated | 2.11 | 12622.00 | 8654.00 | untreated | 4.20 | 3786.00 | 1149.00 |
| IL-2 treated | 12.50 | 12062.00 | 12541.00 | IL-2 treated | 13.53 | 3406.00 | 1945.00 |
| IL-2 + aCD16 | 744.33 | | | IL-2 + aCD16 | 5.35 | | |
| treated | | | | treated | | | |
| PBMC | | | | NK | | | |
| IL-10 | CTRL | AJ3 | AJ4 | IL-10 | CTRL | AJ3 | AJ4 |
| untreated | 7.48 | 26385.00 | 4754.00 | untreated | 8.29 | 4871.00 | 479.17 |
| IL-2 treated | 16.88 | 22515.00 | 10066.00 | IL-2 treated | 14.68 | 2736.00 | 351.91 |
| IL-2 + aCD16 | 52.36 | | | IL-2 + aCD16 | 8.70 | | |
| treated | | | | treated | | | |

| PBMCs | | Untreated | AJ3 | AJ4 | NK | Untreated | AJ3 | |
|---|---|---|---|---|---|---|---|---|
| IFN-g/IL- | Untreated | 0.8 | 0.013 | 1.1 | | 1.08 | 0.003 | 0.03 |
| 10 ratio | | | | | | | | |
| | IL-2 | 5.6 | 1.28 | 5.9 | | 2.2 | 0.7 | 38 |
| | treated | | | | | | | |

Higher secretion of IFN-g and TNF-a in comparison to IL-6 and IL-10 by AJ4 treatment, in comparison to AJ3 treatment in PBMCs and NK cells. Higher IL-6 and IL-10 in comparison to IFN-g and TNF-a in PBMCs and NK cells when the immune cells were treated with AJ3. PBMCs and NK cells at (1 × 10⁶ cells/ml) were left untreated, or treated with IL-2 (1000 u/ml). The ratio of secretion were determined between IFN-g and IL-10 in all the samples. Values in bold are for IL-10, which was induced more by AJ3. Values in italics are for IFN-g, which was induced more by AJ4.

TABLE 10
Production of IFN-γ and IL-10 by NK cells treated with bacterial strains

| | | |
|---|---|---|
| NK alone | NK + auto monocyte | monocyte alone |

| | | | | | | | | |
|---|---|---|---|---|---|---|---|---|
| | | IL-2 + | | | IL-2 + | | | IL-2 + |
| untreated | IL-2 | αCD16 | untreated | IL-2 | αCD16 | untreated | IL-2 | αCD16 |

IFN-γ (TC)

| | | | | | | | | | |
|---|---|---|---|---|---|---|---|---|---|
| CTRL | 0.95 | 1.20 | 1.45 | 1.20 | 1.26 | 11.69 | 1.14 | 1.08 | 1.08 |
| AJ3 | 1.20 | 2.59 | 10.19 | 1.20 | 36.19 | 449.15 | 0.95 | 4.51 | 3.10 |
| AJ4 | 1.20 | 10.76 | 18.75 | 5.98 | 412.11 | 1335.00 | 9.65 | 66.78 | 74.12 |

IL-10 (TC)

| | | | | | | | | | |
|---|---|---|---|---|---|---|---|---|---|
| CTRL | 1.83 | 2.02 | 1.83 | 2.20 | 2.57 | 4.18 | 2.02 | 1.83 | 1.83 |
| AJ3 | 3.82 | 2.39 | 2.93 | 19.88 | 28.26 | 61.65 | 25.50 | 27.85 | 5.22 |
| AJ4 | 2.20 | 2.57 | 2.20 | 28.67 | 26.26 | 48.14 | 15.53 | 17.43 | 12.37 |

| | | IL-2 | IL-2 + anti-CD16 |
|---|---|---|---|
| Ratio of IFN-g/IL-10 | Untreated | 0.48 | 3 |
| | AJ3 treated | 1.29 | 7 |
| | AJ4 treated | 16 | 28 |

Higher ratio of IFN-g secretion of IL-10 secretion by AJ4 treatment in comparison to AJ3 treatment in NK cells at (1 × 10⁶ cells/ml) cultured in the presence and absence of monocytes at 1 × 10⁶ cells/ml) were seen when NK cells were left untreated of treated with IL-2 (1000 units/ml) or the combination of anti-CD16mAb (3 μg/ml) and IL-2 (1000 units/ml). Values in bold are for IL-10, which was induced more by AJ3. Values in italics are for IFN-g, which was induced more by AJ4.

### Example 4: Materials and Methods for Examples 5-8

### Cell Lines, Reagents, and Antibodies

RPMI 1640 (Life Technologies, CA, USA) supplemented with 10% fetal bovine serum (FBS) (Gemini Bio-Products, CA) was used to culture peripheral blood mononuclear cells (PBMCs), NK cells, and CD8+ T cells. Recombinant IL-2 was obtained from NIH-BRB. Anti-CD16 mAbs, and anti-CD3/CD28 mAbs were obtained from Biolegend (San Diego, CA). Oral squamous carcinoma stem cells (OSCSCs) were isolated from patients with tongue tumors at UCLA. Human ELISA kits for IFN-γ were purchased from Biolegend (San Diego, CA). Chroimum-51 radionucleotide was purchased from PerkinElmer, CA, USA.

### Isolation of Human PBMCs, NK Cells, and CD8+ T Cells

Written informed consents approved by UCLA Institutional Review Board (IRB) were obtained from healthy individuals, and all procedures were approved by the UCLA-IRB. PBMCs were isolated from peripheral blood as described before. PBMCs were used to isolate NK cells, and CD8+ T cells using the EasySep® Human NK cell and Easy Sep® Human CD8+ T cells, respectively, purchased from stem cell technologies (Vancouver, BC, Canada). Isolated NK cells and CD8+ T cells were stained with anti-CD16, anti-CD3/CD8 antibodies, respectively, to measure the cell purity using flow cytometric analysis.

### Enzyme-Linked Immunosorbent Assays (ELISAs), Enzyme-Linked Immunospot (ELISpot) and Multiplex Cytokine Arrays

Single ELISAs were performed as previously described. To analyze and obtain the cytokine and chemokine concentrations, a standard curve was generated by either two- or three-fold dilution of recombinant cytokines provided by the manufacturer. The ELISpot was conducted according to manufacturer's instructions. The number of IFN-γ secreting cells was determined by using human IFN-γ single-color enzymatic ELISpot assay, and analyzed by the ImmunoSpot® S6 UNIVERSAL analyzer and ImmunoSpot® software (all CTL Europe GmbH, Bohn, Germany). For multiple cytokine arrays, the levels of cytokines and chemokines were also determined by multiplex cytokine arrays as recommended by the manufacturer. Analysis was performed using a luminex instrument (MAGPIX, Millipore, Billerica, MA), and data were analyzed using the proprietary software (xPONENT 4.2, Millipore, Billerica, MA).

### ⁵¹Cr Release Cytotoxicity Assay

The ⁵¹Cr release assay was performed as described previously. Briefly, different numbers of effector cells were incubated with ⁵¹Cr-labeled target cells. After a 4-hour incubation period, the supernatants were harvested from each sample and the released radioactivity was counted using the gamma counter. The percentage specific cytotoxicity was calculated as follows:

$$\% Cytotoxicity = \frac{Experimental cpm - spontaneous cpm}{Total cpm - spontaneous cpm}$$

Lytic units (LU) 30/10⁶ is calculated by using the inverse of the number of effector cells needed to lyse 30% of tumor target cells×100.

### Sonication of Probiotic Bacteria AJ2, AJ3 and AJ4

Gram-positive probiotic bacteria strains for AJ2, AJ3, and AJ4 were weighed and re-suspended in RPMI 1640 medium containing 10% FBS at a concentration of 10 mg/ml. The bacteria were thoroughly vortexed and sonicated on ice for 15 seconds at 6 to 8 amplitudes. Sonicated samples were then incubated for 30 seconds on ice, and the cycle was repeated for five rounds. After every five rounds of sonication, the samples were examined under the microscope until at least 80% of bacterial walls were lysed. It was determined that approximately 20 rounds of sonication/incubation on ice were necessary to achieve complete sonication. Finally, the sonicated probiotic bacteria, sAJ2, sAJ3, and sAJ4 were aliquoted and stored at −80° C. until use. Sonication of bacteria is not required or necessary to render its activities presented herein.

### Statistical Analysis

All statistical analyses were performed using the GraphPad Prism-8 software. An unpaired or paired, two-tailed student's t-test was performed for the statistical analysis for experiments with two groups. One-way ANOVA with a Bonferroni post-test was used to compare different groups for experiments with more than two groups. Duplicate or triplicate samples were used in the studies. (n) denotes the number of healthy individuals for each experimental condition. The following symbols represent the levels of statistical significance within each analysis: ***(p value <0.001), **(p value 0.001-0.01), *(p value 0.01-0.05).

### Example 5: Decreased IFN-γ and Increased IL-10 Secretions by sAJ3 Treated PBMCs in Comparison to sAJ4 and sAJ2 Treated PBMCs

Examples 5-8 demonstrate how the combination of different strains, SAJ2, sAJ3, and sAJ4 differ in their potential to activate PBMCs, NK cells and CD8+ T cells. In addition, we compared the functional activation of NK cells by sAJ3 and sAJ4 in the presence of monocytes.

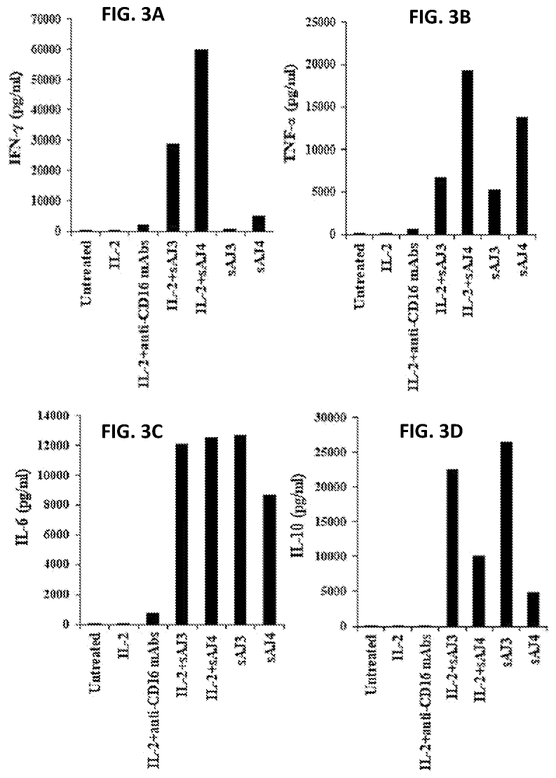

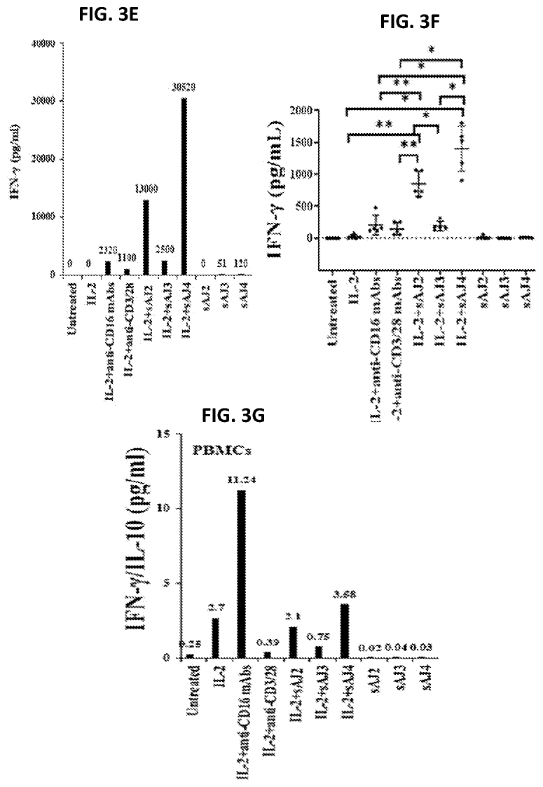

We observed increased secretion of IFN-γ (FIGS. 3A, 3E, and 3F, and Table 11), TNF-α (FIG. 3B), and IL-6 (FIG. 3C) in PBMCs when treated with IL-2 and sonicated AJ4 (sAJ4) in comparison to other treatments as shown in figures. Decreased IL-10 secretion was seen in sAJ4 treated PBMCs in comparison to sAJ3 treated PBMCs (FIG. 3D). CD16 receptor plays a significant role in increased secretion of IFN-γ in the presence of IL-2 treatment. Since IL-2 or the combination of IL-2 and CD16 cross-linking increased IFN-γ secretion in the presence of negligible IL-10 secretion, the ratio of IFN-γ to IL-10 remained higher in these treatments, with the highest seen in IL-2 and anti-CD16 mAbs treated PBMCs (FIG. 3G and Table 12A). Ratio of IFN-γ to IL-10 was much higher in IL-2+sAJ4 treated PBMCs in comparison to IL-2+sAJ2 or IL-2+sAJ3 treated PBMCs, IL-2+sAJ3 having the lowest ratio (FIG. 3G and Table 12B). Even though sAJ4 alone was able to trigger some IFN-γ secretion, the synergistic effect was seen when PBMCs were treated with IL-2 and sAJ4. The direct effect of SAJ4 without IL-2 treatment was higher than sAJ3 or sAJ2 on PBMCs (FIGS. 3A, 3E and 3F). Of interest, is the much lower ratio of IFN-γ to IL-10 in IL-2+anti-CD3/CD28 mAbs treated PBMCs, when either compared to IL-2 alone or IL-2+anti-CD16mAbs (FIG. 3G).

### Example 6: Decreased IFN-γ and Increased IL-10 Secretion by sAJ3 Treated NK Cells in Comparison to sAJ4 and sAJ2 Treated NK Cells

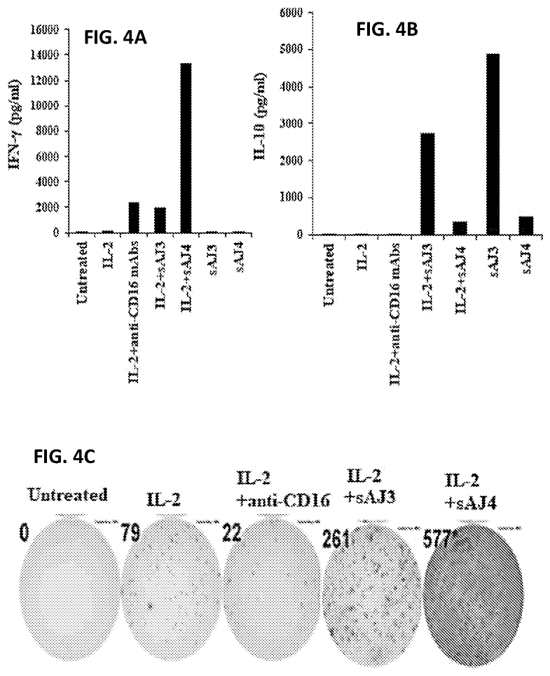

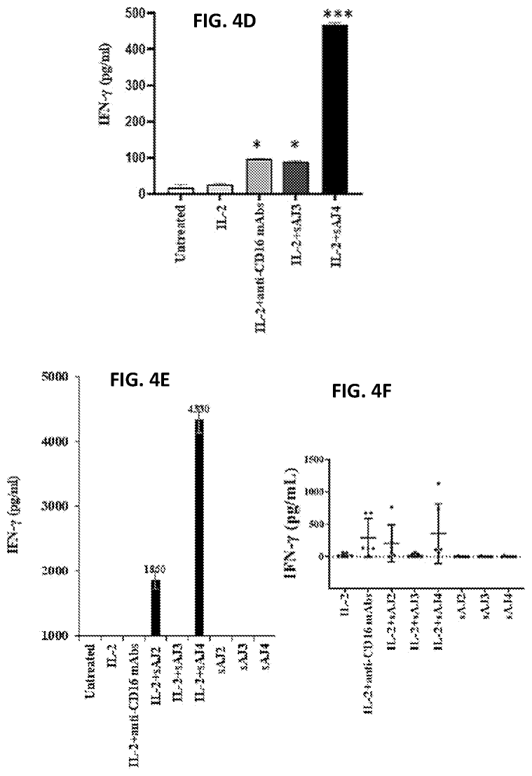

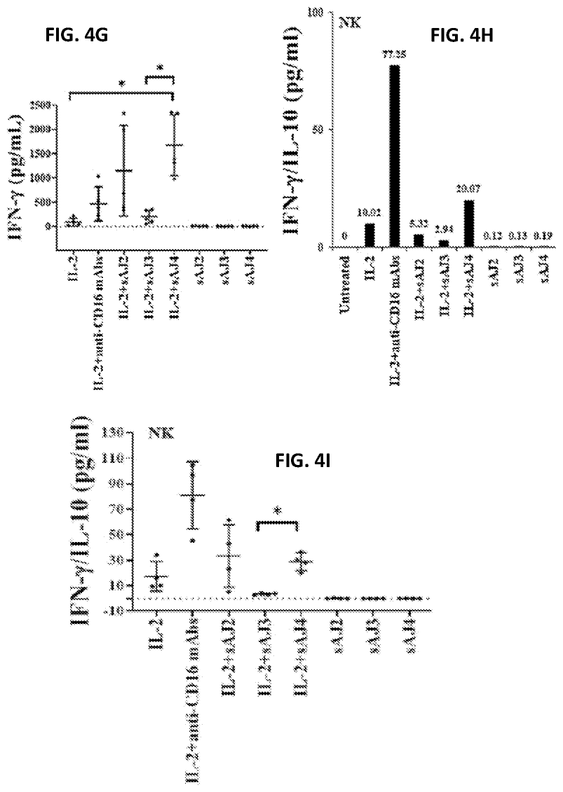

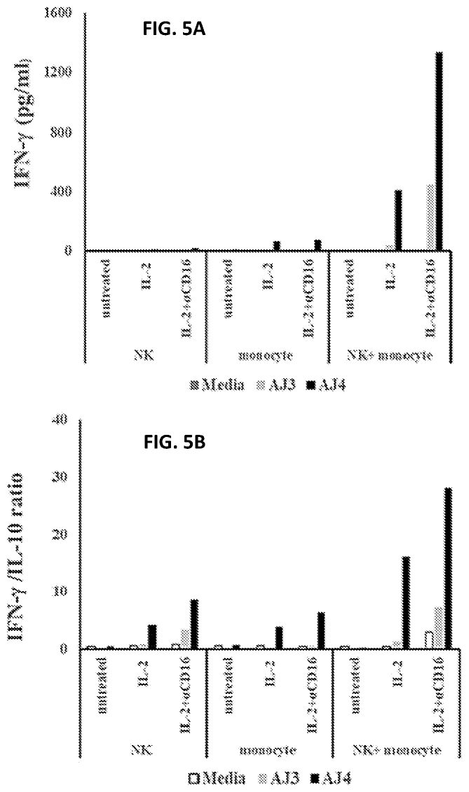

NK cells were sorted from the PBMCs and used in the treatments as described above for PBMCs. Similar to PBMCs, we observed significantly increased levels of IFN-γ in NK cells when treated with IL-2 and sAJ4 in comparison to IL-2+sAJ3 or IL-2+sAJ2 as shown in the figures (FIGS. 4A, 4C, 4D, 4E, 4F, and 4G). The ratio of IFN-γ to IL-10 was the highest in IL-2+sAJ4 and the lowest in IL-2+sAJ3 (FIGS. 4H and 4I, and Table 13). As mentioned above, the combination of IL-2 and CD16 cross-linking increased IFN-γ secretion, and the ratio of IFN-γ to IL-10 was seen the highest in IL-2 and anti-CD16 mAbs treated NK cells (FIGS. 4H and 4I).

### Example 7: Decreased IFN-γ Secretion in SAJ3 Treated NK Cells Cultured with Monocytes in Comparison to sAJ4 Treated Cells

To assess the effect of probiotics on IFN-γ and IL-10 secretion by the NK cells cultured with autologous monocytes, we treated NK and monocytes co-cultures either with IL-2 alone or IL-2 with sAJ3 or sAJ4, or sAJ3 or sAJ4 alone and determined IFN-γ and IL-10 secretion (FIG. 5). Both in the absence or presence of monocytes, NK cells exhibited higher IFN-γ secretion when treated with sAJ4 in comparison to sAJ3 (FIG. 5A). Also, in the absence or presence of monocytes, NK cells exhibited higher IFN-γ to IL-10 ratio when treated with sAJ4 in comparison to sAJ3 (FIG. 5B). Even though sAJ4 alone was able to trigger some IFN-γ secretion, the synergistic effect was seen when NK cells were treated with IL-2 and sAJ4. The direct effect of SAJ4 without IL-2 treatment was higher than sAJ3 by NK cells (FIG. 5)

### Example 8: Unlike NK Cells CD8+ T Cells Secrete Considerably Lower IFN-γ when Treated with IL-2 and Probiotic Bacteria

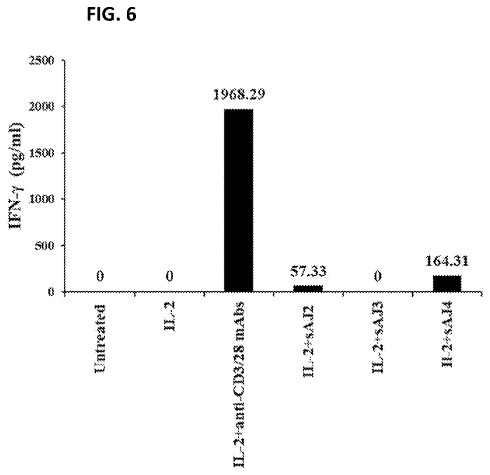

CD8+ T cells were sorted from the peripheral blood and treated with IL-2 or IL-2+anti-CD3/CD28 mAbS or IL-2 with each of SAJ4 and sAJ3. Treatment with IL-2+anti-CD3/CD28 mAbs induced the highest secretion of IFN-γ, whereas combination of IL-2+sAJ4 was considerably lower, but still higher than IL2+sAJ3 in comparison to IL-2+anti-CD3/CD28 mAbs (FIG. 6).

### Discussion

In the past several years, there has been a significant interest in the role of microbiome and probiotics in contributing to the health and well-being of humans. Their role in alleviating disease symptoms and prevention has been shown in many disease models, however, our understanding of the mechanisms by which they exert their effect is still in its infancy. Here, we demonstrated disease specific functions of three different formulations of probiotics in auto-immunity, cancer, and disease prevention with emphasis on alleviating ALS symptoms and using them as adjunct to other treatments to significantly delay disease progression. NK-CLK (AJ2), AI-Pro (AJ3) and CA/I-Pro (AJ4) are the three sets of formulations with distinct functions. Since the emphasis is on ALS, we delineated the differences in function of this probiotic formulation in comparison to the other two probiotics. AJ3 probiotic was formulated to augment anti-inflammatory cytokine to counter the aggressive nature of pro-inflammatory cytokine such as IFN-g which is primarily secreted by NK cells and T cells. ALS patients have significantly higher functions of NK and CD8+ T cells and they secrete large amounts of IFN-γ upon activation. Indeed, the serum levels of IFN-γ in patients is higher in comparison to the healthy controls, and even upon treatment with NAC which blocks most of the other pro-inflammatory cytokines secreted from the immune cells, it is not capable of decreasing IFN-γ and TNF-α and IL-17a. To counter this effect, we pursued the formulation of a probiotic bacteria which is capable of secreting very high levels of anti-inflammatory cytokine IL-10 to counter the function of IFN-γ. As can be observed from the data presented herein, significant increase in IL-10 secretion can be seen by sAJ3 probiotic bacteria when PBMCs, NK cells, and combination of NK cells with monocytes were tested and the results were compared to sAJ2 and sAJ4. It is of significance to note that dynamics of IFN-γ, TNF-α, IL-6, and IL-10 secretion are quite different when PBMCs are triggered by sAJ3 and sAJ4 as shown in the present studies (FIGS. 3 and 4). Even though there are significant secretion of TNF-α, IL-6 and IL-10 in the presence of both sAJ3 and sAJ4, IFN-γ secretion mainly occurs when cells are activated with IL-2 or other activators such as anti-CD16 mAbs (FIGS. 3 and 4). Indeed, IL-2+anti-CD16 mAbs activated PBMCs and NK cells resulted in a much greater increase in IFN-γ/IL-10 ratio, indicating that NK cells when receive activation signals through CD16 receptor significantly augment the levels of IFN-γ in the presence of no or low levels of IL-10 induction (FIGS. 3G, 4H and 4I). sAJ4, unlike sAJ3, synergistically augment IFN-γ in the presence of decreased induction of IL-10. However, sAJ3 acts on the opposite, increasing IL-10 in the presence of less IFN-γ, a scenario which is desirable in auto-immune diseases such as ALS. Of interest, is the observation that T cells when activated through IL-2 and anti-CD3/CD28 mAbs secrete higher IL-10 and the ratio of IFN-γ to IL-10 is much lower than that observed when NK cells are activated through IL-2 and anti-CD16 mAbs. However, when T cells are triggered by the sAJ2, sAJ3 and sAJ4 they secrete relatively much less IFN-γ in comparison to NK cells which significant levels of IFN-γ is released, and the levels exceed those triggered by IL-2+anti-CD16 mAbs. Whereas, the levels of IL-2 and probiotic in CD8+ T cells is much less than trigger by IL-2+anti-CD3/CD28 mAbs.

The observation seen in the presence of SAJ3 and sAJ4 on NK cells and synergy with IL-2 and IL-2+anti-CD16 mAbs is surprising and unexpected, and has significant implications for the role of probiotic bacteria in prevention and treatment of disease. If there are no local activation (by e.g., infection, disease) which may not trigger secretion of IL-2 or cross linking of important receptors on immune cells such as NK cells, the default of the effect is increase in anti-inflammatory cytokines, such as those seen in the presence of activation of PBMCs and NK cells by the bacteria alone. Higher induction of IL-10 in this respect with bacteria should provide an anti-inflammatory environment over pro-inflammatory environment, since increases in IL-10 and IL-6 is evident in the presence of no or very slight induction of IFN-γ secretion. The secretion of TNF-α has a similar trend to IFN-γ in which there is higher induction of TNF-α in the presence of SAJ4 and lower in the presence of SAJ3. Whereas the release of IL-6 and IL-10 is much higher by sAJ3 than sAJ4. On the other hand, where there may be local activation of immune cells and secretion of IL-2, probiotic bacteria will sway the pendulum towards a pro-inflammatory microenvironment where IFN-γ will be highly increased. Such finetuning of the environment is elegant and effective since such environment will not only be localized to where the significant aid is needed, but also will ensure that the individual will mount an effective immunity in areas where it is needed.

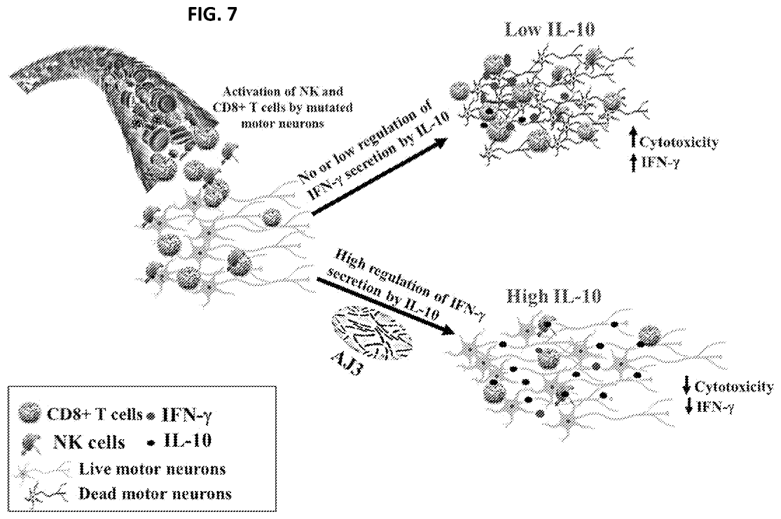

In conclusion presented herein are three different formulations of probiotic bacteria with different effect on the activation of PBMCs, NK, and CD8+ T cells. AJ3 is effective in increasing IL-10 and regulating the levels and function of IFN-γ, whereas AJ4 triggers higher levels of IFN-γ without the increase in IL-10 and therefore, this probiotic will be effective in the treatment of cancer and infections where the increased levels and functions of IFN-γ is required for differentiation of tumor cells and prevention of the replication of virus, respectively. On the other hand, AJ3 will be effective in alleviating auto-immunity, in particular in ALS since it will greatly regulate the levels and function of IFN-γ, decreasing over activation and death of motor neurons (FIG. 7). AJ2 on the other hand can be used in healthy individuals as a maintenance since it produces IFN-γ in the presence of intermediate levels of IL-10, to regulate the IFN-γ, and therefore, a balance between the secretion of IFN-γ and IL-10 is established.

### Example 9: Anti-Cancer Activity of AJ4 In Vivo

Analysis of human pancreatic cancer cell growth in humanized-BLT mice. Humanized-BLT (hu-BLT; human bone marrow/liver/thymus) mice were generated as previously described (Kaur et al., (2020) (′ancers 12:63; Kaur et al., 2018) Oncoimmunology 7 (5): e1426518, each of which is incorporated herein by reference). In vivo growth of pancreatic tumors was performed by orthotopic tumor implantation in the pancreas of hu-BLT mice. To establish orthotopic tumors, mice were anesthetized using isoflurane, and 1×10⁶ tumors in a mixture with Matrigel (10 μL) (Corning, NY, USA) were injected in the pancreas using insulin syringe. Mice received 1-1.5×10⁶ super-charged NK cells via tail vein injection 7 to 10 days after the tumor implantation. They were also fed AJ4 (2-5 billion/dose) orally. The first dose of AJ4 was given one or two weeks before tumor implantation, and feeding was continued throughout the experiment at an interval of every 48 h. Mice were euthanized when signs of morbidity were evident. Pancreas, pancreatic tumors, bone marrow, spleen, and peripheral blood were harvested and single cell suspensions were prepared from each tissue as described previously. The groups are as follows; BLT control, BLT fed with AJ4 alone, BLT injected with IL-2 alone, BLT implanted with only tumor, BLT fed with AJ4 and implanted with tumor, BLT injected with IL-2 and implanted with tumor, BLT fed with AJ4 and implanted with tumor and injected with IL-2, BLT implanted with tumor and injected with IL-2. 3 mice per group were used.

TABLE 11
Increased IFN-γ by sAJ4 treated PBMCs
in comparison to sAJ3 and sAJ2 treated PBMCs.

| Treatments | Donor 1 | Donor 2 | Donor 3 | Donor 4 | Donor 5 |
|---|---|---|---|---|---|
| Untreated | 2.11 | 16.79 | 1.67 | 2.04 | 11.27 |
| IL-2 | 41.67 | 654.21 | 622.12 | 1356 | 50.96 |
| IL-2 + anti-CD16 | 172.54 | 4802 | 591.61 | 357.77 | 164.07 |
| mAbs | | | | | |
| IL-2 + anti-CD3/28 | 190.8 | 405.71 | 471.78 | 80.13 | 24.82 |
| mAbs | | | | | |
| IL-2 + sAJ2 | 121.33 | 5265 | NA | 780.13 | 46.66 |
| IL-2 + sAJ3 | 37.68 | 2724 | 654.21 | 205.75 | 28.85 |
| IL-2 + sAJ4 | 195.66 | 6653 | 1764 | 989.1 | 54.26 |
| sAJ2 | 14.72 | NA | 395.63 | 73.38 | 14.3 |
| sAJ3 | 16.07 | 20.02 | NA | 27.72 | 15.84 |
| sAJ4 | 23.79 | 186.06 | NA | 416.04 | 23.13 |

PBMCs were isolated from healthy individuals' peripheral blood as described in Materials and Methods section in Example 4. PBMCs were left untreated or treated with IL-2 (1000 U/ml) or with a combination of IL-2 (1000 U/ml) and anti-CD16 mAbs (3 μg/ml) or with a combination of IL-2 (1000 U/ml) and anti-CD3/28 antibody (25 μl/ml) or with a combination of IL-2 (1000 U/ml) and sAJ2 (PBMC: sAJ2, 1:20) or with a combination of IL-2 (1000 U/ml) and sAJ3 (PBMC: sAJ3, 1:20) or with a combination of IL-2 (1000 U/ml) and sAJ4 (PBMC: sAJ4, 1:20) for 18 hours before the supernatants were harvested from PBMCs to determine IFN-γ secretion using multiplex assay.

Tables 12A-12B Increased IFN-γ and decreased IL-10 secretions by sAJ4 treated PBMCs in comparison to sAJ3 and sAJ2 treated PBMCs.

TABLE 12A

| IFN-γ/IL-10 | | | | | | |
|---|---|---|---|---|---|---|
| (pg/ml) | Donor | Donor | Donor | Donor | Donor | Donor |
| PBMCs | 1 | 2 | 3 | 4 | 5 | 6 |
| Untreated | 0.25 | 0.22 | 0.23 | 0.29 | 0.55 | 0.14 |
| IL-2 | 2.7 | 1.66 | 26.94 | 61.01 | 11.04 | 27.61 |
| IL-2 + anti- | 11.24 | 12.65 | 51.33 | 54.29 | 66.16 | 24.18 |
| CD16 mAbs | | | | | | |
| IL-2 + anti- | 0.39 | 0.51 | 1.16 | 2.64 | 0.11 | 0.13 |
| 28 mAbsCD3/ | | | | | | |

PBMCs were isolated from healthy individuals' peripheral blood as described in Materials and Methods section in Example 4. PBMCs were left untreated or treated with IL-2 (1000 U/ml) or with a combination of IL-2 (1000 U/ml) and anti-CD16 mAbs (3 μg/ml) or with a combination of IL-2 (1000 U/ml) and anti-CD3/28 antibody (25 μl/ml) for 18 hours before the supernatants were harvested from PBMCs to determine IFN-γ and IL-10 secretion using specific single ELISAs, and ratio of IFN-γ to IL-10 was determined (Table 12A).

TABLE 12B

| IFN-γ/IL-10 (pg/ml) PBMCs | Media | sAJ3 | sAJ4 |
|---|---|---|---|
| Untreated | 0.8 | 0.013 | 1.1 |
| IL-2 | 5.6 | 1.28 | 5.9 |

PBMCs were left untreated or treated with IL-2 (1000 U/ml) or with sAJ3 (PBMC: sAJ3, 1:20) or with sAJ4 (PBMC: sAJ4, 1:20) or with a combination of IL-2 (1000 U/ml) and SAJ3 (PBMC: sAJ3, 1:20) or with a combination of IL-2 (1000 U/ml) and sAJ4 (PBMC: sAJ4, 1:20) for 18 hours before the supernatants were harvested from PBMCs to determine IFN-γ and IL-10 secretion using multiplex assay, and ratio of IFN-γ to IL-10 was determined (Table 12B).
Tables 13A-13B Increased IFN-γ and decreased IL-10 secretions by sAJ4 treated NK cells in comparison to sAJ3 and sAJ2 treated NK cells.

TABLE 13A

| IFN-γ/IL-10 (pg/ml) NK | Donor 1 | Donor 2 | Donor 3 | Donor 4 | Donor 5 |
|---|---|---|---|---|---|
| IL-2 | 34.07 | 10.02 | 16.1 | 0.18 | 9.45 |
| IL-2 + anti-CD16 mAbs | 104.9 | 77.25 | 45.51 | 9.66 | 96.94 |
| IL-2 + sAJ3 | 3.93 | 2.94 | 3.32 | 0.03 | 4.32 |
| IL-2 + sAJ4 | 30.58 | 20.07 | 36.62 | 0.06 | 28.12 |

NK cells were isolated from healthy individuals' PBMCs as described in Materials and Methods section in Example 4. NK cells were treated with IL-2 (1000 U/ml) or with a combination of IL-2 (1000 U/ml) and anti-CD16 mAbs (3 μg/ml) or with a combination of IL-2 (1000 U/ml) and sAJ3 (NK: sAJ3, 1:20) or with a combination of IL-2 (1000 U/ml) and sAJ4 (NK: sAJ4, 1:20) 18 hours before the supernatants were harvested from PBMCs to determine IFN-γ and IL-10 secretion using specific single ELISAs, and ratio of IFN-γ to IL-10 was determined (Table 13A).

TABLE 13B

| IFN-γ/IL-10 (pg/ml) NK | Media | sAJ3 | sAJ4 |
|---|---|---|---|
| Untreated | 1.08 | 0.003 | 0.03 |
| IL-2 | 2.2 | 0.7 | 38 |

NK cells were left untreated or treated with IL-2 (1000 U/ml) or with sAJ3 (NK: sAJ3, 1:20) or with sAJ4 (NK: sAJ4, 1:20) or with a combination of IL-2 (1000 U/ml) and sAJ3 (NK: sAJ3, 1:20) or with a combination of IL-2 (1000 U/ml) and sAJ4 (NK: sAJ4, 1:20) for 18 hours before the supernatants were harvested from PBMCs to determine IFN-γ and IL-10 secretion using multiplex assay, and ratio of IFN-γ to IL-10 was determined (Table 13B).

### REFERENCES

1. Vivier E, Raulet D H, Moretta A, Caligiuri M A, Zitvogel L, Lanier L L, et al. Innate or adaptive immunity? The example of natural killer cells. Science (New York, N Y). 2011; 331 (6013): 44-9.
2. Cooper M A, Fehniger T A, Caligiuri M A. The biology of human natural killer-cell subsets. Trends in immunology. 2001; 22 (11): 633-40.
3. Sun J C, Lanier L L. Is There Natural Killer Cell Memory and Can It Be Harnessed by Vaccination? NK Cell Memory and Immunization Strategies against Infectious Diseases and Cancer. Cold Spring Harb Perspect Biol. 2018; 10 (10).
4. Shaw S Y, Tran K, Castoreno A B, Peloquin J M, Lassen K G, Khor B, et al. Selective modulation of autophagy, innate immunity, and adaptive immunity by small molecules. ACS chemical biology. 2013; 8 (12): 2724-33.
5. Pant H, Hughes A, Miljkovic D, Schembri M, Wormald P, Macardle P, et al. Accumulation of effector memory CD8+ T cells in nasal polyps. American journal of rhinology & allergy. 2013; 27 (5): e117-26.
6. Tomala J, Chmelova H, Mrkvan T, Rihova B, Kovar M. In vivo expansion of activated naive CD8+ T cells and NK cells driven by complexes of IL-2 and anti-IL-2 monoclonal antibody as novel approach of cancer immunotherapy. J Immunol. 2009; 183 (8): 4904-12.
7. Tanaka J, Toubai T, Miura Y, Tsutsumi Y, Kato N, Umehara S, et al. Differential expression of natural killer cell receptors (CD94/NKG2A) on T cells by the stimulation of G-CSF-mobilized peripheral blood mononuclear cells with anti-CD3 monoclonal antibody and cytokines: a study in stem cell donors. Transplantation proceedings. 2004; 36 (8): 2511-2.
8. Burke S, Lakshmikanth T, Colucci F, Carbone E. New views on natural killer cell-based immunotherapy for melanoma treatment. Trends in immunology. 2010; 31 (9): 339-45.
9. Larsen S K, Gao Y, Basse P H. NK cells in the tumor microenvironment. Critical reviews in oncogenesis. 2014; 19 (1-2): 91-105.
10. Nolibe D, Poupon M F. Enhancement of pulmonary metastases induced by decreased lung natural killer cell activity. Journal of the National Cancer Institute. 1986; 77 (1): 99-103.
11. Imai K, Matsuyama S, Miyake S, Suga K, Nakachi K. Natural cytotoxic activity of peripheral-blood lymphocytes and cancer incidence: an 11-year follow-up study of a general population. Lancet (London, England). 2000; 356 (9244): 1795-9.
12. Harning R, Koo G C, Szalay J. Regulation of the metastasis of murine ocular melanoma by natural killer cells. Investigative ophthalmology & visual science. 1989; 30 (9): 1909-15.
13. Coca S, Perez-Piqueras J, Martinez D, Colmenarejo A, Saez M A, Vallejo C, et al. The prognostic significance of intratumoral natural killer cells in patients with colorectal carcinoma. Cancer. 1997; 79 (12): 2320-8.
14. Zanetti M. Tapping CD4 T cells for cancer immunotherapy: the choice of personalized genomics. J Immunol. 2015; 194 (5): 2049-56.
15. Gerosa F, Baldani-Guerra B, Nisii C, Marchesini V, Carra G, Trinchieri G. Reciprocal activating interaction between natural killer cells and dendritic cells. J Exp Med. 2002; 195 (3): 327-33.
16. Assarsson E, Kambayashi T, Schatzle J D, Cramer S O, von Bonin A, Jensen P E, et al. NK cells stimulate proliferation of T and NK cells through 2B4/CD48 interactions. J Immunol. 2004; 173 (1): 174-80.
17. Zingoni A, Sornasse T, Cocks B G, Tanaka Y, Santoni A, Lanier L L. Cross-talk between activated human NK cells and CD4+ T cells via OX40-OX40 ligand interactions. J Immunol. 2004; 173 (6): 3716-24.
18. Crouse J, Xu H C, Lang P A, Oxenius A. NK cells regulating T cell responses: mechanisms and outcome. Trends Immunol. 2015; 36 (1): 49-58.
19. Crome S Q, Lang P A, Lang K S, Ohashi P S. Natural killer cells regulate diverse T cell responses. Trends Immunol. 2013; 34 (7): 342-9.
20. Zingoni A, Ardolino M, Santoni A, Cerboni C. NKG2D and DNAM-1 activating receptors and their ligands in NK-T cell interactions: role in the NK cell-mediated negative regulation of T cell responses. Front Immunol. 2012; 3:408.
21. Pallmer K, Oxenius A. Recognition and Regulation of T Cells by NK Cells. Front Immunol. 2016; 7:251.
22. Nielsen N, Ødum N, Ursø B, Lanier L L, Spee P. Cytotoxicity of CD56 (bright) NK cells towards autologous activated CD4+ T cells is mediated through NKG2D, LFA-1 and TRAIL and dampened via CD94/NKG2A. PLOS One. 2012; 7 (2): e31959-e.
23. Noval Rivas M, Hazzan M, Weatherly K, Gaudray F, Salmon I, Braun M Y. NK cell regulation of CD4 T cell-mediated graft-versus-host disease. J Immunol. 2010; 184 (12): 6790-8.
24. Seo A N, Lee H J, Kim E J, Kim H J, Jang M H, Lee H E, et al. Tumour-infiltrating CD8+ lymphocytes as an independent predictive factor for pathological complete response to primary systemic therapy in breast cancer. Br J Cancer. 2013; 109 (10): 2705-13.
25. Hadrup S, Donia M, Thor Straten P. Effector CD4 and CD8 T cells and their role in the tumor microenvironment. Cancer microenvironment: official journal of the International Cancer Microenvironment Society. 2013; 6 (2): 123-33.
26. Kim S T, Jeong H, Woo O H, Seo J H, Kim A, Lee E S, et al. Tumor-infiltrating lymphocytes, tumor characteristics, and recurrence in patients with early breast cancer. American journal of clinical oncology. 2013; 36 (3): 224-31.
27. Robbins S H, Bessou G, Cornillon A, Zucchini N, Rupp B, Ruzsics Z, et al. Natural killer cells promote early CD8 T cell responses against cytomegalovirus. PLOS pathogens. 2007; 3 (8): e123.
28. Wodarz D, Sierro S, Klenerman P. Dynamics of killer T cell inflation in viral infections. Journal of the Royal Society, Interface. 2007; 4 (14): 533-43.
29. Bui V T, Tseng H C, Kozlowska A, Maung P O, Kaur K, Topchyan P, et al. Augmented IFN-gamma and TNF-alpha Induced by Probiotic Bacteria in NK Cells Mediate Differentiation of Stem-Like Tumors Leading to Inhibition of Tumor Growth and Reduction in Inflammatory Cytokine Release; Regulation by IL-10. Frontiers in immunology. 2015; 6:576.
30. Kaur K, Topchyan P, Kozlowska A K, Ohanian N, Chiang J, Maung P O, et al. Super-charged NK cells inhibit growth and progression of stem-like/poorly differentiated oral tumors in vivo in humanized BLT mice; effect on tumor differentiation and response to chemotherapeutic drugs. Oncoimmunology. 2018; 7 (5): e1426518.
31. Kaur K, Cook J, Park S H, Topchyan P, Kozlowska A, Ohanian N, et al. Novel Strategy to Expand Super-Charged NK Cells with Significant Potential to Lyse and Differentiate Cancer Stem Cells: Differences in NK Expansion and Function between Healthy and Cancer Patients. Front Immunol. 2017; 8:297.
32. Kabelitz D. Expression and function of Toll-like receptors in T lymphocytes. Curr Opin Immunol. 2007; 19 (1): 39-45.
33. Nouri Y, Weinkove R, Perret R. T-cell intrinsic Toll-like receptor signaling: implications for cancer immunotherapy and CAR T-cells. Journal for ImmunoTherapy of Cancer. 2021; 9 (11): e003065.
34. Reynolds J M, Pappu B P, Peng J, Martinez G J, Zhang Y, Chung Y, et al. Toll-like Receptor 2 Signaling in CD4+ T Lymphocytes Promotes T Helper 17 Responses and Regulates the Pathogenesis of Autoimmune Disease. Immunity. 2010; 32 (5): 692-702.
35. Lauzon N M, Mian F, Mackenzie R, Ashkar A A. The direct effects of Toll-like receptor ligands on human NK cell cytokine production and cytotoxicity. Cell Immunol. 2006; 241 (2): 102-12.
36. Adib-Conquy M, Scott-Algara D, Cavaillon J M, Souza-Fonseca-Guimaraes F. TLR-mediated activation of NK cells and their role in bacterial/viral immune responses in mammals. Immunol Cell Biol. 2014; 92 (3): 256-62.
37. Chalifour A, Jeannin P, Gauchat J F, Blaecke A, Malissard M, N′Guyen T, et al. Direct bacterial protein PAMP recognition by human NK cells involves TLRs and triggers alpha-defensin production. Blood. 2004; 104 (6): 1778-83.
38. Pandey S, Kawai T, Akira S. Microbial sensing by Toll-like receptors and intracellular nucleic acid sensors. Cold Spring Harb Perspect Biol. 2014; 7 (1): a016246.
39. Vabulas R M, Ahmad-Nejad P, da Costa C, Miethke T, Kirschning C J, Häcker H, et al. Endocytosed HSP60s use toll-like receptor 2 (TLR2) and TLR4 to activate the toll/interleukin-1 receptor signaling pathway in innate immune cells. J Biol Chem. 2001; 276 (33): 31332-9.
40. Ohashi K, Burkart V, Flohé S, Kolb H. Cutting edge: heat shock protein 60 is a putative endogenous ligand of the toll-like receptor-4 complex. J Immunol. 2000; 164 (2): 558-61.
41. Okamura Y, Watari M, Jerud E S, Young D W, Ishizaka S T, Rose J, et al. The extra domain A of fibronectin activates Toll-like receptor 4. J Biol Chem. 2001; 276 (13): 10229-33.
42. Termeer C, Benedix F, Sleeman J, Fieber C, Voith U, Ahrens T, et al.

Oligosaccharides of Hyaluronan activate dendritic cells via toll-like receptor 4. J Exp Med. 2002; 195 (1): 99-111.
43. Rahman A H, Taylor D K, Turka L A. The contribution of direct TLR signaling to T cell responses. Immunol Res. 2009; 45 (1): 25-36.
44. Yeap W H, Wong K L, Shimasaki N, Teo E C Y, Quek J K S, Yong H X, et al. CD16 is indispensable for antibody-dependent cellular cytotoxicity by human monocytes. Scientific Reports. 2016; 6 (1): 34310.
45. Bhatnagar N, Ahmad F, Hong H S, Eberhard J, Lu I N, Ballmaier M, et al.

FcgammaRIII (CD16)-mediated ADCC by NK cells is regulated by monocytes and FcgammaRII (CD32). Eur J Immunol. 2014; 44 (11): 3368-79.
46. Oboshi W, Watanabe T, Matsuyama Y, Kobara A, Yukimasa N, Ueno I, et al. The influence of NK cell-mediated ADCC: Structure and expression of the CD16 molecule differ among FcgammaRIIIa-V158F genotypes in healthy Japanese subjects. Hum Immunol. 2016; 77 (2): 165-71.
47. Nimmerjahn F, Ravetch J V. Fcgamma receptors as regulators of immune responses. Nat Rev Immunol. 2008; 8 (1): 34-47.
48. Kaur K, Chen P C, Ko M W, Mei A, Chovatiya N, Huerta-Yepez S, et al. The Potential Role of Cytotoxic Immune Effectors in Induction, Progression and Pathogenesis of Amyotrophic Lateral Sclerosis (ALS). Cells. 2022; 11 (21).
49. Bui V T, Tseng H C, Kozlowska A, Maung P O, Kaur K, Topchyan P, et al. Augmented IFN-γ and TNF-α Induced by Probiotic Bacteria in NK Cells Mediate Differentiation of Stem-Like Tumors Leading to Inhibition of Tumor Growth and Reduction in Inflammatory Cytokine Release; Regulation by IL-10. Front Immunol. 2015; 6:576.
50. Tseng H C, Arasteh A, Paranjpe A, Teruel A, Yang W, Behel A, et al. Increased lysis of stem cells but not their differentiated cells by natural killer cells; de-differentiation or reprogramming activates NK cells. PloS one. 2010; 5 (7): e11590.
51. Tseng H C, Bui V, Man Y G, Cacalano N, Jewett A. Induction of Split Anergy Conditions Natural Killer Cells to Promote Differentiation of Stem Cells through Cell-Cell Contact and Secreted Factors. Frontiers in immunology. 2014; 5:269.
52. Tseng H C, Inagaki A, Bui V T, Cacalano N, Kasahara N, Man Y G, et al. Differential Targeting of Stem Cells and Differentiated Glioblastomas by NK Cells. Journal of Cancer. 2015; 6 (9): 866-76.
53. Bui V T, Tseng H-C, Maung P O, Kozlowska A, Mann K, Topchyan P, et al. Augmented IFN-γ and TNF-α Induced by Probiotic Bacteria in NK Cells Mediate Differentiation of Stem-Like Tumors Leading to Inhibition of Tumor Growth and Reduction in Inflammatory Cytokine Release; Regulation by IL-10. Frontiers in immunology. 2015; 6.
54. Jewett A, Bonavida B. Target-induced inactivation and cell death by apoptosis in a subset of human NK cells. J Immunol. 1996; 156 (3): 907-15.
55. Jewett A, Wang M Y, Teruel A, Poupak Z, Bostanian Z, Park N H. Cytokine dependent inverse regulation of CD54 (ICAM1) and major histocompatibility complex class I antigens by nuclear factor kappaB in HEp2 tumor cell line: effect on the function of natural killer cells. Hum Immunol. 2003; 64 (5): 505-20.

### INCORPORATION BY REFERENCE

All publications, patents, and patent applications mentioned herein are hereby incorporated by reference in their entirety as if each individual publication, patent or patent application was specifically and individually indicated to be incorporated by reference. In case of conflict, the present application, including any definitions herein, will control.

Also incorporated by reference in their entirety are any polynucleotide and polypeptide sequences which reference an accession number correlating to an entry in a public database, such as those maintained by The Institute for Genomic Research (TIGR) on the world wide web at tigr.org and/or the National Center for Biotechnology Information (NCBI) on the World Wide Web at ncbi.nlm.nih.gov.

### EQUIVALENTS

Those skilled in the art will recognize, or be able to ascertain using no more than routine experimentation, many equivalents to the specific embodiments of the invention described herein. Such equivalents are intended to be encompassed by the following claims.

What is claimed is:

1. A composition comprising at least two bacterial strains selected from: Bifidobacterium longum, Bifidobacterium breve, and Bifidobacterium infantis, optionally further comprising at least one excipient.

2. The composition of claim 1, wherein the composition comprises Bifidobacterium longum, Bifidobacterium breve, and Bifidobacterium infantis.

3. The composition of claim 1 or 2, wherein
(a) at least about 10% but no more than about 70% of the bacteria in the composition are Bifidobacterium Longum;
(b) at least about 1% but no more than about 50% of the bacteria in the composition are Bifidobacterium breve; and/or
(c) at least about 10% but no more than about 70% of the bacteria in the composition are Bifidobacterium infantis,
wherein the percent bacteria refers to the percentage of the colony forming units (CFU) of said bacteria relative to the total CFU of bacteria in the composition.

4. The composition of any one of claims 1-3, wherein the bacteria in the composition comprise about 50% Bifidobacterium Longum, about 10% Bifidobacterium breve, and about 40% Bifidobacterium infantis, wherein the percent bacteria refers to the percentage of the CFU of said bacteria relative to the total CFU of bacteria in the composition.

5. The composition of any one of claims 1-4, wherein the composition comprises at least about 1×10⁴ but no more than about 600×10⁹ total CFU of bacteria per gram of the composition.

6. The composition of any one of claims 1-5, wherein the composition comprises at least about 180×10⁹ but no more than about 270×10⁹ total CFU of bacteria per gram of the composition, preferably wherein the composition comprises about 250×10⁹ total CFU of bacteria per gram of the composition.

7. The composition of any one of claims 1-6, further comprising at least one immunomodulatory agent.

8. The composition of claim 7, wherein the at least one immunomodulatory agent is selected from: a steroid (e.g., prednisone, methylprednisolone, dexamethasone), colchicine, hydroxychloroquine, sulfasalazine, dapsone, methotrexate, mycophenolate mofetil, azathioprine, an IL-1 inhibitor (e.g., anakinra, canakinumab, rilonacept), a TNF inhibitor (e.g., infliximab, adalimumab, golimumab, etanercept, certolizumab), an IL-6 inhibitor (e.g., tocilizumab, sarilumab), eculizumab, a CD20 inhibitor (e.g., rituximab), belimumab, cyclosporine, abatacept, an IL-17 inhibitor (e.g., secukinumab, ixekizumab, brodalumab), an IL-23 inhibitor (e.g., guselkumab), an IL-12/IL-23 inhibitor (e.g., ustekinumab), an IL-5 inhibitor (e.g., mepolizumab, reslizumab, benralizumab), an IL-4/IL-13 inhibitor (e.g., dupilumab), omalizumab, vedolizumab, N-acetylcysteine (NAC), and a JAK inhibitor (e.g., tofacitinib, upadacitinib, baricitinib).

9. The composition of any one of claims 1-8, wherein the composition increases the production or secretion of IL-10, G-CSF and/or GRO-alpha by NK cells.

10. A composition comprising at least two bacterial strains selected from: Streptococcus thermophiles, Lactobacillus acidophilus, Lactobacillus plantarum, and Lactobacillus paracasei, optionally further comprising at least one excipient.

11. The composition of claim 10, wherein the composition comprises Streptococcus thermophiles, Lactobacillus acidophilus, Lactobacillus plantarum, and Lactobacillus paracasei.

12. The composition of claim 10 or 11, wherein
(a) at least about 10% but no more than about 60% of the bacteria in the composition are Streptococcus thermophiles;
(b) at least about 1% but no more than about 50% of the bacteria in the composition are Lactobacillus acidophilus;
(c) at least about 10% but no more than about 70% of the bacteria in the composition are Lactobacillus plantarum; and/or
(d) at least about 1% but no more than 40% of the bacteria in the composition are Lactobacillus paracasei,
wherein the percent bacteria refers to the percentage of the CFU of said bacteria relative to the total CFU of bacteria in the composition.

13. The composition of any one of claims 10-12, wherein the bacteria in the composition comprise about 30% Streptococcus thermophiles, about 20% Lactobacillus acidophilus, about 40% Lactobacillus plantarum, and about 10% Lactobacillus paracasei, wherein the percent bacteria refers to the percentage of the CFU of said bacteria relative to the total CFU of bacteria in the composition.

14. The composition of any one of claims 10-13, wherein the composition comprises at least about 1×10⁴ but no more than about 600×10⁹ total CFU of bacteria per gram of the composition.

15. The composition of any one of claims 10-14, wherein the composition comprises about 250×10⁹ total CFU of bacteria per gram of the composition.

16. The composition of any one of claims 10-15, further comprising at least one cancer therapy.

17. The composition of claim 16, wherein the at least one cancer therapy is chemotherapy, radiotherapy, or immunotherapy.

18. The composition of claim 17, wherein the chemotherapy is CDDP, Paclitaxel (PTX), and/or N-acetylcysteine (NAC).

19. The composition of claim 17, wherein the immunotherapy inhibits an immune checkpoint.

20. The composition of claim 19, wherein the immune checkpoint is selected from CTLA-4, PD-1, VISTA, B7-H2, B7-H3, PD-L1, B7-H4, B7-H6, ICOS, HVEM, PD-L2, CD160, gp49B, PIR-B, KIR family receptors, TIM-1, TIM-3, TIM-4, LAG-3, GITR, 4-IBB, OX-40, BTLA, SIRPalpha (CD47), CD48, 2B4 (CD244), B7.1, B7.2, ILT-2, ILT-4, TIGIT, HHLA2, butyrophilins, and A2aR.

21. The composition of any one of claims 17, 19, and 20, wherein the immunotherapy is selected from: atezolizumab, avelumab, durvalumab, ipilimumab, nivolumab, pembrolizumab, pidilizumab, AMP-224, AMP-514, BGB-A317, STI-A1110, TSR-042, RG-7446, BMS-936559, MEDI-4736, MSB-0010718C, AUR-012, and STI-A1010.

22. The composition of any one of claims 10-21, wherein the composition increases the production or secretion by NK cells:
(a) IFN-γ, optionally wherein the composition increases the production or secretion of IFN-γ relative to the production or secretion of IL-10; and/or
(b) MCP-1.

23. The composition of any one of claims 1-22, further comprising an agent capable of activating and/or expanding NK cells, optionally wherein the agent is a cytokine or an osteoclast cell.

24. The composition of any one of claims 1-23, further comprising an NK cell or a super-charged NK cell.

25. The composition of claim 24, wherein the NK cell or the super-charged NK cell is autologous or allogeneic to the subject.

26. The composition of any one of claims 1-25, wherein the composition is in a pharmaceutical or nutraceutical composition (e.g., a dietary supplement, a tablet, a capsule).

27. The composition of any one of claims 1-26, wherein the composition increases the production or secretion of at least one cytokine by NK cells.

28. The composition of claim 27, wherein the at least one cytokine is selected from: IL-10, IFN-γ, TGF-α, IL-6, IL-1ra, IL-1B, IL-12p70, G-CSF, IL-8, GM-CSF, RANTES, Eotaxin, IP-10, IL-13, MCP-1, GRO-alpha, and IL-17.

29. The composition of any one of claims 1-28, wherein the composition is administered by intravenous, intratumoral, oral, or rectal administration.

30. A kit comprising the composition of any one of claims 1-29.

31. A method of preventing or treating an inflammatory disease or an autoimmune disease in a subject, comprising administering to the subject a composition of any one of claims 1-9 and 23-29, optionally further comprising administering to the subject at least one immunomodulatory agent.

32. The method of claim 31, wherein the the inflammatory disease or an autoimmune disease is selected from: type 1 diabetes mellitus, arthritis (e.g., rheumatoid arthritis, idiopathic arthritis, active enthesitis-related arthritis), psoriasis, psoriatic arthristis, hidradenitis suppurativa, multiple sclerosis, systemic lupus erythematosus, inflammatory bowel disease (e.g., crohn's disease, ulcerative colitis), Addison's disease, Graves' disease, Sjogren's syndrome, Hashimoto's thyroiditis, myasthenia gravis, allergy, Amyotrophic lateral sclerosis (ALS), and autoimmune vasculitis.

33. The method of claim 31 or 32, wherein the inflammatory disease is ALS.

34. The method of claim 33, further comprising administering to the subject an additional therapy that treats ALS, optionally selected from riluzole, edaravone, masitinib, retigabine, and tamoxifen.

35. The method of any one of claims 31-34, wherein the method increases the production or secretion of at least one cytokine in the subject.

36. The method of claim 35, wherein the at least one cytokine is selected from: IL-10, IFN-γ, TGF-α, IL-6, IL-1ra, IL-1B, IL-12p70, G-CSF, IL-8, GM-CSF, RANTES, Eotaxin, IP-10, IL-13, MCP-1, GRO-alpha, and IL-17.

37. The method of claim 35 or 36, wherein the at least one cytokine is selected from IL-10, G-CSF, and GRO-alpha, optionally IL-10.

38. A method of increasing the production or secretion of IL-10, G-CSF, and/or GRO-alpha in a subject, the method comprising administering to the subject a composition of any one of claims 1-9 and 23-29.

39. A method of increasing the production or secretion of IL-10, G-CSF, and/or GRO-alpha by NK cells, the method comprising contacting the NK cells with a composition of any one of claims 1-9 and 23-29.

40. The method of claim 39, wherein the method is performed in vitro, ex vivo, or in vivo.

41. A method of preventing or treating a cancer in a subject, the method comprising administering to the subject a composition of any one of claims 10-29.

42. The method of claim 41, further comprising administering conjointly to the subject at least one cancer therapy.

43. The method of claim 42, wherein the at least one cancer therapy is chemotherapy, radiotherapy, or immunotherapy.

44. The method of claim 43, wherein the chemotherapy is CDDP, Paclitaxel (PTX), and/or N-acetylcysteine (NAC).

45. The method of claim 43, wherein the immunotherapy inhibits an immune checkpoint.

46. The method of claim 45, wherein the immune checkpoint is selected from CTLA-4, PD-1, VISTA, B7-H2, B7-H3, PD-L1, B7-H4, B7-H6, ICOS, HVEM, PD-L2, CD160, gp49B, PIR-B, KIR family receptors, TIM-1, TIM-3, TIM-4, LAG-3, GITR, 4-IBB, OX-40, BTLA, SIRPalpha (CD47), CD48, 2B4 (CD244), B7.1, B7.2, ILT-2, ILT-4, TIGIT, HHLA2, butyrophilins, and A2aR.

47. The method of any one of claims 43, 45, and 46, wherein the immunotherapy is selected from: atezolizumab, avelumab, durvalumab, ipilimumab, nivolumab, pembrolizumab, pidilizumab, AMP-224, AMP-514, BGB-A317, STI-A1110, TSR-042, RG-7446, BMS-936559, MEDI-4736, MSB-0010718C, AUR-012, and STI-A1010.

48. The method of any one of claims 41-47, further comprising administering conjointly to the subject an agent capable of activating and/or expanding NK cells, optionally wherein the agent is a cytokine or an osteoclast cell.

49. The method of any one of claims 41-48, further comprising administering to the subject an NK cell or a super-charged NK cell.

50. The method of claim 49, wherein the NK cell or the super-charged NK cell is autologous or allogeneic to the subject.

51. The method of any one of claims 41-50, wherein the cancer is selected from multiple myeloma, prostate cancer, stomach cancer, bladder cancer, esophageal cancer, cervical cancer, liver cancer, kidney cancer, bone cancer, brain cancer, leukemia, head and neck cancer, oral cancer, pancreatic cancer, lung cancer, colon cancer, melanoma, breast cancer, ovarian cancer, and glioblastoma.

52. The method of any one of claims 41-51, wherein the cancer is selected from oral cancer, pancreatic cancer, lung cancer, colon cancer, melanoma, breast cancer, ovarian cancer, and glioblastoma.

53. The method of any one of claims 41-52, wherein the composition is administered at least twice to the subject.

54. The method of any one of claims 41-53, wherein the method inhibits proliferation of a cancer cell and/or increases cancer cell differentiation.

55. The method of any one of claims 41-54, wherein the method increases the production or secretion of at least one cytokine in the subject.

56. The method of claim 55, wherein the at least one cytokine is selected from: IL-10, IFN-γ, TGF-α, IL-6, IL-1ra, IL-1B, IL-12p70, G-CSF, IL-8, GM-CSF, RANTES, Eotaxin, IP-10, IL-13, MCP-1, GRO-alpha, and IL-17.

57. The method of claim 55 or 56, wherein the composition increases the production of IFN-γ, optionally wherein the composition increases the production of IFN-γ relative to the production of IL-10.

58. The method of any one of claims 31-57, wherein the subject is healthy or diseased (e.g., afflicted with an inflammatory disease, autoimmune disease, or cancer).

59. The method of any one of claims 31-58, wherein the composition is administered by intravenous, intratumoral, oral, or rectal administration.

60. The method of any one of claims 31-59, wherein the subject is a mammal, preferably wherein the mammal is a mouse or human.
